(19) United States
(12) Patent Application Publication
(10) Pub. No.: US 2023/0073514 A1
(43) Pub. Date: Mar. 9, 2023

(54) WHEAT HAPLOID INDUCER PLANT AND USES

(71) Applicant: LIMAGRAIN EUROPE, Saint-Beauzire (FR)

(72) Inventors: Jordi COMADRAN, Riom (FR); Jean-Pierre MARTINANT, Vertaizon (FR); Wyatt PAUL, Chappes (FR); Sébastien SPECEL, Montpensier (FR)

(21) Appl. No.: 17/794,052

(22) PCT Filed: Jan. 20, 2021
(86) PCT No.: PCT/EP2021/051160

§ 371 Date: Jul. 20, 2022

(30) Foreign Application Priority Data
Jan. 21, 2020 (EP) 20305047.1

(51) Int. Cl. A01H 5/10; C12N 15/82

(52) U.S. Cl. CPC A01H 5/10; C12N 15/82

(57) ABSTRACT

The invention relates to a wheat haploid inducer plant comprising at least one cell which presents inhibition of the expression of the three NLD genes of genome A, B and D, and at least one dominant or semi-dominant genetic marker, wherein said genetic marker produces, a detectable phenotype, as well as methods of uses.

The invention is in the field of plant genetics and plant breeding. The invention more specifically relates to wheat haploid inducer plant and their uses.

### BACKGROUND

The establishment of homozygous lines is a fundamental practice in plant breeding. One of the major constraints in the establishment of homozygous lines is the long time (usually 8-10 generations) needed for obtaining individuals with a high level of homozygosity by recurrent selfing.

Natural haploid inducer lines have been identified in maize allowing intraspecific crosses to produce haploids (Coe, 1959; Liu et al., 2016, Chaikam et al. 2019). Maize haploid inducer lines possess the ability to induce the development of the egg cell into a haploid embryo (containing only the haploid maternal genome) on a maize line of interest upon pollination with the inducer pollen. This process is called in vivo gynogenesis

In maize, doubled haploids represent a major breeding tool and is widely used (Geiger et al., Doubled haploids in hybrid maize breeding, Maydica, 54(4):485-499, 2009 and Röber et al., In vivo haploid induction in maize—Performance of new inducers and significance of doubled haploid lines in hybrid breeding, Maydica, 50(3-4): 275-283, 2005). It allows the rapid production of a homozygous line in fewer generations than traditional methods, can be used to benefit of a maximum genetic variance in breeding programs and to accelerate the stacking of genes of interest in a recurrent line. In maize, it has been found, by three independent studies, that the ability of the inducer lines to trigger the in vivo gynogenesis is conferred by a mutation in a pollen-specific gene encoding a predicted phospholipase A. (Gilles et al, 2017a and b), Kelliher et al., 2017; Liu et al., 2017 and US2017067067). It is named ZmNLD or also ZmMTL and ZmPLA1. Zhong et al. 2019 demonstrates that the haploid induction trait is not solely determined by the mutation of NLD. For example, in maize, a mutation in the ZmDMP, combined with the nld mutation gene can increase the haploid induction rate.

Taking advantage of the close relation between maize and rice gene structures and genome synteny, a haploid inducer line was recently obtained using a CRISPR tool in rice (Yao et al., 2018). Two constructs were designed to target respectively exon 1 and exon 4 of the OspPLAIIp gene encoding for OsMATL protein. Both generated mutants are able to produce haploids via intraspecific crosses with a haploid inducer rate of around 6%.

In wheat, breeders were not able to identify an inducer line. The wheat breeder community relies on technical processes to produce doubled haploid lines.

Several processes have been described in the literature (Tadesse et al., 2013, Hussain et al., 2012, El-Hannawy et al., 2011). They mainly describe doubled haploid technologies developed from anther or from microspores in vitro culture. Another method consists in pollinating wheat spikes with corn pollen (Niu, Z. et al. 2014). This method generally includes a step of embryo rescue. These technologies can be labor-intensive, time-consuming and species and genotype-dependent.

In Liu et al., 2019 (Extension of the in vivo haploid induction system from diploid maize to hexaploid wheat), the researchers obtained wheat plants presenting two mutated NLD genes on genomes A and D with the Cas9 enzyme. The haploid induction rate was about 2-3% in their bioRxiv publication. In a later publication in Plant Biotechnology Journal on the same events and seed lots, the haploid induction rate ranged from 5 to 15.66%. The teaching is unclear because they have no clear genotyping of the NLD mutations in the wheat haploid inducer floral organs.

In Liu et al., 2019 (Efficient induction of haploid plants in wheat by editing of TaMTL using an optimized Agrobacterium-mediated CRISPR system), the authors mention the obtention of mutations in the NLD genes on the three genomes generating an haploid induction rate of 18.9% in the TaMTL-edited T1 plants using CRISPR-SpCas9 system.

Wheat is a polyploid crop which raises challenges for conventional breeding but also for the use of molecular systems for performing gene modification.

Maize haploid inducer (HI) are widely used in the plant breeding industry in order to rapidly fix new genetic combinations. Since haploid induction is relatively inefficient, usually around 5-15% of kernels will germinate to give a haploid plant after crossing with the male haploid inducer line, methods are required to easily identify kernels that will rise to haploids. The most commonly used method in maize is for the haploid inducer line to contain a homozygous dominant marker gene that will give colored embryo scutellums and endosperm crowns after a normal double fertilization with maternal line. In progeny, kernels that have colored endosperms but non-colored embryos will produce haploid plants. The marker (R1-nj, Navajo) leads to the formation of anthocyanin giving a purple color to the embryo and endosperm. Since the maize pericarp of most lines is transparent, the embryo color can be assessed by visual inspection of the kernel. However, this method cannot be used for crosses of R1-nj haploid inducer lines to lines that are already colored with pigmented opaque pericarps or have dominant inhibitors of R1-nj (Chaikam et al. 2015). To overcome these issues Chaikam et al., (2016) introduced an additional dominant color marker in the haploid inducer line that colors roots (red root) and purple sheath and stem. A proposed alternative to anthocyanin production is the use of haploid inducer lines that have a high kernel oil content. Since oil is largely accumulated in the embryo kernel from the haploid cross that have low oil contents will give haploid plants. Identification of oil content in kernels via seed by seed NIRS (Near Infra-Red Spectroscopy) is feasible though requires automation (Melchinger et al., 2013, 2014).

These seed selection systems are difficult to transpose to a Wheat Haploid Inducer system. Wheat has an opaque pericarp which renders the use of a visible color marker in the embryo impractical without embryo rescue and alteration of the seed oil content and visualization by NIRS is more challenging than in maize due to the smaller seed and embryo size.

It is indeed important to have a method that can be applied quickly in a large scale. Intrinsic ploidy markers directly linked to ploidy status exist. Although it would be feasible to use plantlet by plantlet flow cytometry, stain and count chromosomes, measure stomatal length (Molenaar et al. 2019) or count chloroplasts in guard cells, these approaches (see Alsahlany et al (2019), Borrino and Powell (1988), Ho et al (1990)) Sari et al (1999)) are labor intensive and costly especially when large numbers of plantlets need to be screened.

Therefore, there is a need to develop alternative solutions to fasten the breeding process in wheat.

### SUMMARY

The invention thus relates to a wheat haploid inducer plant, which contains non-functional alleles of the NLD genes in its genomes, and a dominant or semi-dominant gene coding for a marker in order to quickly sort and identify haploid progeny and efficiently discriminate the haploid progeny from the diploid progeny.

In particular the invention relates to a wheat haploid inducer plant comprising at least one cell which presents inhibition of the expression of the three NLD genes of genome A, B and D, wherein the NLD genes of genome on A, B and D present at least 95% identity with SEQ ID NO: 3, 4 and 9 respectively, and at least one dominant or semi-dominant genetic marker, wherein said genetic marker produces, by itself or in complementation with another gene, a phenotype that can be detected. In particular, the plant comprises at least two different genetic markers from at least two different marker systems. The genetic marker is preferably selected from the group consisting of a dominant or semi-dominant visual genetic marker, such as a gene involved in anthocyanin biosynthesis, oil accumulation or quality, a gene modifying the morphology of the plant, in particular tiller number, leaf width, leaf hair presence/density, stomata density, ligule presence and cuticle aspect or size of the plant or of the embryo, a genetic marker producing a phenotype when combined with another genetic marker, such as components inducing hybrid necrosis and an inducible genetic marker such as a gene inducing pre-harvest sprouting in specific conditions or a toxin sensitivity gene.

In particular, the wheat haploid inducer plant comprises at least a mutation in one of the NLD genes of genome A, B and D that results in a frameshift in the coding sequence, notably in exon 4 of the NLD gene.

Inhibition of the expression of the NLD genes is preferably obtained by site directed mutagenesis, chemical mutagenesis, physical mutagenesis of the genes and/or introduction of a RNAi construct against the NLD genes in the genome of the plant. Also described is a method for identifying the wheat haploid inducer plant, comprising the steps of detecting mutations of the NLD genes in the A, B or D genomes of a wheat plant, and/or the presence of a vector inhibiting expression of the NLD genes, and the presence of a dominant or semi-dominant genetic marker, which is able to produce, by itself or in complementation with another gene, a phenotype that can be detected.

Is also described a method for obtaining the plant as disclosed, comprising
(a) Introducing, in the genome of at least one cell of a wheat plant at least one mutation in one NLD gene of one of the A, B or D genomes, and/or a genetic construct inhibiting expression of one NLD gene so as to lead to a plant having a modified genome, and presenting inhibition of the NLD genes on the A, B and D genomes, and
(b) Introducing at least one genetic marker system in the genome of said cell of the wheat plant, so that a wheat plant comprising at least one cell which presents inhibition of the expression of the three NLD genes of its genomes A, B and D and presence of a genetic marker is obtained.

Also is described the use of the wheat plant herein disclosed as pollinator parental wheat plant to induce a haploid progeny on a female parental wheat plant.

In some embodiments, the wheat haploid inducer plant further comprises in its genome one or more expression cassettes comprising at least one gene encoding for a nuclease capable of modifying the genome, in particular a CRISPR-Cas nuclease and the plant further comprises an expression cassette comprising a polynucleotide targeting one or several specific loci of the wheat genomes so as to induce a CRISPR-Cas-mediated genome modification. Such plants can be used to perform a genetic modification in the genome of a wheat plant, wherein the wheat plant is the progeny of a cross of these wheat haploid inducer plants as a pollen provider and a second plant.

These plants are useful in ex vivo methods for identifying a haploid wheat plant within a wheat plant population, comprising the step of selecting a plant in the wheat plant population which doesn't present the phenotype associated with the marker gene system, wherein the wheat plant population consists of plants obtained after cross of the wheat haploid inducer herein disclosed as a pollen provider and of another wheat plant as the female plant.

### Definitions

By “haploid inducer plant”, one wishes to refer to a plant that is able to induce the formation of haploid embryos in a maternal plant (or female plant) upon fertilization of the maternal plant by the pollen of the haploid inducer plant. In particular, at least 2% of the embryos are haploid, preferably at least 5% of the embryos, more preferably at least 7% of the embryos, most preferably at least 10% of the embryos are haploid. The resulting haploid plants only contain the genetic information of the maternal plant.

By “non-functional allele”, one intends to refer to an allele that has been rendered non-functional by a genetic mutation. Such mutation can cause a complete lack of production of the associated gene product or a product that does not function properly (such as a truncated protein). This term also encompasses absence of the gene, such as following deletion of the entire locus of the gene.

By “gene coding for a marker”, “genetic marker”, “genetic marker system” or “marker system”, it is intended to refer to a gene coding for a product that produces, by itself or when complemented with another gene, a phenotype that can be detected, for example by an analytical method. Such phenotype may preferably be a visual phenotype, that is detectable in particular by direct vision, binocular magnifying glass, microscope or through Near Infrared Spectroscopy (NIRS, which looks at the near-infrared region of the electromagnetic spectrum) or the like. The marker gene may be expressed in the seed, the embryo, the plantlet or the plant. This would depend on the pattern of expression of such marker gene.

By “dominant gene”, it is intended to refer to a gene, the effect of which masks or overrides the effect of a different variant of the same gene on the other copy of the chromosome.

By “semi-dominant gene”, it is intended to refer to a gene, the effect of which is potentiated when in presence of another expressed allele on the other chromosome. It is reminded that semi-dominance refers to the relationship between two jointly expressed alleles that have additive effects on the phenotype. In this case, it is possible to phenotypically distinguish the presence of only one expressed allele or of two expressed alleles.

### DESCRIPTION

The invention thus relates to a wheat haploid inducer plant comprising at least one cell which presents inhibition of the expression of the three NLD genes of its genomes A, B and D, wherein the NLD gene is encoding an NLD protein of genome on A, B and D. A representative allele of the NLD genes of genome A, B and D is represented by SEQ ID NO: 3, 4 and 9 respectively. In particular, SEQ ID NO: 3 represents an allele of the NLD gene of the A genome from the Chinese Spring line. SEQ ID NO: 4 represents an allele of the NLD gene of the D genome from the Chinese Spring line. SEQ ID NO: 9 represents an allele of the NLD gene of the B genome from the Cadenza line. Such inhibition results in absence of any functional NLD protein from any of the A, B, and D genome. Consequently, the invention also relates to a wheat haploid inducer plant with no functional NLD protein.

As indicated above, the invention is preferably performed in hexaploid wheat, in particular Triticum aestivum. However, the invention can also be performed in tetraploid wheat (in particular Triticum durum). In this case, such wheat haploid inducer plant comprises at least one cell which presents inhibition of the expression of the two NLD genes of its genomes A and B. All embodiments described for the hexaploid wheat can be performed for the tetraploid wheat. Consequently, and in a general manner, the invention pertains to a (tetraploid or hexaploid) wheat haploid inducer plant comprising at least one cell which presents inhibition of the expression of the all NLD genes of its genomes A, B and D, if such genome is present. The invention thus relates to a wheat haploid inducer plant comprising at least one cell which presents inhibition of the expression of the three NLD genes of genome A, B and D, wherein the NLD genes of genome on A, B and D present at least 95% identity with SEQ ID NO: 3, 4 and 9 respectively.

In the most preferred embodiment, the cell also contains at least one dominant or semi-dominant genetic marker, wherein said genetic marker induces a phenotypic trait that allows the sorting of haploids and diploids amongst the progeny from the cross between a female parent of interest and the male inducer line.

The invention also relates to a wheat cell which presents inhibition of the expression of the three NLD genes of its genomes A, B and D (or A and B in tetraploid plants), and which preferably presents as well as at least one genetic marker as disclosed above.

In a specific embodiment, said NLD genes are inhibited in multiple cells of said wheat, wherein said inhibition in multiple cells results in an inhibition in one or multiple tissues of said wheat. In this embodiment, it is possible that the NLD genes are not inhibited in other tissues of said wheat. It is preferred when the NLD genes are inhibited in the pollen of said wheat.

In a preferred embodiment, the expression of the three NLD genes is inhibited in all cells of the wheat plant.

### NLD Genes/Alleles/Genetic Variability

It is reminded that the sequence of the genes varies between different lines of wheat (genetic diversity). Consequently, SEQ ID NO: 3, 4 and 9 are representative alleles of the NLD gene. This means that the sequence of the NLD gene may be different from these sequences in different wheat lines. However, using the information provided by such sequences SEQ ID NO: 3, 4 and 9, the person skilled in the art is able to identify the NLD genes from different wheat lines, using appropriate probes. In particular, such NLD genes in different wheat lines will present at least 90% identity, more preferably at least 95% identity, more preferably at least 97% identity, more preferably at least 98% identity, more preferably at least 98.5% identity, more preferably at least 99% identity, more preferably at least 99.5% identity with one of SEQ ID NO: 3, 4 or 9.

“Percentage of sequence identity” can be determined by comparing two optimally aligned sequences over a comparison window, where the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

In order to determine identity between two nucleic sequences, one can use the blastn algorithm (Altschul et al, (1997), Nucleic Acids Res. 25:3389-3402; Altschul et al, (2005) FEBS J. 272:5101-5109, available in particular on the NCBI website (https://blast.ncbi.nlm.nih.gov/Blast.cgi)) using the following parameters:

Max target sequences: 100

Select the maximum number of aligned sequences to display

Short queries: Automatically adjust parameters for short input sequences

Expect threshold: 10

Word size: 28

Max matches in a query range: 0

Scoring Parameters

Match/Mismatch Scores: 1, −2

Gap Costs: Linear

Filters and Masking

Filter: Low complexity regions filter: on

Mask: Mask for lookup table only: on

### Inhibition of a NLD Gene

As foreseen in the present invention, a total inhibition of a gene coding for a NLD protein in a cell indicates either that:
(i) No NLD mRNA is detected in said cell after RNA isolation and reverse transcription or Northern Blot. In particular, total inhibition is obtained when no mRNA is detected after RNA isolation and reverse transcription
(ii) No functional protein is produced in said cell. No functional protein is produced in absence of NLD mRNA (see (i)). In other cases, mRNA may be present but leads to the production of a truncated protein (as an illustration when the NLD mRNA is incomplete, in particular in case of the presence of a mutation within the gene, in an intron or in an exon). Truncated proteins can be detected by isolation of the proteins, Western Blot and detection of the size of the protein with an antibody (polyclonal or monoclonal) directed against the NLD protein.

As foreseen in the present invention, a partial inhibition of a gene coding for a NLD protein in a cell indicates NLD mRNA is detected in said cell after RNA isolation and reverse transcription or Northern Blot, but at a lower level than that detected in a cell which do not bear the determinant leading to NLD inhibition. In particular, partial inhibition is obtained when a lower level of mRNA is detected after RNA isolation and reverse transcription. In particular, partial inhibition is obtained when the level of NLD mRNA is lower than 0.9 times, more preferably lower than 0.75 times, and more preferably lower than 0.66 times of the level of NLD mRNA in a cell which does not bear the determinant leading to NLD inhibition (the term “determinant” is described below). Preferably, said cell which does not bear the determinant leading to NLD inhibition is from a plant that is isogenic (but for the presence of the determinant) to the plant from which originates the cell in which partial inhibition is to be detected. Preferably, the cells are from the same plant tissue and mRNA is isolated at the same level of development. It is indeed most preferred that the level of inhibition is compared from comparable cells, only differing from the presence or absence of the determinant.

The level of NLD mRNA can be measured as an absolute level. It is nevertheless preferred that the level of NLD mRNA is measured as a relative level, compared to other control genes. In this case the method to be used to measure the level of mRNA and to detect inhibition is as follows:
(a) mRNA is isolated from tissues in which it is supposed to be inhibited and control tissues
(b) Reverse transcription and real-time quantitative PCR are performed on said mRNA using primers that amplify the NLD gene or primers that amplify control genes. These control genes are genes which are known to be usable as control in Northern Blot analysis, as their quantity level rarely varies. One can cite actin, ubiquitin 2, EF1α genes. It is preferred that at least two control genes are used, and in particular ubiquitin 2 and EF1 α.
(c) The Cp is then calculated for each amplified sample according to methods known in the art for real-time qPCR. In particular, machines used to perform real-time qPCR usually have software which can automatically calculate this value, by calculation of the second derivative maximum.
(d) One then calculates the value equal to 2exp (Cp for NLD mRNA—Cp for control gene (or mean of Cp of the control genes). This value gives a relative level of expression for NLD mRNA as compared to the level of expression of the control gene. If this value is higher than 1, this means that there is more NLD mRNA than the control gene. If this value is lower than 1, this means that there is less NLD mRNA than the control gene.
(e) One then compares the values obtained for the cells in which NLD is inhibited (i.e. where the determinant is present) and for the cells in which NLD is at the basal level (i.e. where the determinant is absent). Ratio between these two values allows the determination of the level of inhibition of the NLD gene.

In a preferred embodiment, the inhibition has been obtained by the introduction of a “determinant” in the wheat cell. As foreseen herein, a “determinant” causes the inhibition of the NLD gene, is inheritable from generation to generation and is transmissible to other plants through crosses. Determinants will be described in more details below and include mutations and transgenes (introduced foreign DNA within the genome of the cells of the plant).

As indicated above, the inhibition may be said to be “total” or “full” (i.e. there is no more production of functional NLD protein) or “partial” (i.e. there is a decrease in the production of functional NLD protein, as compared with the production in a plant that does not contain the determinant). It is also to be noted that inhibition does not preclude production of non-functional NLD protein (such as a truncated protein, in particular in case of a non-sense mutation present in the NLD gene).

It is also possible that the wheat plant presents a total inhibition of the NLD gene in some tissues, whereas there is no or only a partial inhibition in other tissues.

In an embodiment inhibition of the NLD expression is obtained by a mutation of the NLD gene through insertion of a transposable element or of a T-DNA or following physical mutagenesis. In this embodiment, expression and/or activity of NLD is inhibited by mutagenesis of the gene coding for said protein. The inhibition can be obtained in particular by site-directed mutagenesis, chemical mutagenesis or physical mutagenesis.

The mutagenesis of the gene can take place at the level of the coding sequence or of the regulatory sequences for expression, in particular of the promoter. It is, for example, possible to delete all or part of said gene and/or to insert an exogenous sequence.

By way of illustration, mention will be made of insertional mutagenesis: a large number of individuals derived from a wheat plant that is active in terms of the transposition of a transposable element are produced, and the wheat plants in which there has been an insertion in the NLD gene are selected, for example by PCR.

It is also possible to introduce one or more point mutations with physical agents (for example radiations) or chemical agents, such as EMS or sodium azide treatment of seed, site-directed DNA nucleases or gamma irradiation. The consequences of these mutations may be to shift the reading frame and/or to introduce a stop codon into the sequence and/or to modify the level of transcription and/or of translation of the gene. In this context, use may in particular be made of techniques of the “TILLING” type (Targeting Induced Local Lesions IN Genomes; McCALLUM et al., Plant Physiol., 123, 439-442, 2000). Such mutated wheat plants are then screened, in particular by PCR, using primers located in the target gene. One can also use other screening methods, such as Southern Blots or the AIMS method that is described in WO 99/27085 (this method makes it possible to screen for insertion), by using probes that are specific of the target genes, or through methods detecting point mutations or small insertions I deletions by the use of specific endonucleases (such as Cel I, Endo I, which are described in WO 2006/010646).

It is also possible to use the CRISPR/Cas (in particular CRISPR/Cas9 (WO2014093661 or WO2013176772) and CRISPR/Cas12a (WO2016205711)) system to introduce the mutation in the wheat genomes so as to inhibit expression of the NLD gene.

In this embodiment, the determinant as mentioned above is the mutation. It is indeed inheritable and transmissible by crosses. In order to allow inhibition of the genes, genes are mutated on both chromosomes of all three genomes A, B and D. It is however possible for the plant to present a mutation in the NLD genes in the two chromosomes of two wheat genomes whereas a mutation is present for a NLD gene only on one chromosome for the other genome. In particular, the mutations are homozygous for two genomes and heterozygous for the other genome. This may favor fertility of the wheat haploid inducer plant. Even though this may reduce the rate of haploid induction, it is to be noted that 50% of the pollen produced by such wheat haploid plant would be null for the three copies of the NLD gene (whereas 50% of the pollen would be null for two copies of the NLD genes with the other copy present in the gametes). It is thus expected that the rate of haploid induction would remain acceptable for industrial purposes. In one embodiment, the wheat haploid plant presents an inhibiting mutation in both copies of the NLD gene for genomes A and B and of an inhibiting mutation for only one copy of the NLD gene on the D genome. In another embodiment, the wheat haploid plant presents an inhibiting mutation in both copies of the NLD gene for genomes A and D and of an inhibiting mutation for only one copy of the NLD gene on the B genome. In one embodiment, the wheat haploid plant presents an inhibiting mutation in both copies of the NLD gene for genomes D and B and of an inhibiting mutation for only one copy of the NLD gene on the A genome.

Similarly, a preferred tetraploid wheat plant can be obtained with mutated NLD genes at a homozygous state on one of the genomes and heterozygous state on the other genome.

It is to be noted that this kind of mutations (both chromosomes of all three genomes) herein introduced are not found in nature as this leads to a non-favorable phenotype (induction of haploid progeny and reduction of the fertility) which is thus naturally not selected.

In another embodiment, inhibition of the NLD expression is due to the presence in the cell of said wheat of an antisense, or overexpression (leading to co-suppression), or RNAi construct. The DNA constructs used in these methods are introduced in the genome of said wheat plant by transgenesis, through methods known in the art. In particular, it is possible to cite methods of direct transfer of genes such as direct micro-injection into plant embryos, vacuum infiltration or electroporation, direct precipitation by means of PEG or the bombardment by gun of particles covered with the plasmid DNA of interest. It is preferred to transform the wheat with a bacterial strain, in particular Agrobacterium, in particular Agrobacterium tumefaciens.

In particular, inhibition may be obtained by transforming a wheat plant with a vector containing a sense or antisense construct. These two methods (co-suppression and antisense method) are well known in the art to permit inhibition of the target gene.

One can also use the RNA interference (RNAi) method, which is particularly efficient for the diminution of gene expression in plants (Helliwell and Waterhouse, 2003). This method is well known by the person skilled in the art and comprises transformation of the wheat plant with a construct producing, after transcription, a double-stranded duplex RNA, one of the strands of which being complementary of the mRNA of the target gene.

In this case, the determinant is the construct as described above.

In this case, one should include either a determinant that is able to inhibit expression for all three NLD genes, or a specific determinant for each NLD gene. In this case, although introduction of the determinant(s) in only one chromosome is able to inhibit expression of two chromosomal copies of the NLD genes for each genome, it is preferred when the plant is homozygous for the determinant(s), i.e. that the determinant is present on the two copies of the genome. This can be obtained by performing a self-cross of the plant regenerated after transformation with the determinant and selecting the homozygous progeny. In this case, the plant is transgenic, containing (at least) the determinant(s) as the transgene(s).

In this embodiment, the determinant as mentioned above is the DNA construct(s) (antisense, overexpression, RNAi). It is to be noted that such construct(s) is (are) not necessarily present at the same locus than the NLD genes.

It is foreseen that said nucleic acids which are in the constructs are transcribed. They are thus under the control of an appropriate promoter. One can use various promoters, among which a constitutive, or a pollen specific promoter.

In a preferred embodiment, said construct is under the control of a constitutive promoter. In a most preferred embodiment, said construct(s) is (are) RNAi construct(s), under the control of a constitutive promoter.

Other suitable promoters could be used. It should preferably be a pollen-specific promoter.

Examples of constitutive promoters useful for expression include the 35S promoter or the 19S promoter (Kay et al., 1987, Science, 236:1299-1302), the rice actin promoter (McElroy et al., 1990, Plant Cell, 2:163-171), the pCRV promoter (Depigny-This et al., 1992, Plant Molecular Biology, 20:467-479), the CsVMV promoter (Verdaguer et al., 1998, Plant Mol Biol. 6:1129-39), the ubiquitin 1 promoter of maize (Christensen et al., 1996, Transgenic. Res., 5:213) and the ubiquitin promoter from rice or sugarcane, the regulatory sequences of the T-DNA of Agrobacterium tumefaciens, including mannopine synthase, nopaline synthase, octopine synthase.

Examples of pollen-specific promoters useful for expression include the Zm13 promoter (Hamilton et al., 1992), the apg promoter from Arabidopsis thaliana (Twell et al., 1993), the Sf3 promoter (WO0055315).

It is however preferred when the determinant is a mutation in NLD genes of the A, B and D genomes that result in a frameshift (and hence of production of no NLD protein or of truncated and non-functional NLD proteins). In particular, it is preferred when the frameshift is present in exon 4 of the NLD genes.

The invention also relates to a method for obtaining a wheat haploid inducer plant, comprising the steps of
(a) Introducing at least one determinant(s) in the genome of at least one cell of a wheat plant so as to lead to a plant having a modified genome, and presenting inhibition of the NLD genes on the A, B and D genomes, and
(b) Introducing at least one genetic marker system in the genome of said cell of the wheat plant,
   so that a wheat plant comprising at least one cell which presents inhibition of the expression of the three NLD genes of its genomes A, B and D and a genetic marker is obtained.

In another embodiment, the invention also relates to a method for obtaining a wheat haploid inducer plant, comprising the steps of
(a) Introducing at least one determinant(s) in the genome of at least one cell of a wheat plant so as to lead to a plant having a modified genome, and presenting inhibition of the NLD genes on the A, B and D genomes, and
(b) Determining in vitro the presence of at least one genetic marker system in the genome of said cell of the wheat plant,
   so that a wheat plant comprising at least one cell which presents inhibition of the expression of the three NLD genes of its genomes A, B and D and a genetic marker is obtained. In this embodiment, the wheat plant in the genome of which the at least one determinant(s) is introduced already presents a genetic marker as herein described.

In another embodiment, the invention relates to a method for producing wheat haploid inducer, comprising:
(a) Introducing at least one determinant(s) inducing inhibited expression of the NLD genes on the three A, B and D wheat genomes, in the genome of at least one cell of a wheat plant;
(b) regenerating a wheat plant from the wheat cell(s) in which the determinant has been introduced; and
(c) growing the wheat plant under conditions that are suitable for expression of the determinant(s) so as to lead to a plant having a modified genome, and presenting inhibition of the NLD genes on the A, B and D genomes.

In this embodiment, at least one genetic marker system may also be introduced in the genome of said cell of the wheat plant, in step (a) or before step (b), it is also possible that, in this embodiment, the wheat plant in the genome of which the at least one determinant(s) is introduced in (a) already presents a genetic marker as herein described. The resulting plant obtained in (c) comprises at least one cell (preferably all cells) which presents inhibition of the expression of the three NLD genes of its genomes A, B and D and a genetic marker.

These methods can be performed on wheat plants in which the NLD gene is inhibited on A, B and D genomes. In this case, one or more determinant(s) (mutation or DNA construct such as RNAi construct) are introduced so as to disrupt expression of all three genes.

It can also be performed on wheat plants in which a NLD gene is already inhibited for one or two genomes. In this case, the determinant targets the NLD gene that is present on the genome in which the NLD gene is not inhibited.

In an embodiment, said determinant is a RNAi, an antisense or an overexpression construct.

In another and preferred embodiment, said determinant is a mutation introduced in the NLD genes, in particular by site-directed mutagenesis (notably by using the CRISPR/Cas system), chemical mutagenesis, or physical mutagenesis

### Markers

The wheat haploid inducer plant comprises in its genome at least one marker system. The wheat haploid inducer plant comprises in its genome one marker system or more than one marker systems.

A marker as used in the invention is a dominant or semi-dominant genetic marker present in the genome of the inducer line that allows the sorting of haploid and diploid plants, seeds, embryos, plantlets or plant tissues in the progeny of the cross between the female parent and the male inducer line.

The marker solutions proposed in order to be able to sort wheat progeny are based on post-fertilization dominant or semi-dominant selection markers that are present in the Haploid Inducer (HI) line.

Three types of marker systems are of particular interest:

### Use of a Dominant or Semi-Dominant Visual Genetic Marker

A visual marker is a marker that can be detected by looking at the plant either directly (direct vision through the human eye) or using appropriate devices (magnifier, binocular glass magnifier, microscope, NIRS, tomography). This kind of marker system is based on coloration and/or pigmentation or is a marker system affecting the morphology or the chemical composition of the embryo, seeds, plantlets or plant tissues.

Potential visual markers include dominant or semi-dominant wheat genes/loci involved in anthocyanin biosynthesis. Candidate genes include Red coleoptile (Rc), Purple culm (Pc), Purple leaf blade (Plb), Purple leaf sheath (PIs) and Red auricle (Ra) Shoeva and Khlestkina (2015). Several of these genes have now been identified and shown to be components of a transcriptional regulatory complex comprising of Myb, bHLH and WD40 proteins (Ye et al., 2017). Other visual ‘coloration’ markers include genes that effect coleoptile greening (yellow or pale coleoptile) or leaf greening; such genes include genes important for chlorophyll biosynthesis (Amato et al., 1962), for example CAOI (Chlorophyll A oxygenase 1) (Miao et al., 2013). The presence of such a dominant/semi-dominant visual marker in the haploid inducer line will lead to the visual marker being apparent only in the F1, non-haploid progeny of a cross assuming that the female lacks the visual marker. Haploid progeny can thus be distinguished from diploid progeny. Ideally the haploid inducer line will contain several different visual marker genes/loci such that it becomes less likely that the female already possesses this combination of visual markers.

Non-coloration-based visual markers based on morphological changes can be employed. For example, semi-dominant genes for plant height (eg Rht1, Rht2) are well-known (Würschum et al 2015). Strong dwarfing or elongating alleles are required such that F1 and haploid progeny can be easily distinguished. However, since the haploid inducer line will be homozygous for these strong dwarfing or elongating alleles, seed setting of the haploid inducer lines may be compromised. Other features such as tiller number, leaf width, leaf hair presence/density, stomata density, ligule presence and cuticle aspect (eg glaucocous vs glossy) might be used as visual markers.

With embryo dissection, visual or morphological markers affecting the embryo can be used to select haploid embryos. Such markers include genes for anthocyanin biosynthesis, genes affecting the shape and size of the embryo or the chemical composition of the embryo such as oil content or quality visualized for example by NIRS. An example of a marker gene that affects oil composition is FatB; mutations in FatB reduce palmitic oil content (Li et al 2011, Zheng et al 2014). In this embodiment, the haploid inducer line comprises a mutated FatB gene. The diploid progeny having the mutated FatB gene will have embryos with a reduced palmitic oil content compared to that of haploid embryos. This analysis can be performed using the NIRS technology. The selected haploid embryos can then be cultured to give haploid plantlets. Markers can also be visualized in whole seeds preventing the need for embryo dissection. Recent improvements in tomography imaging allow visualization and measurement of seed compartments (Rousseau et al (2015), Le el al (2019)), thus a screen based on dominant or semi-dominant embryo size genes present in the haploid inducer line, but not in the female parent is feasible. Similarly advances in seed by seed NIRS allow sorting of seeds based on the chemical composition of the embryo (Kandala et al 2012, Ge et al. 2020).

### Use of the Combination of Two Genetic Markers

This system consists of a binary system where one component is present in the haploid inducer male parent and the other component in the female parent. Only the F1 progeny will contain both components and thus only the F1 expresses the complete marker system and the phenotype.

An illustration of such selectable marker system with two genetic markers is the use of components that lead to hybrid necrosis. In wheat, hybrid necrosis can be caused by a combination of Ne1 and Ne2 genes in the F1 hybrid (Chu et al (2006), Zhang et al (2016)). Markers can be used to determine the Ne1 or Ne2 status of the female; if the female is Ne2 it can be crossed with an inducer line that carries a strong Ne1 allele or if the female is Ne1 it can be crossed with an inducer line that carries a strong Ne2 allele. In either case, the Ne1Ne2 progeny will not be viable and reach seed set. This method has the advantage that the selection for haploids plants and the derived doubled haploid plants is automatic, since only doubled haploid plants set seed. This system cannot be used for females that are null for both Ne1 and Ne2, however this represents a minority of female lines, most of which are Ne2 or Ne1 (Pukhal′skĩĩ et al (2010), Vikas et al (2013)).

### Inducible Marker System

Since some selection markers might be detrimental in the haploid inducer it may be preferable to employ an inducible marker system.

An example is the use of a dominant preharvest sprouting allele (eg Phs1; MKK3 n220k), see Nakamura (2018)) in the inducer line. With this allele in humid conditions seeds germinate precociously in the ear. After a cross of this haploid inducer line to a female, the seeds are left to develop in a humid environment. Germinating F1 seed can then easily be discarded enriching for seed that will develop into haploid plants.

A second example of an inducible marker system is the use of a toxin sensitivity genes in the haploid inducer line. One can cite the wheat Tsn1 gene which gives sensitivity to the peptide toxin SnToxA (Faris et al 2010, See et al 2019) or Snn1 which gives sensitivity to SnTox1 (Shi et al (2016)). The toxin can be applied to the leaves of progeny of the haploid inducer×female cross; leaf necrosis indicates that the plantlet is not a haploid providing that the female parent does not also carry the toxin sensitivity gene. Stacking Tsn1 and Snn1 in the haploid inducer line gives the option of phenotyping the progeny with either SnToxA or SnTox1 depending on which toxin sensitivity gene the female parent might carry.

As indicated above, even though it is possible to use only one marker system, it is preferred when the cells of the wheat haploid inducer plant contain genetic markers from at least two different marker systems (or marker genes). This makes it possible to avoid false positive events when the female plant also possesses one of the marker genes.

It is also preferred when the genetic marker system(s) is (are) present in a homozygous form so that each pollen cell contains a copy of such marker which is then present in the genome of all diploid progeny of the cross.

In view of the principle of induction of wheat haploid plant, the genetic marker, coming from the pollen of the wheat haploid inducer plant, will only be present in the genome of the diploid progeny (the haploid progeny contains the maternal genome). Consequently, the genetic marker is present and expressed only in diploid plants (or seeds or embryos or plantlets or tissues), and not in haploid plants (or seeds or embryos or plantlets or tissues) so that haploid progeny can be identified and sorted from diploid progeny by absence/presence of the phenotype associated with the marker.

As indicated above, preferred systems are:
(a) a marker system comprising a gene involved in anthocyanin biosynthesis, thereby coloring the diploid progeny.
(b) a marker system comprising a gene modifying the morphology of the plant, in particular tiller number, leaf width, leaf hair presence/density, stomata density, ligule presence and cuticle aspect or size of the plant (dwarfing or elongating allele) or size or shape of the embryo, so that the diploid progeny presents a specific morphological phenotype
(c) a marker system comprising a gene inducing pre-harvest sprouting in specific conditions, thereby modifying the sprouting of the diploid progeny
(d) a marker system comprising a toxin sensitivity gene, thereby rendering the diploid progeny sensitive to the toxin
(e) a marker system comprising a gene that can be complemented with another gene (or genetic sequence), so that a phenotype is expressed only when the two sequences are present in the diploid progeny.

As indicated above, the wheat haploid inducer plant is a non-naturally occurring wheat plant. This is due, in particular to the fact that inhibition of all three NLD genes is not a sustainable genotype in nature, let alone with the presence of the dominant marker gene. It is further indicated that the wheat haploid inducer plant is not exclusively obtained by means of essentially biological process. Indeed, due to the non-sustainable nature of the mutations in the NLD genes of the three genomes of the plant, (or to the presence of the genetic determinant leading to the inhibition of the genes), step of technical nature is needed to obtain these plants (introduction of the mutation by physical means, including use of nucleases in the CRISPR/Cas system or of a transgene).

In one embodiment, the marker gene or one component of a marker system is already present in the wheat plant that is intended to be modified for the inhibition of the NLD gene. In another embodiment, the marker gene or the component of a marker system is introduced by back-crossing. In a further embodiment, the marker gene of the component of a marker system is introduced by gene editing or by transformation.

In a specific embodiment, the marker is the Rc (red coleoptile) gene.

In another embodiment, the marker is the inactivated fatB gene.

In another embodiment, the marker is both the Rc and inactivated FatB genes (thereby providing two independent phenotypes to detect haploid plants in the progeny).

### Identifying the Plants Herein Disclosed

The invention also relates to a method for identifying the wheat haploid inducer plant, wherein said plant is identified by detecting the presence of the determinants in the genomes A, B or D of a wheat plant, and optionally by detecting absence of RNA of the NLD genes in cells of the wheat plant.

As indicated above, the determinant can be a transgene comprising a RNAi, overexpression of antisense sequence that leads to inhibition of the NLD genes. Such transgene can be detected by methods known in the art such as PCR or blots on the DNA of the plant, using appropriate primers or probes specifics to the transgene or the exogenous construct introduced with the plant genome.

When the determinant is a mutation, such can be detected by methods common in the art, such as sequencing of the NLD genes of the A, B and D genomes of the wheat plant. Such sequencing methods are quick, cost effective and reliable to detect mutations. Consequently, the invention also relates to a method for identifying the wheat haploid inducer plant herein disclosed, wherein said wheat plant is identified by detecting the mutation of the NLD gene of genomes A, B or D.

One can also detect the presence of the marker system by any methods available in the art to detect the presence of a given genetic sequence in the genome of a plant (PCR, sequencing, blotting the DNA . . . ). It is also possible to verify whether the genetic marker is present as a single (heterozygous) or double (homozygous) copy.

The invention also relates to a method for quality control of seed lots comprising wheat haploid inducer lines, comprising the steps of:
(a) taking a sample of seeds from a seed lot comprising wheat haploid inducer lines;
(b) conducting molecular analyses to identify and quantify the presence of haploid inducer or non-inducer alleles (and preferably including the presence of the determinant(s)), and of the marker genetic system;
(c) deducing from step b) the genetic purity value of the lot for the haploid inducer character.

Such wheat haploid inducer lines are as disclosed herein.

In a preferred embodiment, the sample of seeds at step a) comprises at least 100 seeds, at least 200, at least 300, at least 400, at least 500, at least 600, at least 700, at least 800, at least 900, at least 1000 seeds, or even more.

In a preferred embodiment, step b) is performed seed by seed or in one seed bulk or in more than one seed bulks.

Molecular analyses can be performed by using primers to amplify the NLD gene, the region comprising the mutation. Primers of the invention are the pairs SEQ ID NO: 76-77 and SEQ ID NO: 78-79. One can also use the primers of SEQ ID NO: 66-75.

### Introduction of a Nuclease

In a specific embodiment, the wheat haploid inducer plant also comprises a system for modifying the genome, in particular inducing gene editing.

The terms “gene editing” cover introduction of mutation in a gene, such as targeted mutations (mutation at a base chosen by the user) random mutation or directed mutations. One method particularly interesting for this includes inducing a DSB (Double Strand Break) and using a repair template to induce a specific nucleotide exchange during DNA repair. The CRISPR/Cas9 system is one of the specific methods of “gene editing” where the Cas9 protein and an guide RNA are used for obtaining a targeted DSB. Alternatively, a simple DSB without repair template can be made on a targeted sequence to induce random mutations at this site. These mutations should be short insertions or deletions based on NHEJ (near Homologous End Joining) or MMEJ (microhomology mediated end joining).

Consequently, in this embodiment, the wheat haploid inducer plant has further been transformed (and thus comprises in its genome, as a transgene) with one or more expression cassette(s) comprising at least one gene encoding for a nuclease capable of modifying the genome. In a preferred embodiment, the nuclease is a Cas nuclease, in particular a Cas9 nuclease, and the plant further comprises an expression cassette comprising a polynucleotide capable of targeting CRISPR-Cas genome modification. The expression cassettes are preferentially expressed in the pollen cell, using constitutive or pollen-specific promoters.

Examples of pollen-specific promoters useful for expression include the Zm13 promoter (Hamilton et al., 1992), the apg promoter from Arabidopsis thaliana (Twell et al., 1993), the Sf3 promoter (WO0055315).

In other embodiments, the nuclease is a meganuclease, a Zinc-finger nuclease or a Transcription activator-like effector nuclease (TALEN). In this embodiment, it is preferred when the nuclease is a CRISPR-Cas and when the plant further comprises an expression cassette comprising a polynucleotide targeting one or more specific loci of the wheat genome so as to induce one or more CRISPR-Cas-mediated genome modification(s).

In a further embodiment, a CRISPR-Cas enzyme unable to perform double-strand break (cut only one DNA strand or none) is coupled with a deaminase to perform base editing (Kobe WO201513355 and Harvard WO20150089406) or with a reverse transcriptase for prime editing (Anzalone et al.)

### Uses of the Wheat Haploid Inducer Plant

The invention also relates to the use of the wheat haploid inducer plant herein disclosed as pollinator parental wheat plant to induce a haploid progeny on a female parental wheat plant.

Such use can be performed by a step of harvesting pollen of the wheat haploid inducer plant and storing it until further use for pollinating a female parental plant.

The invention also relates to a process for inducing haploid wheat plant lines comprising:
(a) growing haploid inducer wheat plants, as disclosed herein;
(b) using said plants as pollinators during the crossing with a wheat female plant; and
(c) screening the progeny of the cross to select haploid plants, according to the absence of the phenotype induced by the genetic marker.

The invention also relates to a method for performing modification of a wheat plant genome comprising:
(a) Providing a wheat haploid inducer plant as herein disclosed and further comprising a nuclease as described above,
(b) Crossing the first plant with a second plant, and
(c) Recovering and selecting a haploid progeny from step (b) wherein said progeny comprises genome modification.

Without being bound by this theory, it is believed that genome modification is obtained simultaneously during the haploid induction, as a result of introduction of the nuclease and polynucleotide capable of targeting CRISPR-Cas genome modification in the cytoplasm of the ovule during the pseudo-fertilization.

This haploid progeny can undergo a chromosome doubling step resulting in the obtention of a diploid plant having the desired modification at an homozygous status. The chromosome doubling step can be performed according to the following publications (Sood et al; 2003, Niu et al; 2014, Vanous et al. 2017, Häntzschel et al. 2010, Melchinger et al. 2016, Ren 2018, Chaikam et al. 2020).

The invention also relates to the use of the wheat haploid inducer plant as herein disclosed (also comprising the nuclease in its genome) to perform a genetic modification in the genome of a wheat plant, wherein the wheat plant is obtained by providing pollen of the wheat haploid inducer plant to a second plant, and recovering and selecting an haploid progeny of the cross thereby obtain, wherein said progeny comprises a genome modification. This haploid progeny can undergo a chromosome doubling step.

The invention also relates to the use of the wheat haploid inducer plant as herein disclosed (also comprising the nuclease in its genome) to perform a genetic modification in the genome of a wheat plant, wherein the wheat plant is the progeny of a cross of the wheat haploid inducer plant also comprising a nuclease, as disclosed above, as a pollen provider and a second plant. This genetic modification is observed in the haploid plant. This haploid plant can undergo a chromosome doubling step.

The invention also encompasses a method for sorting (i.e. selecting or identifying) a haploid wheat plant within a wheat plant population, comprising the step of selecting a plant in the wheat plant population which doesn't present the phenotype associated with the marker gene system, wherein the wheat plant population consists of plants obtained after cross of the wheat haploid inducer herein disclosed as a pollen provider and of another wheat plant as the female plant. Such selection is generally visually performed. This method is an in vitro or ex vivo

### BRIEF DESCRIPTION OF THE FIGURES

FIG. 1 shows the alignment of maize Not Like Dad (NLD) protein (ZmNLD_GRMZM2G471240, SEQ ID NO: 2) with Wheat putative orthologs. TaNLD-like 4AS (SEQ ID NO: 6), TaNLD-like 4DL (SEQ ID NO: 7), TaNLD-like_4BL 3′ (SEQ ID NO: 8), TaNLD-like_BL_Cadenza (SEQ ID NO: 10) with the consensus sequence (SEQ ID NO: 83)

FIG. 2 shows the expression pattern of Wheat NLD-like genes. RNAseq data was obtained from the IWGSC. Most expression is seen in spikes at the Zadock 65 stage. All 3 NLD orthologs appear to be expressed.

FIG. 3 shows the C-terminal protein alignments of ZmNLD and TaNLDs with the haploid inducer mutated protein ZmNLD-PK6 from maize inducer line PK6. The sequences are parts of the following sequences from the sequence listing: TaNLD-like 4AS (SEQ ID NO: 6), TaNLD-like 4DL (SEQ ID NO: 7), TaNLD-like_BL_Cadenza (SEQ ID NO: 10), ZmNLD_GRMZM2G471240 (SEQ ID NO: 2), ZmNLD-PK6 (SEQ ID NO: 33)

FIG. 4 shows the position of the target sequence of the designed LbCpf1 RNA guides. The crRNA PAM TTTA lies on the reverse strand 13 bp downstream of the A residue (in bold) which is the equivalent position in the ZmNLD-PK6 gene where the frameshift occurs. A 23 bp sequence is used as a target. TaNLD_4AS_Fielder_exon4 (SEQ ID NO: 84), TaNLD_4BL_Fielder_exon4 (SEQ ID NO: 85), TaNLD_4DL_Fielder_exon4 (SEQ ID NO: 86), Consensus (SEQ ID NO: 87), target TTTN_AS+DL (SEQ ID NO: 88), target TTTN_BL (SEQ ID NO: 89).

FIG. 5 shows the construct pBIOS11170 T-DNA region for editing of the NLD gene with LbCpf1

FIG. 6 shows the summary of the number of mutations found in T0 plantlets

FIG. 7 shows the alignment of wildtype and mutant TaNLD nucleotide sequences around the targeted region in exon4. The sequences are parts of the following sequences from the sequence listing: TaNLD_4AS_Fielder_exon4 (SEQ ID NO: 11), TaNLD_4AS_Fielder_exon4_del8bp (SEQ ID NO: 21), TaNLD_4BL_Fielder_exon4 (SEQ ID NO: 13), TaNLD_4BL_Fielder_exon4_del11 bp (SEQ ID NO: 23), TaNLD_4BL_Fielder_exon4_del26 bp (SEQ ID NO: 25), TaNLD_4DL_Fielder_exon4 (SEQ ID NO: 12), TaNLD_4DL_exon4_del7 bp (SEQ ID NO: 27), TaNLD_4DL_exon4_del8bp (SEQ ID NO: 29), TaNLD_4DL_exon4_del20 bp (SEQ ID NO: 31).

FIG. 8 shows the alignment of wildtype and mutant TaNLD exon 4 protein sequences. The sequences are parts of the following sequences from the sequence listing: TaNLD_4AS_exon4 (SEQ ID NO: 14), TaNLD_4AS_exon4_del8bp (SEQ ID NO: 22), TaNLD_4BL_exon4 (SEQ ID NO: 16), TaNLD_4BL_exon4_del11 bp (SEQ ID NO: 24), TaNLD_4BL_exon4_del26 bp (SEQ ID NO: 26), TaNLD_4DL_exon4 (SEQ ID NO: 15), TaNLD_4DL_exon4_del7 bp (SEQ ID NO: 28), TaNLD_4DL_exon4_del8bp (SEQ ID NO: 30), TaNLD_4DL_exon4_del20 bp (SEQ ID NO: 32)

FIG. 9 shows the alignment of wildtype and mutant Genome D TaNLD nucleotide sequences around the targeted region in exon4. The sequences are parts of the following sequences from the sequence listing: TaNLD_4DL_Fielder_exon4 (SEQ ID NO: 12), TaNLD_4DL_Fielder_N1del_exon4 (SEQ ID NO: 34), TaNLD_4DL_Fielder_N2del_exon4 (SEQ ID NO: 35), TaNLD_4DL_Fielder_N4del_exon4 (SEQ ID NO: 36), TaNLD_4DL_Fielder_N5del_exon4 (SEQ ID NO: 37), TaNLD_4DL_Fielder_N6del_exon4 (SEQ ID NO: 38), TaNLD_4DL_Fielder_N7del_exon4 (SEQ ID NO: 39), TaNLD_4DL_Fielder_N9*del_exon4 (SEQ ID NO: 40)

FIG. 10 shows the alignment of wildtype and mutant genome D TaNLD exon 4 protein sequences. The sequences are parts of the following sequences from the sequence listing: TaNLD_4DL_exon4 (SEQ ID NO: 15), TaNLD_4DL_Fielder_N1del_exon4 (SEQ ID NO: 41), TaNLD_4DL_Fielder_N2del_exon4 (SEQ ID NO: 42), TaNLD_4DL_Fielder_N4del_exon4 (SEQ ID NO: 43), TaNLD_4DL_Fielder_N5del_exon4 (SEQ ID NO: 44), TaNLD_4DL_Fielder_N6del_exon4 (SEQ ID NO: 45), TaNLD_4DL_Fielder_N7del_exon4 (SEQ ID NO: 46), TaNLD_4DL_Fielder_N9*del_exon4 (SEQ ID NO: 47)

FIG. 11 shows the construct pBIOS11489 T-DNA region for editing of the NLD gene with SpCas9

FIG. 12 shows the alignment of wildtype and mutant TaNLD exon 4 sequences from Cas9-derived plant B0183691. The sequences are parts of the following sequences from the sequence listing: TaNLD_4AS_exon4 (SEQ ID NO: 14), TaNLD_4AS_Fielder_exon4_+1_B0183691 (SEQ ID NO: 58), TaNLD_4BL_exon4 (SEQ ID NO: 16), TaNLD_4BL_Fielder_exon4_+1_B0183691 (SEQ ID NO: 59), TaNLD_4DL_exon4 (SEQ ID NO: 15), TaNLD_4DL_Fielder_exon4_+1_B0183691 (SEQ ID NO: 60).

FIG. 13 shows alignment of wildtype and mutant TaNLD exon 4 sequences from Cas9-derived plant B0183700. The sequences are parts of the following sequences from the sequence listing: TaNLD_4AS_exon4 (SEQ ID NO: 14), TaNLD_4AS_Fielder_exon4_del1_B0183700 (SEQ ID NO: 62), TaNLD_4AS_Fielder_exon4_del4_B0183700 (SEQ ID NO: 63), TaNLD_4BL_exon4 (SEQ ID NO: 16), TaNLD_4BL_Fielder_exon4_del4_B0183700 (SEQ ID NO: 64), TaNLD_4DL_exon4 (SEQ ID NO: 15), TaNLD_4DL_Fielder_exon4_CtoA_B0183700 (SEQ ID NO: 61), TaNLD_4DL_Fielder_exon4_del4_B0183700 (SEQ ID NO: 65)

An embodiment of the invention will be described in detail in the following examples. All genes, constructs, plants described in these examples are part of the invention.

### EXAMPLES

### Example 1: Identification of Wheat Orthologs of Maize not Like Dad (NDL)

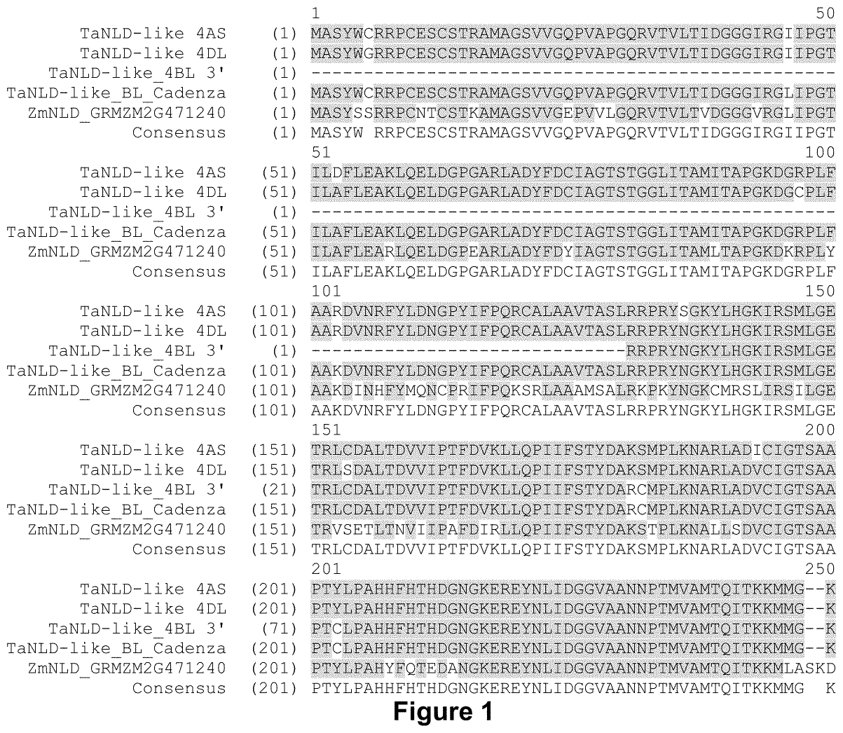

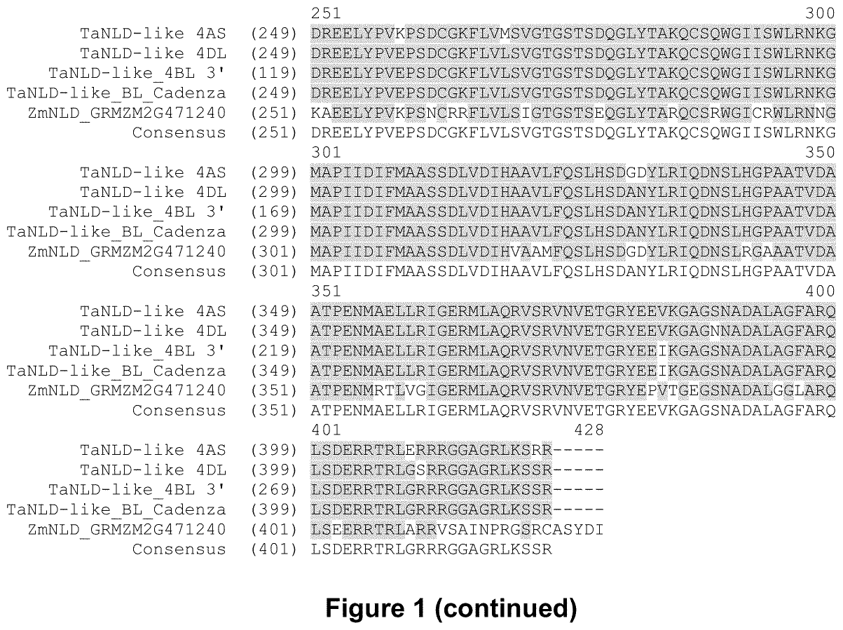

Putative orthologs of the maize B73 NLD gene (GRMZM2G471240) SEQ ID NO: 1 (WO_2016_177887, Gilles et al. (2017)) were identified by TBLASTN analysis of the Chinese Spring wheat genome sequence using the maize line B73 ZmNLD protein (SEQ ID NO: 2) as the query sequence. The best matching sequences were on chromosomes 4AS, 4DL and 4BL (SEQ ID NO: 3, SEQ ID NO: 4 and SEQ ID NO: 5). The predicted protein sequences are respectively SEQ ID NO: 6, SEQ ID NO: 7 and SEQ ID NO: 8. The 4BL genomic sequence is incomplete lacking the 5′ region of the coding sequence such that it starts in exon 2. A complete genomic sequence for the 4BL homolog was identified from a genomic sequence of the variety Cadenza (SEQ ID NO: 9) and the predicted protein sequence is SEQ ID NO: 10. An alignment of the predicted wheat sequences with ZmNLD is shown in FIG. 1. TaNLD-like 4AS has 75.2% identity with ZmNLD, TaNLD-like_4DL, 74.8% identity and TaNLD-like_4BL Cadenza, 74.8% identity.

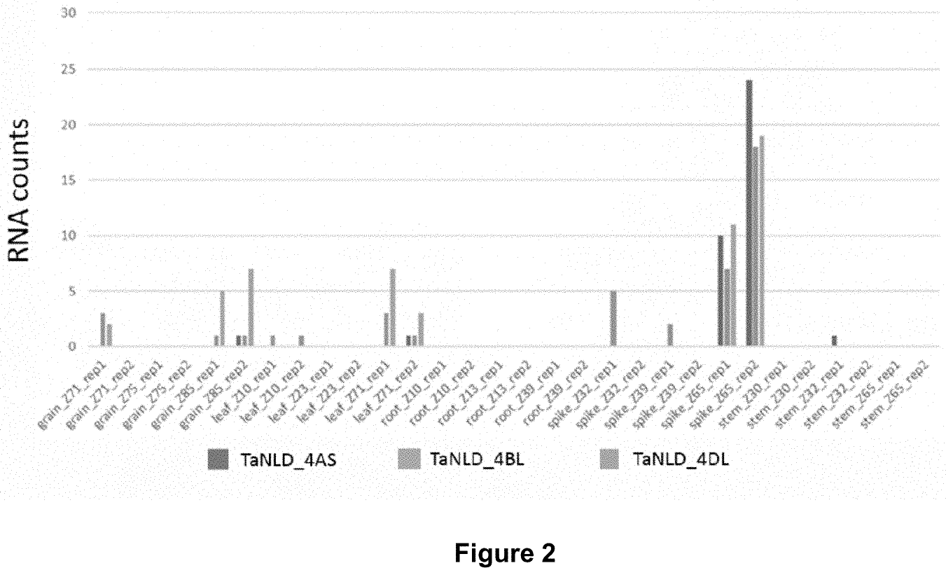

The ZmNLD gene is known to be expressed specifically in reproductive tissues; in the pollen from the bicellular stage with expression continuing in the pollen tube. It is expected that true wheat orthologs of NLD would have a similar pattern of expression. Wheat RNAseq data showed that indeed all three potential orthologs were expressed almost exclusively in reproductive tissues (late developing spike) (FIG. 2).

RNAseq data was obtained from the IWGSC (International Wheat Genome Sequencing Consortium). Most expression is seen in spikes at the Zadock 65 stage. All 3 NLD orthologs appear to be expressed.

### Example 2: Creation of Maize NLD-PK6-Like Mutations in Wheat NLD-Like Genes of the Variety Fielder Using CRISPR

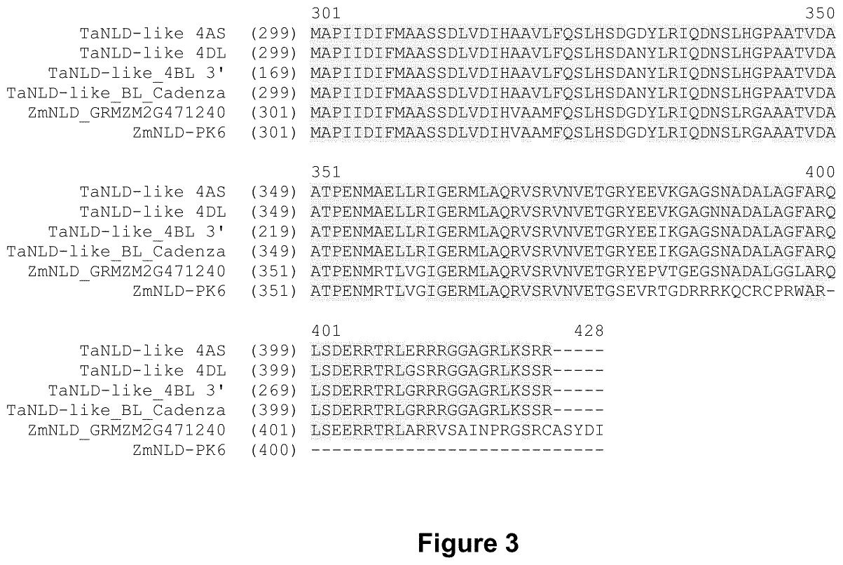

From the expression data in FIG. 2 it appears that all of the 3 identified genome copies of TaNLD are expressed. Thus, in order to phenocopy the maize NLD haploid inducer phenotype it may be necessary to mutate all 3 genes. In this example the objective is to create wheat mutations that are very similar to the NLD maize mutation which is the result of a frameshift at the 3′ end of the gene (WO_2016_177887, Gilles et al. (2017)). The site of the frameshift appears to remove a C-terminal part in the truncated NLD-PK6 protein, from maize haploid inducer line PK6, such that the protein is no longer attached to the plasma membrane (Gilles et al. (2017)). The frameshift is after the G 379 residue of the ZmNLD protein. This sequence is conserved in the TaNLD sequences and lies in exon 4 (FIG. 3). In order to design guide RNA sequences in the Fielder wheat variety to be used, exon 4 was amplified and sequenced from each of the three TaNLD genomic copies in Fielder using primers designed to the Chinese Spring sequences (Table 1). Primer pair A010430+A010435 (SEQ ID NO: 66-67) amplified the Fielder 4AS NLD gene and the primers pairs A010433 and A010423 (SEQ ID NO: 68-69) amplified both the Fielder 4BL and 4DL genes. The sequencing of cloned amplicons allowed the identification of the 4BL and 4DL gene sequences. Genome-specific NLD primers are shown in Table 1 (SEQ ID NO: 70-75). The exon 4 TaNLD-4AS, 4DL and 4BL Fielder sequences are shown respectively in SEQ ID NO:11, SEQ ID NO: 12 and SEQ ID NO: 13.

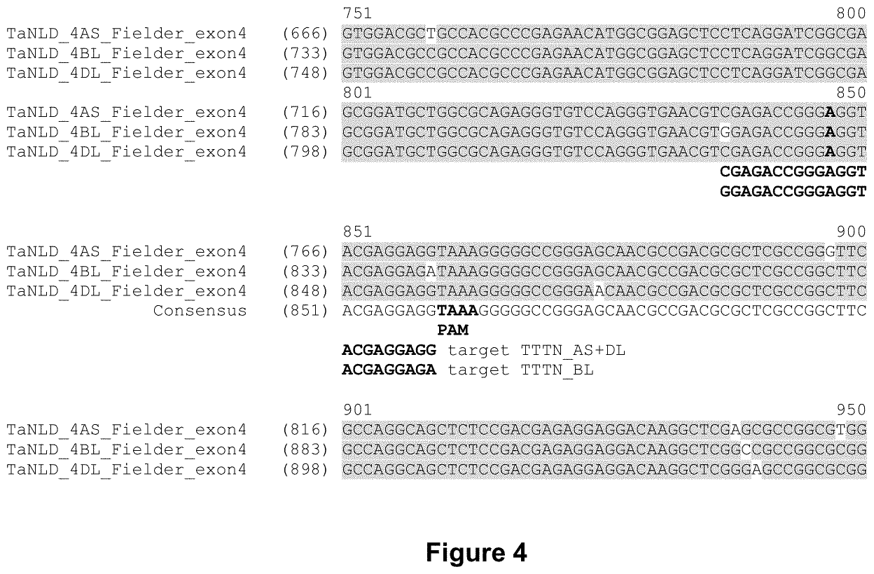

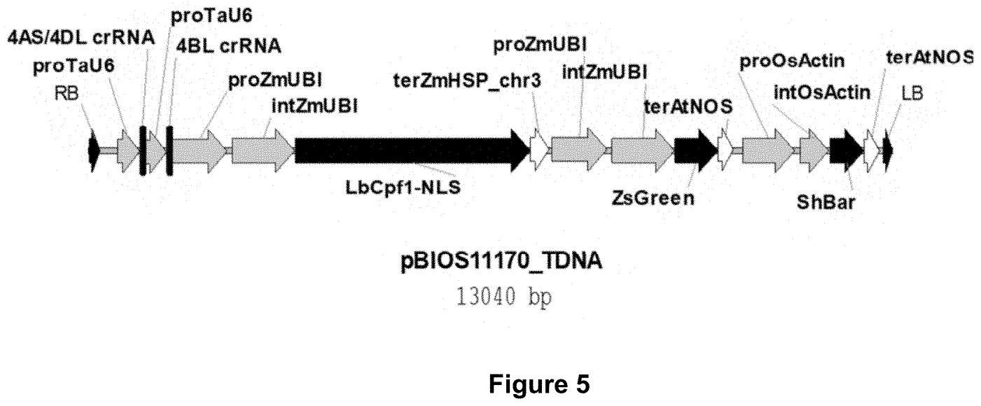

The CRISPR system Cpf1 was used to introduce mutations into the TaNLD genes. A conserved PAM sequence (TTTA) was found on the reverse strand of the TaNLD sequences 13 bp downstream of the A residue which is the equivalent position in the ZmNLD-PK6 gene where the frameshift occurs (FIG. 4). Two 23 bp sequences were used as a target, one sequence is identical to the NLD 4AS and 4DL genes (SEQ ID NO: 80 5′ CCTCCTCGTACCTCCCGGTCTCG 3′) and the other identical to the 4BL sequence (SEQ ID NO: 81 5′ TCTCCTCGTACCTCCCGGTCTCC 3′). The two sequences differ by 2 bp. A binary plant transformation construct (pBIOS11170 FIG. 5) was made that contained a Lachnospiraceae bacterium ND2006 Cpf1 gene with a C-terminal NLS and HA epitope TAG (Zetsche et al., 2015) encoding the protein SEQ ID NO: 17, expressed from the constitutive maize Ubiquitin promoter plus 5′UTR (SEQ ID NO: 18). The construct also contained a wheat U6 promoter (SEQ ID NO: 19) driving the expression of a crRNA containing the TaNLD-4AS or TaNLD-4DL target sequence (SEQ ID NO: 80) and a wheat U6 promoter (SEQ ID NO: 19) driving the expression of a crRNA containing the TaNLD-4BL target sequence (SEQ ID NO: 81). In addition, the construct contained a selectable marker gene (BAR) for plant transformation and a visual marker gene (ZsGreen) to aid the detection of transgenic events. FIG. 5 shows a schematic diagram of the T-DNA region of pBIOS11170 (SEQ ID NO: 20).

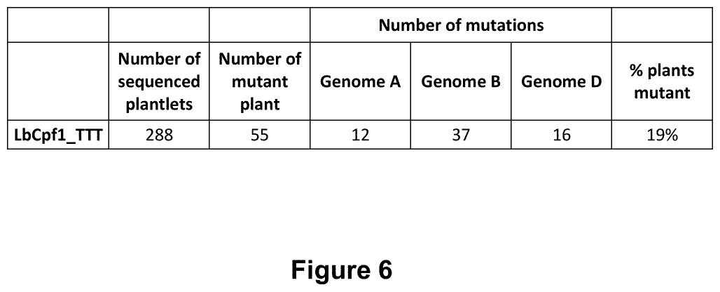

pBIOS11170 was transferred to the agrobacterial strain EHA105 giving the strain T10932 and transformed into Fielder using a protocol based on immature embryo transformation (Ishida et al.; 2015). The DNA sequence of the region targeted in Exon4 in transformed plantlets was amplified using primers that amplified all 3 NLD genome copies (Table 1, SEQ ID NO: 76-77). The amplicons obtained were sequenced using Next Generation Sequencing (NGS) and the sequences assigned to genomes based on NLD genome-specific SNPs in the amplicon. In primary transformants mutations were observed at the targeted sites in all targeted genes (FIG. 6) but no plant contained in-frame deletions in all three targeted genes. Selected plants with mutations were analyzed in the T1 generation.

T0 plant B0142293 contained a heterozygous 8bp deletion mutation in TaNLD_4AS after the position 425 bp in SEQ ID NO: 21. The predicted Exon4 protein sequence of this mutation is shown in SEQ ID NO: 22. Plant B0142293 also contained 2 different mutant 4BL sequences, a deletion of 11 bp after the position 421 bp in SEQ ID NO: 23, giving the predicted exon 4 protein sequence in SEQ ID NO: 24 and a deletion of 26 bp after the position 409 bp in SEQ ID NO: 25, giving the predicted exon 4 protein sequence in SEQ ID26. In the T1 generation plants were identified that were homozygous for the 4AS_8bp deletion (genotype represented as a8a8BBDD), homozygote for each of the 4BL mutations (AAb11b11DD and AAb26b26DD) and homozygote for the two double genome mutations (a8a8b11b11DD and a8a8b26b26DD). The mutations were inherited from the T0 in a mendelian fashion without any apparent segregation distortion.

T0 plant B0148740 contained a heterozygous 7 bp deletion mutation in TaNLD_4DL after the position 423 bp in SEQ ID NO: 27. The predicted Exon4 protein sequence of this mutation is shown in SEQ ID NO: 28.

T0 plant B0148773 contained a heterozygous 8bp deletion mutation in TaNLD_4DL after the position 423 bp in SEQ ID NO: 29. The predicted Exon4 protein sequence of this mutation is shown in SEQ ID NO: 30.

T0 plant B0164336 contained a homozygous 20 bp deletion mutation in TaNLD_4DL after the position 423 bp in SEQ ID NO: 31. The predicted Exon4 protein sequence of this mutation is shown in SEQ ID NO: 32.

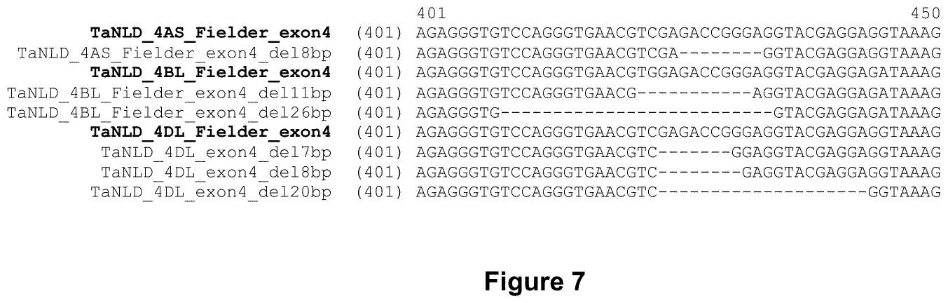

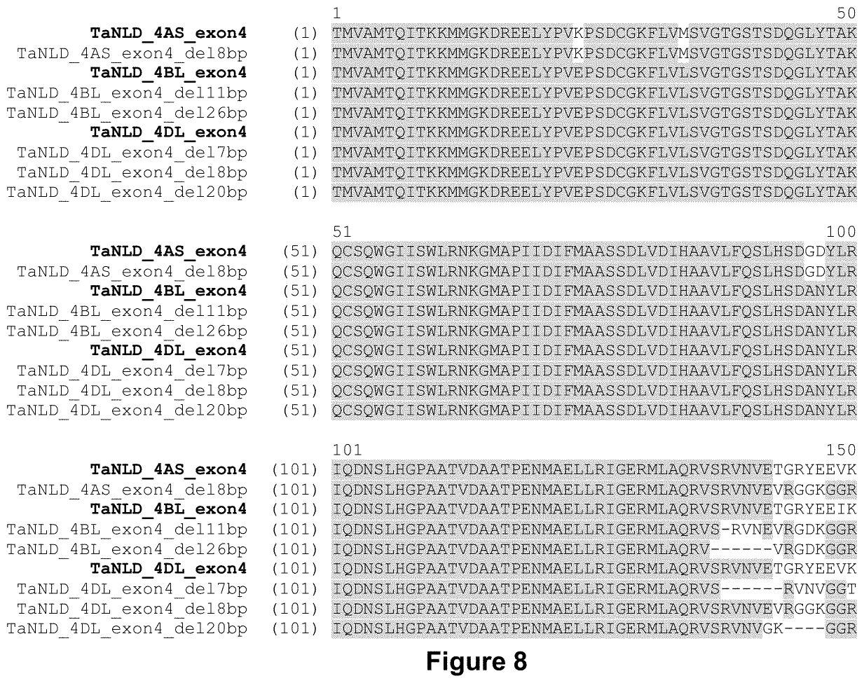

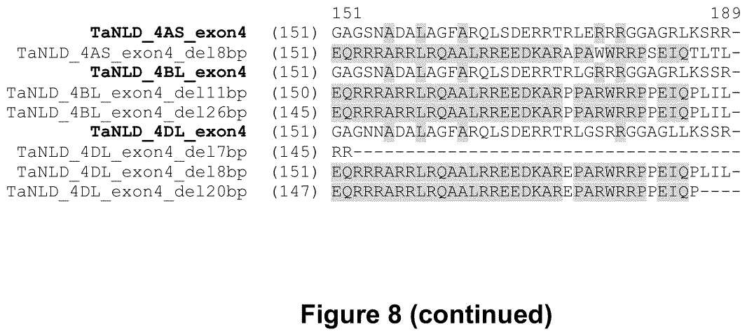

Alignments of wildtype and mutant nucleotide sequences from exon4 are shown in FIG. 7 and those of protein sequences in FIG. 8. It can be seen that the altered protein C-terminal sequences are highly different to the wild-type sequences. All but one of the mutant proteins have a similar length to the wildtype sequences and the new C-terminal sequences have significant homology. Mutant TaNDL_4DL_del7 bp is very different in that its new C-terminal protein sequence is very short (5 amino acids compared to 46 in the TaNDL_4DL protein.

To generate all possible mutant combinations the two aabbDD lines were crossed to the three AABBdd lines. The F1 plants were selfed and the F2 plants genotyped by TaNLD genome specific PCR and sequencing. Plants that are homozygous for single, double and triple TaNLD genome mutations were retained that also can lack the Cpf1 transgene construct.

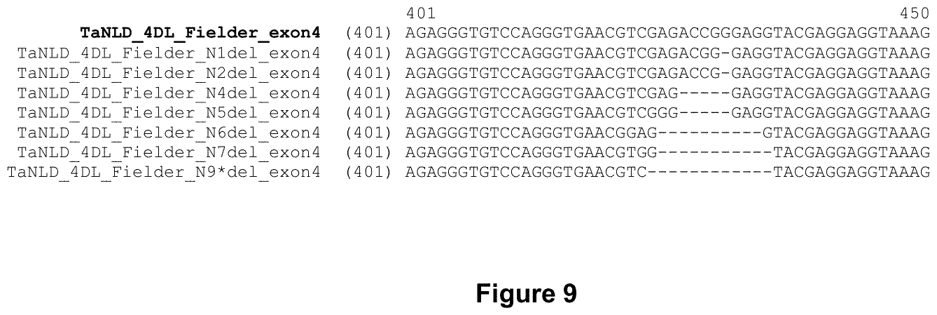

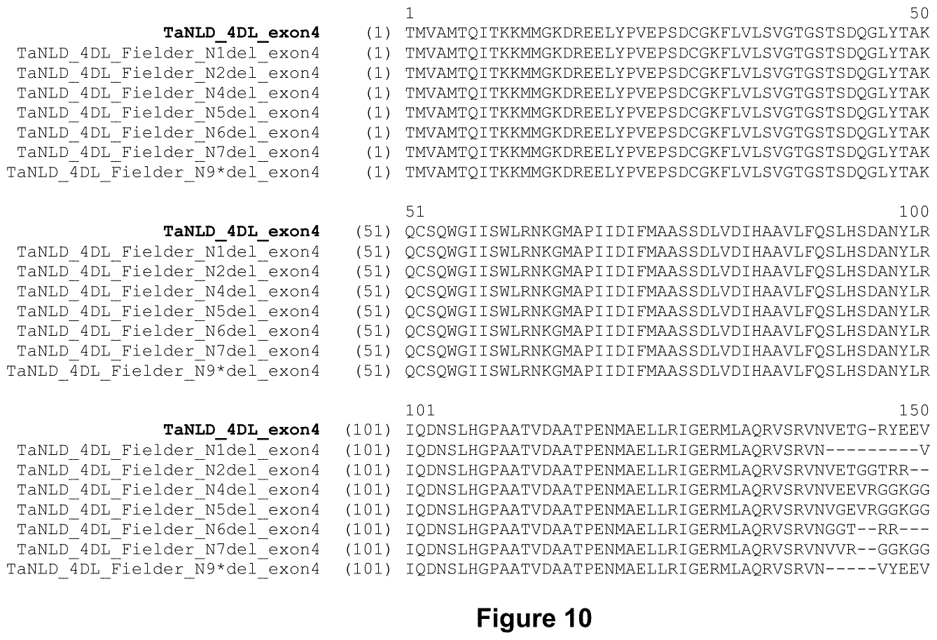

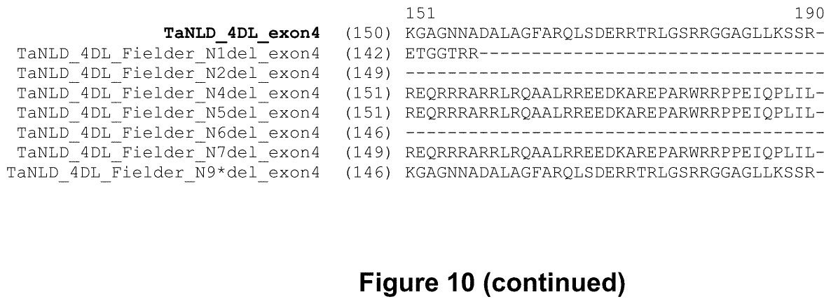

In addition to the above aabbDD×AABBdd crossing strategy aabbDD plants containing the Cpf1 transgene construct were screened for the appearance of D genome NLD mutations. 7 different D mutations were obtained (FIG. 9, DNA sequences SEQ ID NO: 34-40, and FIG. 10; predicted amino-acid sequences SEQ ID NO: 41-47), the plants selfed and triple homozygote mutant aabbdd lines identified.

TABLE 1
List of primers

| | | FORWARD PRIMER | | | REVERSE PRIMER | | |
|---|---|---|---|---|---|---|---|
| NAME | DESCRIPTION | SEQUENCES 5′-3′ | NAME | DESCRIPTION | SEQUENCES 5′-3′ | AMPLIFIES | SIZE |
| A_010430 | FOR_4AS | GACTTCACTTACGCTTCGTCAT | A_010435 | REV_EXON_4AS | GCTTGCCGAAATAGGTA | Fielder | 2235 bp |
| | | GAGCG | | | GGAGG | NLD 4AS | |
| A_010433 | FOR_4BL | GAATTAAGATCTGCCTCCTAC | A_010423 | REV_EXON4 | GAAGCTTTCTCTACCTA | Fielder | 2052 bp |
| | | CACAGTCG | | | TCCCAG | NLD 4BL | |
| | | | | | | and 4DL | |
| PP_02247_ | FOR_4AS | cttctccacatacgacgtatatatgc | PP_02247_ | RREV_4AS | atgttcccagtgttctgttgtataggt | Fielder | 1300 bp |
| F | | | R | | | NLD_4AS | |
| PP_02248_ | FOR_4BL | cgacgtatgctaattttatacgagg | PP_02248_ | REV_4BL | gctagccaaagtagggatgctg | Fielder | 1211 bp |
| F | | | R | | | NLD_4BL | |
| PP_02249_ | FOR_4DL | cgacgtatgccaattttatatgtataag | PP_02249_ | REV_4DL | gatgatcgtttaaccgatgttgg | Fielder | 1309 bp |
| F | | | R | | | NLD_4DL | |
| PP_02255_ | NGS Forw | ATCCAGGACAACTCGCTCC | PP_02255_ | NGS Rev crRNA | AGCCTTGTCCTCCTCTC | Fielder | 221 bp |
| F1 | cRNA | | R1 | | GTC | NLD 4AS/ | |
| | | | | | | 4BL/4DL | |
| PP_03021_ | NGS Forw | gactgcggcaagttcctg | PP_03021_ | NGS Rev gRNA7 | caccctggacaccctctg | Fielder | 418 bp |
| F | gRNA7 | | R | | | NLD 4 AS/ | |
| | | | | | | 4BL/4DL | |

### Example 3: Phenotype of TaNLD-PK6-Like Mutants

Double homozygote aabbDD and single homozygote mutant AABBdd plants when selfed had a normal seed set, however aabbdD and aabbdd mutants had a noticeable reduction seed set as measured by a fertility index (Table 2 and Table 3). The aabbdd mutants had a lower fertility index than the aabbdD mutants. It is known that the maize inducer lines have a reduction of set on selfing, this is thought to be due to endosperm genome imbalances where in some fertilization products, the endosperm lacks a paternal genome (Lin, (1984)). Such a 2n:0p endosperm has arrested development leading to kernel abortion. It is thus anticipated that wheat haploid inducer NLD mutant lines could also have reduced seed set. If so the aabbdd triple mutants but not the double aabbDD and single AABBdd mutants are likely to be inducers of haploidy. In addition, the selfed progeny of the aabbDd lines contained a significant proportion of plants in the triple homozygote progeny that were completely sterile (Table 3). This sterility might be due to the production of haploid plants which would be sterile. It is noticeable that the progeny of the aabbd8dN9* line had a higher level of sterility that the other lines. The d8 mutation is a frame shift whereas the dN9* mutation is an in-frame mutation of 4 amino acids. The low fertility index of the aabbdN9*dN9* progeny suggests that the dN9* mutation has an NLD loss of function. Thus, the aabbd8dN9* parental plant is a triple NLD homozygote mutant.

TABLE 2
Fertility of the aabbDd parental lines. The Fertility
Index is the number of seeds per spikelet.

| | | | | Fertility |
|---|---|---|---|---|
| Genotype parent | | Seed | Spikelets | Index |
| AABB | DD | 329 | 167 | 2 |
| aabb | DD | 620 | 347 | 1.8 |
| aabb | dN1D | 209 | 274 | 0.8 |
| aabb | dN2D | 247 | 208 | 1.2 |
| aabb | dN4D | 90 | 64 | 1.4 |
| aabb | dN5D | 115 | 88 | 1.3 |
| aabb | dN6D | 111 | 71 | 1.6 |
| aabb | dN7D | 76 | 48 | 1.6 |
| aabb | d8dN9* | 61 | 123 | 0.5 |

TABLE 3
Fertility of the aabbDd and aabbdd progeny plants.

| | | | | | | fertility |
|---|---|---|---|---|---|---|
| Genotype progeny | | Sterile | fertile | Total | % sterile | index |
| aabb | DD | 0 | 8 | 8 | 0% | 1,9 |
| aabb | dN1D | 0 | 5 | 5 | 0% | 1,1 |
| aabb | dN2D | 0 | 5 | 5 | 0% | 1,1 |
| aabb | dN4D | 0 | 10 | 10 | 0% | 1,5 |
| aabb | dN6D | 0 | 5 | 5 | 0% | 0,9 |
| aabb | dN7D | 0 | 5 | 5 | 0% | 1,1 |
| aabb | dN1dN1 | 1 | 15 | 16 | 6% | 0,4 |
| aabb | dN2dN2 | 4 | 10 | 14 | 29% | 0,5 |
| aabb | dN4dN4 | 0 | 3 | 3 | 0% | 0,9 |
| aabb | dN5dN5 | 6 | 24 | 30 | 20% | 0,7 |
| aabb | dN6dN6 | 3 | 8 | 11 | 27% | 0,7 |
| aabb | dN7dN7 | 0 | 3 | 3 | 0% | 0,8 |
| aabb | d8d8 | 5 | 7 | 12 | 42% | 0,6 |
| aabb | dN9*dN9* | 5 | 4 | 9 | 56% | 0,6 |

The fertility index is calculated according to the formula:

fertility index=(number of kernels/number of spikelets) per plant

### Example 4: Haploid Induction of TaNLD-PK6-Like Mutants

In order to determine if aabbDD lines are haploid inducers, pollen from the aab11b11DD and aab26b26DD lines were used to pollinate a Cytoplasmic Male Sterility (CMS) line. The CMS line used was seed from a cross of CMS line Arturnick to a fertile-non restorer spring cultivar. 114 plantlets from this CMS×NLD aabbDD cross (45 from aab11b11DD and 69 from the aab26b26DD cross) were genotyped for the NLD genome A and genome B mutations. All the plantlets were heterozygous for the NLD locus (mutant and WT alleles). Thus, the aabbDD lines used did not induce haploid production to a significant extent.

Pollen from the triple mutant lines was also used to pollinate a CMS line. In this case the CMS used was a BC1 between the CMS line Arturnick and the fertile non-restorer Fielder line. Out of 40 plantlets genotyped from this cross, 6 were wild-type for all 3 NLD mutant alleles. A set of 29 SNP markers that differentiate Arturnick from Fielder were then used to genotype the parental plants (Fielder and each CMS parent used in the cross) and the plantlets from the cross. The 6 plants that only contained wild-type alleles were homozygous for all 29 markers which strongly suggested that these plants are indeed haploid. Final confirmation was obtained by genome-wide genotyping using an 18K SNP Affymetrix array. No significant heterozygosity was observed in any of the 6 plants confirming that they are indeed haploid (Table 4). The haploid induction rate using these triple NLD mutant lines was thus 15% in this experiment (Table 4).

TABLE 4
Summary of Genotyping data from 18K affymetrix chip for progeny
of cross of NLD triple mutant aabbdd lines to a CMS line.

| | Progeny | Haploid |
|---|---|---|
| GENOTYPE NLD Male Parent | Tested | plants |
| a8a8b26b26dN1dN1 | 1 | 0 |
| a8a8b26b26dN2dN2 | 2 | 0 |
| a8a8b26b26dN9dN9 | 2 | 1 |
| a8a8b26b26d8d8 | 1 | 0 |
| a8a8b26b26dN6dN6 | 14 | 1 |
| a8a8b26b26dN7dN7 | 15 | 3 |
| a8a8b26b26dN5dN5 | 5 | 1 |
| Total | 40 | 6 |

### Example 5: Creation of Knockout TaNLD Mutant Lines

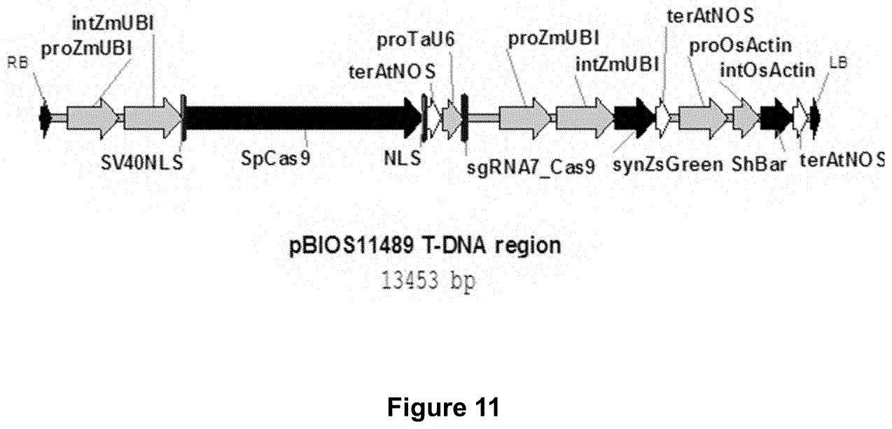

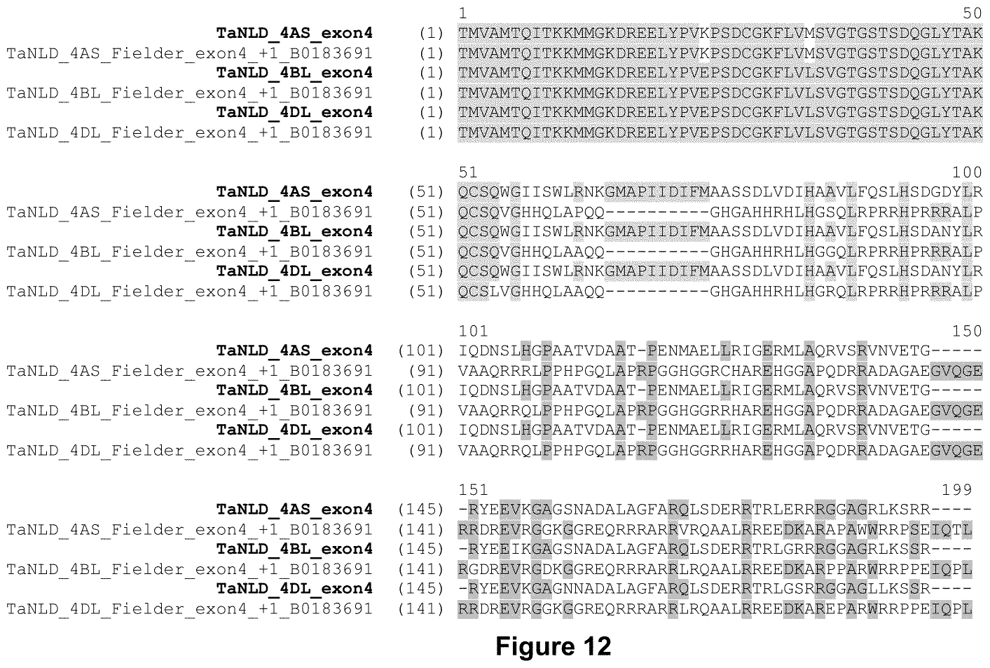

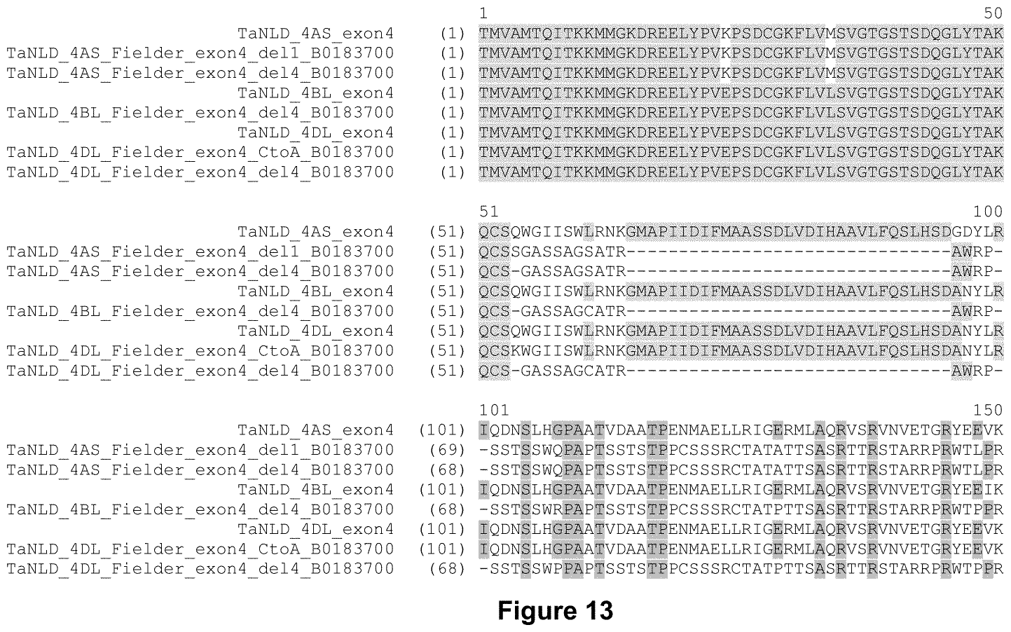

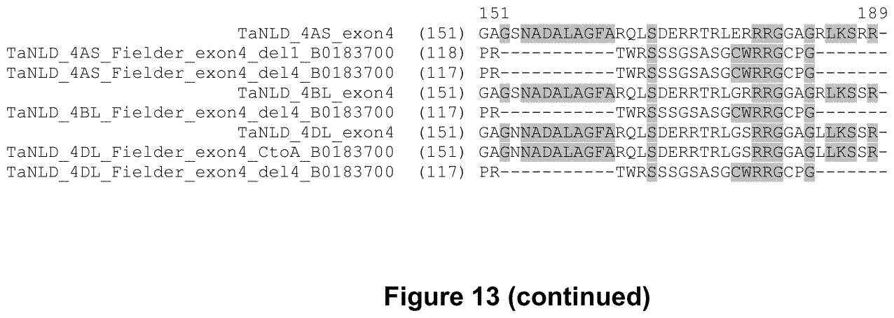

Instead of creating mutations in TaNLD that resemble the ZmNLD-PK6 mutation it is possible to create mutations that eliminate or mutate a larger part or all of the TaNLD genes. These mutations are likely to completely eliminate TaNLD function. A construct was designed to mutate around 134aa of the C-terminus of the TaNLD genes using the Cas9 nuclease from Streptococcus pyogenes. The target site was in a conserved sequence in exon 4. A binary plant transformation construct was made that contains the Cas9 gene with N and C-terminal NLS sequences encoding the protein SEQ ID NO: 48, expressed from the constitutive maize Ubiquitin promoter (SEQ ID NO: 18). The construct also contained a wheat U6 promoter (SEQ ID NO: 19) driving the expression of a gRNA containing the TaNLD-4AS, TaNLD-4BL and TaNLD-4DL target sequence (5′ GGCGAAGCAGTGCTCCCAGT 3′, SEQ ID NO: 82)). In addition, the construct contained a selectable marker gene (BAR) for plant transformation and a visual marker gene (ZsGreen) to aid the detection of transgenic events. FIG. 11 shows a schematic diagram of the T-DNA region (SEQ ID NO: 49). This construct was transferred to the agrobacterial strain EHA105 and transformed into Fielder using a protocol based on immature embryo transformation (Ishida et al.; 2015). The DNA sequence of the regions targeted in Exon4 in transformed plantlets was amplified using primers that amplified all 3 NLD genome copies (Table 1; SEQ ID NO: 78-79). The amplicons obtained were sequenced using Next Generation Sequencing (NGS) and the sequences assigned to genomes based on NLD genome-specific SNPs in the amplicon. Sequence analysis then identified TaNLD mutant T0 plants. Two T0 plants were retained for further analysis. Transformant B0183691 was heterozygous for mutations in each TaNLD-like gene (aAbBdD, SEQ ID NO: 50-52). A protein alignment of TaNLD exon 4 (SEQ ID NO: 58-60) is shown in FIG. 12. Plant B0183700 was heterozygous for mutations in TaNLD-like in genomes A and D and homozygous for a mutation in genome B (aAbbdD, SEQ ID NO: 53-57). A protein sequence alignment of TaNLD exon 4 (SEQ ID NO: 61-65) is shown in FIG. 13. Progeny from these selfed plants are screened to identify combinations of A, B and D genome TaNLD-like mutant T1 plants.

### Example 6: Phenotype of TaNLD-Like Deletion Mutants Obtained with SpCas9

Pollen from homozygote single, double and triple Cas9-derived TaNLD mutants is used to pollinate a CMS wheat line. This wheat line is genetically different to Fielder. Seeds from these crosses are germinated and plantlets genotyped using a panel of markers. Plantlets with a genotype identical to that of the CMS female parent are derived from a haploid induction event. Table 5 shows results from genotyping F1 seed derived from a cross between the T2 progeny of line B0183700 (aabbDD, aabbdD or aabbdd), used as the male parent, and the CMS line Arturnick. The percentage of haploid plants was greatest when the male parent was triple homozygous mutant for TaNLD.

TABLE 5
Summary of Genotyping data from 18K affymetrix
chip for progeny of cross of NLD mutant lines
derived from transformant B0183700 to a CMS line.

| NLD Genotype | Plants | Haploids | % Hapoids |
|---|---|---|---|
| aabbDD | 6 | 0 | 0% |
| aabbdD | 315 | 5 | 2% |
| aabbdd | 87 | 4 | 5% |

### Example 7: Delivery of Genome Editing Tools Via Wheat Nld Haploid Inducer Lines

The wheat nld haploid inducer lines can be used as a vehicle to deliver genome editing (GE) tools into a second genetic background to produce genome-edited mutants directly in that background. In this system (HILAGE or HiEDIT (WO2017004375A1)) GE tools are introduced into the HI line by crossing to a line with the GE tools and selecting for progeny that contain the GE tool and are mutant in the genome A, B and D NLD genes. Alternatively, a HI line can be retransformed with the GE tools. To demonstrate GE delivery from a wheat nld GE line, triple homozygote nld plants identified in example 5 are crossed to the CMS line Arturnick as described in example 6. These plants contain the Cas9 transgene and guide that was used to create the nld mutations in Fielder. The exon4 region from TaNLD4AS SEQ ID NO: 90, TaNLD4BL SEQ ID NO: 91 and TaNLD4DL SEQ ID NO: 92 contains the target sequence (5′ GGCGAAGCAGTGCTCCCAGT 3′, SEQ ID NO: 82). Haploid plants from the progeny of the cross between the Fielder HI line and Arturnick are identified by genotyping as described in example 4. The exon 4 region of the 3 NLD genome copies are amplified from haploids and sequenced. Arturnick haploid plants that have mutations in the NLD genes can then be identified.

### Example 8: Conversion of a Colored Coleoptile Wheat Line to a Haploid Inducer Line

Wheat lines having a colored coleoptile are selected. The selection of these lines is made according to the color of the coleoptile that has to be visible and dominant. To determine if a wheat line has a colored coleoptile, a germination test is made in a growth chamber, ideally a vernalization chamber. The growth conditions are standard conditions for wheat. The coleoptile of the tested wheat line is compared to the coleoptile of a control line having a white/green coleoptile like Apache. Wheat lines having a red coleoptile are easily identified by direct observation. Six such lines, BGA-0664, BGA-0665, BGA-0666, BGA-0667, BGA-0668 and BGA-0669 were identified. BGA-0664, BGA-0665 and BGA-0668 are spring wheats, the others are winter wheats. TaNLD exon4 from the A, B and D genomes were amplified from these lines and sequenced. (SEQ ID NO: 93-110)

TABLE 6
Sequences of NLD genes in different wheat lines

| | TaNLD_4AS_Exon4 | TaNLD_4BL_Exon4 | TaNLD_4DL_Exon4 |
|---|---|---|---|
| BGA-0664 | SEQ ID NO: 93 | SEQ ID NO: 99 | SEQ ID NO: 105 |
| BGA-0665 | SEQ ID NO: 94 | SEQ ID NO: 100 | SEQ ID NO: 106 |
| BGA-0666 | SEQ ID NO: 95 | SEQ ID NO: 101 | SEQ ID NO: 107 |
| BGA-0667 | SEQ ID NO: 96 | SEQ ID NO: 102 | SEQ ID NO: 108 |
| BGA-0668 | SEQ ID NO: 97 | SEQ ID NO: 103 | SEQ ID NO: 109 |
| BGA-0669 | SEQ ID NO: 98 | SEQ ID NO: 104 | SEQ ID NO: 110 |

In all 6 lines the target site for the Cas9 gRNA from example 5 was conserved (5′ GGCGAAGCAGTGCTCCCAGT 3′, SEQ ID NO: 82).

The shoot apical meristem is exposed from colored coleoptile line seeds and bombarded with Cas9 ribonucleoprotein (Cas9 protein and Cas9 gRNA RNA (target SEQ ID NO: 82)) according to the method described by Imai et al. 2020. Plantlets with out of frame mutations in TaNLD genome copies are identified and crossed and/or selfed to obtain progeny that are homozygous for TaNLD knockout mutations in the A, B and D genomes. These aabbdd lines (which are also homozygous for the Rc gene) are used in pollinations as males to females that have green coleoptiles. Progeny of these crosses that possess green rather than colored coleoptiles can be easily visually identified upon germination. These green coleoptile plants are haploids and are treated to double the genome according to well-known procedures (Sood et al; 2003, Niu et al; 2014, Vanous et al. 2017, Hantzschel et al. 2010, Melchinger et al. 2016, Ren 2018, Chaikam et al. 2020) to obtain fertile plants.

### Example 9: Conversion of a Colored Coleoptile Wheat Line to a Haploid Inducer and a Low Palmitic Acid Seed Line

Mutations in the maize FatB Chr6 or FatB chr9 genes reduce the palmitic acid content of maize embryos (Li et al 2011, Zheng et al 2014). Palmitic acid content can thus be used as a marker to identify seeds that contain haploid embryos (or sort isolated embryos into F1 and haploid embryos) if the haploid inducer line contains a FatB loss of function mutation or mutations. F1 embryos will have a reduced Palmitic acid content compared to a haploid embryo. This early haploid marker can be also combined with the coleoptile color marker of example 8 in order to confirm haploids identified on the basis of palmitic acid content.

The maize FatB Chr6 (SEQ ID NO: 111) and Chr9 (SEQ ID NO: 112) protein sequences were used in BLASTP homology searches to identify the wheat homologs in the variety Chinese Spring. Homologs were identified on chromosome 4A (TraesCS4A02G387700) (SEQ ID NO: 113 encoded by SEQ ID NO: 114), 7A (TraesCS7A02G089000) (SEQ ID NO: 115 encoded by SEQ ID NO: 116) and 7D (TraesCS7D02G084400). (SEQ ID NO: 117 encoded by SEQ ID NO: 118). These wheat FatB protein sequences are between 80% to 82% identical to the maize FatB proteins. Primers based on the Wheat FatB Chinese Spring 4A, 7A and 7D genes were used to amplify FatB exon2 genomic sequences (containing the start ATG codon) from the wheat variety Fielder and the 6 colored coleoptile lines in example 8 ((SEQ ID NO: 119 to 139). Exon 2 sequences of TaFatB4A from lines BGA-0664, BGA-0666 and BGA-668 appear to lack 1 nucleotide compared to other sequences which may indicate that in these lines the TaFatB4A copy is inactive.

Two Cas9 gRNAs, g220r and g283r were designed to target 2 regions of TaFatB4A, 7A and 7D exon2 in all the 6 colored coleoptile lines and also in Fielder and Chinese Spring. The targeted sequence for g220r is 5′ TGTCTGAGCCTGTAGTCTTG 3′ SEQ ID NO: 140 and for g283r 5′ GCAAGAAGCATGCTCCAGTC 3′ SEQ ID NO: 141.

The shoot apical meristem is exposed from colored coleoptile line seeds and bombarded with Cas9 ribonucleoprotein (Cas9 protein, Cas9 NLD gRNA (target SEQ ID NO: 82) and Cas9 FATB gRNA RNA (SEQ ID NO: 140, SEQ ID NO: 141)) according to the method described by Imai et al. 2020. Plantlets with out of frame mutations in TaNLD and/or TaFATB genome copies are identified and crossed and/or selfed to obtain progeny that are homozygous for TaNLD knockout mutations in the A, B and D genomes and contain in addition homozygous TaFATB knockout mutations in 1, 2 or 3 genomic loci. These lines are used in pollinations as males to female lines. Seeds with high palmitic acid content in embryos, or isolated embryos with high palmitic acid levels, can be identified with a non-destructive technique such as Near Infra-Red Spectroscopy (NIRS). These high palmitic acid content seeds or isolated embryos are haploids. If the female line has a non-colored coleoptile confirmation of haploidy can be obtained by visualization of the coleoptile color of germinated seeds. Plantlets with green coleoptiles are haploids. Identified haploid embryos or plantlets are treated to double the genome according to well-known procedures (Sood et al; 2003, Niu et al; 2014, Vanous et al. 2017, Hantzschel et al. 2010, Melchinger et al. 2016, Ren 2018, Chaikam et al. 2020) to obtain fertile plants.

### REFERENCES

Alsahlany M, Zarka D, Coombs J and David S. Douches D S (2019) Comparison of Methods to Distinguish Diploid and Tetraploid Potato. Applied Diploid Breeding. American Journal of Potato Research 96, pp 244-254.
Amato F D, Scarascia G T, Monti L M and Bozzini, A (1962) Types and frequencies of chlorophyll mutations in Durum wheat induced by radiations and chemicals. Radiation Botany Volume 2, Issues 3-4, December 1962, Pages 217-239.
Anzalone, A. V., Randolph, P. B., Davis, J. R. et al. Search-and-replace genome editing without double-strand breaks or donor DNA. Nature 576, 149-157 (2019) doi:10.1038/s41586-019-1711-4
Borrino E M and Powell W (1988) Stomatal guard cell length as an indicator of ploidy in microspore-derived plants of barley. Genome, 30(2): 158-160.
Chaikam, V. et al. Analysis of effectiveness of R1-nj anthocyanin marker for in vivo haploid identification in maize and molecular markers for predicting the inhibition of R1-nj expression. Theor. Appl. Genet. 128, 159-171 (2015).
Chaikam V, Martinez L, Melchinger A E, Schipprack W and Boddupalli P M (2016) Development and Validation of Red Root Marker-Based Haploid Inducers in Maize. Crop Sci. DOI: 10.2135/cropsci2015.10.0653
Chaikam, V., Molenaar, W., Melchinger, A. E. & Boddupalli, P. M. Doubled haploid technology for line development in maize: technical advances and prospects. Theor Appl Genet (2019) doi:10.1007/s00122-019-03433-x.
Chaikam, V. et al. Improving the Efficiency of Colchicine-Based Chromosomal Doubling of Maize Haploids. Plants 9, 459 (2020).
Chu C G, Faris J D, Friesen T L, Xu S S. (2006) Molecular mapping of hybrid necrosis genes Ne1 and Ne2 in hexaploid wheat using microsatellite markers. Theor Appl Genet. 112(7):1374-81
Coe E H (1959) A line of maize with high haploid frequency. Am Nat 93: 381-382 El-Hannawy et al, 2011. Production of doubled haploid wheat lines (Triticum aestivum L.) using anther culture technique. Annals of Agricultural Sciences. 56. 63-72.
Faris J D, Zhang Z, Lu H, Lu S, Reddy L, Cloutier S, Fellers J P, Meinhardt S W, Rasmussen J B, Xu S S, Oliver R P, Simons K J, Friesen T L (2010) Proc Natl Acad Sci USA.; 107:13544-9. doi: 10.1073/pnas.1004090107
Ge, W. et al. Maize haploid recognition study based on nuclear magnetic resonance spectrum and manifold learning. Computers and Electronics in Agriculture 170, 105219 (2020).
Geiger et al., Doubled haploids in hybrid maize breeding, Maydica 54(4):485-499 Gilles L M, Khaled A, Laffaire J B, Chaignon S, Gendrot G, Laplaige J, Berges H, Beydon G, Bayle V, Barret P, Comadran J, Martinant J P, Rogowsky P M, Widiez T. (2017) Loss of pollen-specific phospholipase NOT LIKE DAD triggers gynogenesis in maize. EMBO J. 36(6):707-717. doi: 10.15252/embj.201796603.
Gilles et al. Haploid induction in plants, Current Biology 27, R1095-1097, 2017 Hamilton, D. A., Roy, M., Rueda, J. et al. Plant Mol Biol (1992) 18: 211-218
Ho, I Y. Wan Y, Widholm J M and Rayburn A L (1990) The Use of Stomatal Chloroplast Number for Rapid Determination of Ploidy Level in Maize. Plant Breeding 105, pp 203-210. doi.org/10.1111/j.1439-0523.1990.tb01197.x
Häntzschel, K. R. & Weber, G. Blockage of mitosis in maize root tips using colchicine-alternatives. Protoplasma 241, 99-104 (2010).
Hussain et al, 2012. Double Haploid Production in Wheat through Microspore Culture and Wheat×Maize Crossing System: An Overview. ijavms. 6. 10.5455/ijavms.168.
Ishida Y, Tsunashima M, Hiei Y, Komari T (2015) Wheat (Triticum aestivum L.) transformation using immature embryos. Methods Mol Biol. 1223:189-98. doi: 10.1007/978-1-4939-1695-5_15
Kandala et al 2012. Analysis of Moisture Content, Total Oil and Fatty Acid Composition by NIR Reflectance Spectroscopy: A Review. Lecture Notes in Electrical Engineering. 146. 59-80. 10.1007/978-3-642-27638-5_4.
Kelliher et al., 2017; MATRILINEAL, a sperm-specific phospholipase, triggers maize haploid induction. Nature 542: 105-109
Le TDQ, Alvarado C, Girousse C, Legland D, Chateigner-Boutin A L. (2019). Use of X-ray micro computed tomography imaging to analyze the morphology of wheat grain through its development. Plant Methods. 31; 15:84. doi: 10.1186/s13007 0468-y.
Li L, Li H, Li Q, et al. An 11-bp insertion in Zea mays fatb reduces the palmitic acid content of fatty acids in maize grain. PLoS One. 2011; 6(9):e24699. doi:10.1371/journal.pone.0024699
Lin B Y. (1984) Ploidy barrier to endosperm development in maize. Genetics. 107(1):103-15.
Liu, Zhixin & Wang, Yanbo & Ren, Jiaojiao & Mei, Mei & Frei, Ursula & Trampe, Benjamin & Lübberstedt, Thomas & Goldman, I. & Ortiz, Rodomiro. (2016). Maize Doubled Haploids. 10.1002/9781119279723.ch3.
Liu et al., 2017 A 4 bp insertion at ZmPLA1 encoding a putative phospholipase A generates haploid induction in maize, Mol Plant 10: 520-522
Liu et al (2019) Extension of the in vivo haploid induction system from diploid maize to hexaploid wheat. Plant Biotechnol J. 2019 Jul. 25. doi: 10.1111/pbi.13218.
Liu et al. 2019 Efficient induction of haploid plants in wheat by editing of TaMTL using an optimized Agrobacterium-mediated CRISPR system, Journal of Experimental Botany erz529, https://doi.org/10.1093/jxb/erz529
Melchinger, A. E., W. Schipprack, T. Würschum, S. Chen, and F. Technow. (2013). Rapid and accurate identification of in vivo induced haploid seeds based on oil content in maize. Sci. Rep. 3:2129. doi:10.1038/srep02129
Melchinger, A. E., W. Schipprack, H. F. Utz, and V. Mirdita (2014). In vivo haploid induction in maize: Identification of haploid seeds by their oil content. Crop Sci. 54:1497-1504. doi:10.2135/cropsci2013.12.0851
Melchinger, A. E., Molenaar, W. S., Mirdita, V. & Schipprack, W. Colchicine Alternatives for Chromosome Doubling in Maize Haploids for Doubled-Haploid Production. Crop Science 56, 559-569 (2016).
Miao, J, Guo D S, Zhang J Z, Huang Q P, Qin G J, Zhang X, Wan J M, Gu H Y and Qu L J (2013). Targeted mutagenesis in rice using CRISPR-Cas system Cell Res, 23 (10) (2013), pp. 1233-1236
Molenaar, W. S., Oliveira Couto, E. G., Piepho, H. & Melchinger, A. E. Early diagnosis of ploidy status in doubled haploid production of maize by stomata length and flow cytometry measurements. Plant Breed 138, 266-276 (2019).
Nakamura S (2018) Grain dormancy genes responsible for preventing pre-harvest sprouting in barley and wheat. Breeding Science 68: 295-304. doi:10.1270/jsbbs.17138
Niu, Z. et al. Review of doubled haploid production in durum and common wheat through wheat×maize hybridization. Plant Breed 133, 313-320 (2014).)
Pukhal′skĩĩ V A, Martynov S P, Bilinskaia E N. (2010) Dynamics of hybrid necrosis genes in Russian cultivars of common wheat (Triticum aestivum L.). Genetika. 46(11):1516-24.
Ren, X. Doubling Effect of Anti-microtubule Herbicides on the Maize Haploid. Emir J Food Agric 903 (2018) doi:10.9755/ejfa.2018.v30.i10.1828.
Röber et al., In vivo haploid induction in maize—Performance of new inducers and significance of doubled haploid lines in hybrid breeding Maydica, 50(3-4): 275-283, 2005
Rousseau D, Widiez T, Di Tommaso S, Rositi H, Adrien J, Maire E, Langer M, Olivier C, Peyrin F, Rogowsky P (2015). Fast virtual histology using X-ray in-line phase tomography: application to the 3D anatomy of maize developing seeds. Plant Methods. 18; 11:55. doi: 10.1186/s13007-015-0098-y.
Sari N, Abak K and Pitrat M (1999) Comparison of ploidy level screening methods in watermelon: Citrullus lanatus (Thunb.) Matsum. and Nakai. Scientia Horticulturae 82, 3-4, pp 265-277. https://doi.org/10.1016/S0304-4238(99)00077-1.
See P T, lagallo E M, Oliver R P, Moffat C S. (2019) Heterologous Expression of the Pyrenophora tritici-repentis Effector Proteins ToxA and ToxB, and the Prevalence of Effector Sensitivity in Australian Cereal Crops. Front Microbiol. 10:182. doi: 10.3389/fmicb.2019.00182.
Shi G, Zhang Z, Friesen T L, Raats D, Fahima T, Brueggeman R S, Lu S, Trick H N, Liu Z, Chao W, Frenkel Z, Xu S S, Rasmussen J B, Faris J D. (2016) The hijacking of a receptor kinase-driven pathway by a wheat fungal pathogen leads to disease. Sci Adv. 2(10):e1600822.
Shoeva O Y and Khlestkina E K (2015). The specific features of anthocyanin biosynthesis regulation in wheat. In Y. Ogihara et al. (eds.), Advances in Wheat Genetics: From Genome to Field, DOI 10.1007/978-4-431-55675-6_16
Sood, S., Dhawan, R., Singh, K. & Bains, N. S. Development of novel chromosome doubling strategies for wheat×maize system of wheat haploid production. Plant Breeding 122, 493-496 (2003).
W. Tadesse, S. Tawkaz, M. N. Inagaki, E. Picard, and M. Baum. 2013. Methods and Applications of Doubled Haploid Technology in Wheat Breeding. ICARDA, Aleppo, Syria. 36 pp.
Twell, D., Patel, S., Sorensen, A. et al. Sexual Plant Reprod (1993) 6: 217-224
Vanous, K., Vanous, A., Frei, U. K. & Lübberstedt, T. Generation of Maize (Zea mays) Doubled Haploids via Traditional Methods: Generation of Maize Doubled Haploids. in Current Protocols in Plant Biology (eds. Stacey, G. et al.) 147-157 (John Wiley & Sons, Inc., 2017). doi:10.1002/cppb.20050.
Vikas V K, Tomar SMS, Sivasamy M, Kumar J, Jayaprakash P, Kumar A, Peter J, Nisha R, Punniakotti E (2013) Hybrid necrosis in wheat: evolutionary significance or potential barrier for gene flow? Euphytica 194, Issue 2, pp 261-275.
Würschum T, Langer S M, Longin C F. (2015) Genetic control of plant height in European winter wheat cultivars. Theor Appl Genet. 2015 May; 128(5):865-74. doi: 10.1007/s00122-015-2476-2.
Yao et al, 2018 Nat Plants. 2018 August; 4(8):530-533.
Ye G J, Wei L, Chen W J, Zhang B, Liu B L and Zhang H G (2017) Frame-shift Mutation Causes the Function Loss of TaMYB-A1 Regulating Anthocyanin Biosynthesis in Triticum aestivum. Cereal Research Communications 45(1), pp. 35-46 (2017) DOI: 10.1556/0806.44.2016.042
Zetsche B, Gootenberg J S, Abudayyeh 00, Slaymaker I M, Makarova K S, Essletzbichler P, Volz S E, Joung J, van der Oost J, Regev A, Koonin E V, Zhang F (2015). Cpf1 is a single RNA-guided endonuclease of a class 2 CRISPR-Cas system. Cell. 163(3):759-71. doi: 10.1016/j.cell.2015.09.038.
Zhang P, Hiebert C W, McIntosh R A, McCallum B D, Thomas J B, Hoxha S, Singh D, Bansal U. (2016) The relationship of leaf rust resistance gene Lr13 and hybrid necrosis gene Ne2m on wheat chromosome 2B S. Theor Appl Genet. 129(3):485-93. doi: 10.1007/s00122-015-2642-6
Zheng et al 2014, Theor Appl Genet. 2014 July; 127(7):1537-47
Zhong et al. Mutation of ZmDMP enhances haploid induction in maize, Nature Plants, Vol. 5, 2019

1. A wheat haploid inducer plant comprising at least one cell which presents inhibition of expression of three NLD genes of genomes A, B and D, wherein the NLD genes of genomes A, B and D present at least 95% identity with SEQ ID NO: 3, 4 and 9 respectively, and at least one dominant or semi-dominant genetic marker, wherein the genetic marker produces, by itself or in complementation with another gene, a phenotype that can be detected.

2. The wheat haploid inducer plant of claim 1, wherein the plant comprises at least two different genetic markers from two different marker systems.

3. The wheat haploid inducer plant of claim 1, wherein the genetic marker is selected from a dominant or semi-dominant visual genetic marker, a gene modifying morphology of the plant, a genetic marker producing a phenotype when combined with another genetic marker, and an inducible genetic marker.

4. The wheat haploid inducer plant of claim 1 comprising at least a mutation in one of the NLD genes of genomes A, B and D that results in a frameshift in a coding sequence.

5. The wheat haploid inducer plant of claim 4, wherein the frameshift is in exon 4 of the NLD gene.

6. The wheat haploid inducer plant of claim 1, wherein inhibition of the expression of the NLD genes has been is obtained by site directed mutagenesis, chemical mutagenesis, physical mutagenesis of the genes, and/or introduction of a RNAi construct against the NLD genes in the genome of the plant.

7. A method for identifying the wheat haploid inducer plant of claim 1 comprising detecting mutations of the NLD genes, and/or presence of a vector inhibiting expression of the NLD genes, and the presence of the dominant or semi-dominant genetic marker, which is able to produce by itself or in complementation with another gene, a phenotype that can be detected, in the A, B or D genomes of a wheat plant.

8. A method for quality control of seed lots comprising wheat haploid inducer lines according to claim 1 comprising:
(a) taking a sample of seeds from a seed lot comprising wheat haploid inducer lines;
(b) conducting molecular analyses to identify and quantify a presence of haploid inducer or non-inducer alleles, and of the genetic marker;
(c) deducing from (b) a genetic purity value of the lot for haploid inducer character.

9. A method for obtaining the plant of claim 1 comprising
(a) introducing into a genome of at least one cell of a wheat plant at least one mutation in one NLD gene of one of the A, B or D genomes and/or a genetic construct inhibiting expression of one NLD gene leading to a plant having a modified genome, and presenting inhibition of the NLD genes on the A, B and D genomes, and
(b) introducing at least one genetic marker system in the genome of a cell of the wheat plant if the marker is not already present, and
(c) obtaining a wheat plant comprising at least one cell which presents inhibition of the expression of the three NLD genes of its genomes and the genetic marker system.

10. A method for inducing haploid progeny comprising pollinating a female wheat plant with the wheat plant of claim 1.

11. The wheat haploid inducer plant of claim 1, further comprising in its genome one or more expression cassettes comprising at least one gene encoding for a nuclease capable of modifying the genome.

12. The wheat haploid inducer plant of claim 11, wherein the nuclease is a CRISPR-Cas, and wherein the plant further comprises an expression cassette comprising a polynucleotide targeting one or several specific loci of interest in the wheat plant's genome to induce a CRISPR-Cas-mediated genome modification.

13. A method for genetically modifying a genome of a wheat plant comprising pollinating a second plant with pollen of the wheat haploid inducer plant of claim 11.

14. A method for identifying a haploid wheat plant within a wheat plant population comprising selecting a plant from the wheat plant population which does not present a phenotype associated with the genetic marker, wherein the wheat plant population consists of plants obtained after cross of the wheat haploid inducer plant of claim 1 as a pollen provider and of another wheat plant as a female plant.

15. The wheat haploid inducer plant of claim 3 comprising at least a mutation in one of the NLD genes of genomes A, B and D that results in a frameshift in a coding sequence.

16. The wheat haploid inducer plant of claim 15, wherein the frameshift is in exon 4 of the NLD gene.

17. The wheat haploid inducer plant of claim 16, wherein inhibition of the expression of the NLD genes is obtained by site directed mutagenesis, chemical mutagenesis, physical mutagenesis of the genes, and/or introduction of a RNAi construct against the NLD genes in the genome of the plant.

18. The wheat haploid inducer plant of claim 17, further comprising in its genome one or more expression cassettes comprising at least one gene encoding for a nuclease capable of modifying the genome.

19. The wheat haploid inducer plant of claim 18, wherein the nuclease is a CRISPR-Cas, and wherein the plant further comprises an expression cassette comprising a polynucleotide targeting one or several specific loci of interest in a genome of the wheat plant to induce a CRISPR-Cas-mediated genome modification.

20. The wheat haploid inducer plant of claim 1, wherein the genetic marker is selected from a gene involved in anthocyanin biosynthesis, a component of a system inducing hybrid necrosis when combined and a gene inducing pre-harvest sprouting in specific conditions.


SEQUENCE LISTING (141 sequences)

SEQ ID NO: 1 - DNA, 1892 bp, Zea mays
agttcatcactaatcacacttattgtgccctcgacgagtatctatagctagctcattaatcgattcgggggtgtgttgtcgaaggcggcaatggcgagctactcgtcgcggcgtccatgcaatacctgtagcacgaaggcgatggccgggagcgtggtcggcgagcccgtcgtgctggggcagagggtgacggtgctgacggtggacggcggcggcgtccggggtctcatcccgggaaccatcctcgccttcctggaggccaggctgcaggagctggacggaccggaggcgaggctggcggactacttcgactacatcgccggaaccagcaccggcggtctcatcaccgccatgctcaccgcgcccggcaaggacaagcggcctctctacgctgccaaggacatcaaccacttttacatgcagaactgcccgcgcatctttcctcagaagtgagtccgatgctgccgccattgttcttgcatccatccagcatcgtacgtacgtcctctatacatctgcggatcatcatgtgcgcatgtttgtggcatgcatgcatgcatgtgagcaggagcaggcttgcggccgccatgtccgcgctgaggaagccaaagtacaacggcaagtgcatgcgcagcctgattaggagcatcctcggcgagacgagggtaagcgagacgctgaccaacgtcatcatccctgccttcgacatcaggctgctgcagcctatcatcttctctacctacgacgtacgtacgtcgtcacgaatgattcatctgtacgtcgtcgcatgcgaatggctgcctacgtacgccgtgcgctaacatactcagctctttcctatctgctgcgccaatttgcaggccaagagcacgcctctgaagaacgctctgctctcggacgtgtgcattggcacgtccgccgcgccgacctacctcccggcgcactacttccagactgaagacgccaacggcaaggagcgcgaatacaacctcatcgacggcggtgtggcggccaacaacccggtaactgactagctaactggaaaacggacgcacagactccatgtccatggcggcccacaaggtcgatgctaattgttgcttatgtatgtcgcccgattgcacatgcgtagacgatggttgcgatgacgcagatcaccaaaaagatgcttgccagcaaggacaaggccgaggagctgtacccagtgaagccgtcgaactgccgcaggttcctggtgctgtccatcgggacggggtcgacgtccgagcagggcctctacacggcgcggcagtgctcccggtggggtatctgccggtggctccgcaacaacggcatggcccccatcatcgacatcttcatggcggccagctcggacctggtggacatccacgtcgccgcgatgttccagtcgctccacagcgacggcgactacctgcgcatccaggacaactcgctccgtggcgccgcggccaccgtggacgcggcgacgccggagaacatgcggacgctcgtcgggatcggggagcggatgctggcacagagggtgtccagggtcaacgtggagacagggaggtacgaaccggtgactggcgaaggaagcaatgccgatgccctcggtgggctcgctaggcagctctccgaggagaggagaacaaggctcgcgcgccgcgtctctgccatcaacccaagaggctctagatgtgcgtcgtacgatatctaagacaagtggctttactgtcagtcacatgcttgtaaataagtagactttattttaataaaacataaaaatatatatatgttcttgaatataaaattgataaccaaattaaaattcgaaccatcacttatacataattttactttattttttataaaacgtgaacgggaaggac

SEQ ID NO: 2 - protein, 428 aa, Zea mays
MASYSSRRPCNTCSTKAMAGSVVGEPVVLGQRVTVLTVDGGGVRGLIPGTILAFLEARLQELDGPEARLADYFDYIAGTSTGGLITAMLTAPGKDKRPLYAAKDINHFYMQNCPRIFPQKSRLAAAMSALRKPKYNGKCMRSLIRSILGETRVSETLTNVIIPAFDIRLLQPIIFSTYDAKSTPLKNALLSDVCIGTSAAPTYLPAHYFQTEDANGKEREYNLIDGGVAANNPTMVAMTQITKKMLASKDKAEELYPVKPSNCRRFLVLSIGTGSTSEQGLYTARQCSRWGICRWLRNNGMAPIIDIFMAASSDLVDIHVAAMFQSLHSDGDYLRIQDNSLRGAAATVDAATPENMRTLVGIGERMLAQRVSRVNVETGRYEPVTGEGSNADALGGLARQLSEERRTRLARRVSAINPRGSRCASYDI

SEQ ID NO: 3 - DNA, 1900 bp, Triticum aestivum
cgatgatggcaagctactggtgccggcggccctgcgagtcgtgcagcacgcgggcgatggcgggcagcgtggtcggccagccggtggcgccggggcagcgggtgacggtgctgaccatcgacgggggcggcatccgcggcatcatcccgggcaccatcctcgacttcctcgaggccaagctgcaggagctggacggcccgggcgcgcgcctggccgactacttcgactgcatcgccggcaccagcaccggcggcctcatcaccgccatgatcaccgcgcccggcaaggacggccgcccgctcttcgccgccagggacgtcaaccgcttctacctcgacaacgggccctacatcttccctcaaaggcgagagcacgaacgatctcatctcatggacatggatcatgcgagctgaactggtggttgatgtgtggattgtgttgagctgtgccaggaggtgcgcgctcgccgcggtgaccgcatcgctgaggcggccgaggtacagcggcaagtacctgcacggcaagatcaggagcatgctcggcgagacgaggctgtgcgacgcgctcaccgacgtcgtcatccccaccttcgacgtcaagcttctccagcccatcatcttctccacatacgacgtatatatgctaattttatacttggatcatggatcagacagacatggggctaaaaacgtgatggatcatgtgtgcgtgcgtgcaggccaagagcatgcccctgaagaacgcgcggctcgccgacatctgcatcggcacctccgccgccccgacctacctccccgcgcaccacttccacacccacgacggcaacggcaaggagcgcgagtacaacctcatcgacggcggcgtcgccgccaacaatccggtaaccaatcaagcgtctgtccgtctgtcagatgttcagacacgcttgcccgacccgatcacactgatgaactgagctgtgacaaacgcagacgatggtggcgatgacgcagatcaccaagaagatgatgggcaaggacagggaggagctgtacccggtgaagccgtcggactgcggcaagttcctggtgatgtccgtcggcaccggctcgacgtccgaccaggggctgtacacggcgaagcagtgctcccagtggggcatcatcagctggctccgcaacaagggcatggcgcccatcatcgacatcttcatggcagccagctccgacctcgtcgacatccacgccgccgtgctcttccagtcgctgcacagcgacggcgactacctccgcatccaggacaactcgctccacggcccggcggccacggtggacgctgccacgcccgagaacatggcggagctcctcaggatcggcgagcggatgctggcgcagagggtgtccagggtgaacgtcgagaccgggaggtacgaggaggtaaagggggccgggagcaacgccgacgcgctcgccgggttcgccaggcagctctccgacgagaggaggacaaggctcgagcgccggcgtggtggcgccggccgtctgaaatccagacgctgaccttgtagcttagcgtggcttagtttcatttattttctatataaatttttggcggggatagatagagaaagcttttcacatggataatgaaaggaaatgatgagctcaagaaaaataacaacacttttttttgcgtagtcaagaaaaaataacaagacttgatgaaaccaaaatgcctctcaattggtcaccatggcaattgtatatccctttcgacatcatcatcatgaataaatcaacagtgaacaagagagcaaacgaccaacaaaaatttacacaaagcctcctacctatttcggcaagcttcaaaataaatgaatggattgcagcatagaaagaaaagaaaaacctgacctatacaa

SEQ ID NO: 4 - DNA, 1940 bp, Triticum aestivum
cgatcggacgcgtggccgccgggttcaatcgatggcaagctactggggccggcgaccctgcgagtcgtgcagcacgagggcgatggcgggcagcgtggtgggccagccggtggcgccggggcagcgggtgacggtgctgaccatcgacgggggcggcatccgcggcatcatcccgggcaccatcctcgccttcctcgaggccaagctgcaggagctggacgggccgggcgcgcgcctggccgactacttcgactgcatcgccggcaccagcaccggcggcctcatcaccgccatgatcaccgcgcccggcaaggacggctgcccgctcttcgccgccagggacgtcaaccgcttctacctcgacaatggcccctacatcttcccgcaaaggtgagagcgcgaacgatctcatctcatggacatggatcgtgcgagctgaactggtgattgatgtatgtattgcattgagttgtgccaggaggtgcgcgctggccgcggtgaccgcgtcgctgaggcggccgaggtacaacggcaagtacctgcacgggaagatcaggagcatgctcggcgagacgaggctgtccgacgcgctcaccgacgtggtcatccccaccttcgacgtcaagcttctccagcccatcatcttctccacatacgacgtatgccaattttatatgtataagagaactaatgtgatgatcagatagatccatccttggatcatggatcagacagacatggggctaaaaatgtgatggatcacgcgtgcgtgcgtgcaggccaagagcatgcccctgaagaacgcgcgactcgccgacgtgtgcatcggcacctccgccgctccgacctacctccccgcgcaccacttccacacccacgacggcaacggcaaggagcgcgagtacaacctcatcgacggcggcgtcgccgccaacaatccggtaacgaatcaagcctctgtccgtcggtcagatgttcagacacgcttgcccgacccgagcacactgatgaactgagccgtgagaaatgcagacgatggtggcgatgacgcagatcaccaagaagatgatgggcaaggacagggaggagctgtacccggtggagccgtcggactgcggcaagttcctggtgctgtccgtcgggaccggctcgacgtccgaccaggggctgtacacggcgaagcagtgctcccagtggggcatcatcagctggctgcgcaacaagggcatggcgcccatcatcgacatcttcatggccgccagctccgacctcgtcgacatccacgccgccgtgctcttccagtcgctgcacagcgacgccaactacctccgcatccaggacaactcgctccacggcccggcggccacggtggacgccgccacgcccgagaacatggcggagctcctcaggatcggcgagcggatgctggcgcagagggtgtccagggtgaacgtcgagaccgggaggtacgaggaggtaaagggggccgggaacaacgccgacgcgctcgccggcttcgccaggcagctctccgacgagaggaggacaaggctcgggagccggcgcggtggcgccggccgcctgaaatccagccgctgatcttgtagcttagtgtggcttagtttcatttattttctatatacagtatatttttttggctgggataggtagagaaagcttcacatggataatggatggaaatgaggagtttaggaaaaataacaacacttaatgaaaccaaaatgtctctcggttgttcaccgtggcaattgcatatcccttccgatataatcatgaatttaatcaacagtgaacaatagcgcaaacgacgaacaaaaatttacacaaagcctcctccctattttggcaagcttcaaaataaatgaatggattgcagaatagaaagaagaagaaaaacctgggctatacaacagaacact

SEQ ID NO: 5 - DNA, 1400 bp, Triticum aestivum
tgaggcggccgaggtacaacggcaagtacctgcacggcaagatcaggagcatgctcggcgagacgaggctgtgcgacgcgctcaccgacgtcgtcatccccaccttcgacgtcaagcttctccagcccatcatcttctccacatacgacgtatgctaattttatacgaggactaatgtgatgatcagatcgatccatccttggatcatggatcagacagacatggggctaaaaacgtgatggatcatgcgtgcgtcgtccgtgcaggccaggtgcatgcccctgaagaacgcgcggctcgccgacgtctgcatcggcacctcggccgctccgacctgcctccccgcgcaccacttccacacccacgacggcaacggcaaggagcgtgagtacaacctcatcgacggtggcgtcgccgccaacaatccggtaaccaatcaagcgtctgcccgtcggtcagatgttcagacacgcttgcccgacccgagcacactgaccaactgagctgtgagaaatgcagacgatggtggcgatgacgcagatcaccaagaagatgatgggcaaggacagggaggagctgtacccggtggagccgtcggactgcggcaagttcctggtgctgtccgtcgggaccggctcgacgtccgaccaggggctgtacacggcgaagcagtgctcccagtggggcatcatcagctggctgcgcaacaagggcatggcgcccatcatcgacatcttcatggcggccagctccgacctcgtcgacatccacgccgccgtgctcttccagtcgctgcacagcgacgccaactacctccgcatccaggacaactcgctccacggcccggcggccacggtggacgccgccacgcccgagaacatggcggagctcctcaggatcggcgagcggatgctggcgcagagggtgtccagggtgaacgtggagaccgggaggtacgaggagataaagggggccgggagcaacgccgacgcgctcgccggcttcgccaggcagctctccgacgagaggaggacaaggctcggccgccggcgcggtggcgccggccgcctgaaatccagccgctgatcttgtagcttagtgtggcttagtttcattcattttctatatacagtatatctttttggctgggataggtagagaaagcttcacatggataatgacaggaaatgaggagttctggaaaaataacaacacttgatggaaccaaaatgtctctcaattggtcaccgtggcaattgtatatcccttccgatatcatcatgaatttaatcaccggtgaacaatagcgcaaacgaccaacaaaaatttacacacagcatccctactttggctagcttgaaaataaatgaatggattgcagcatagaaagaaaaaagaaa

SEQ ID NO: 6 - protein, 421 aa, Triticum aestivum
MASYWCRRPCESCSTRAMAGSVVGQPVAPGQRVTVLTIDGGGIRGIIPGTILDFLEAKLQELDGPGARLADYFDCIAGTSTGGLITAMITAPGKDGRPLFAARDVNRFYLDNGPYIFPQRCALAAVTASLRRPRYSGKYLHGKIRSMLGETRLCDALTDVVIPTFDVKLLQPIIFSTYDAKSMPLKNARLADICIGTSAAPTYLPAHHFHTHDGNGKEREYNLIDGGVAANNPTMVAMTQITKKMMGKDREELYPVKPSDCGKFLVMSVGTGSTSDQGLYTAKQCSQWGIISWLRNKGMAPIIDIFMAASSDLVDIHAAVLFQSLHSDGDYLRIQDNSLHGPAATVDAATPENMAELLRIGERMLAQRVSRVNVETGRYEEVKGAGSNADALAGFARQLSDERRTRLERRRGGAGRLKSRR

SEQ ID NO: 7 - protein, 421 aa, Triticum aestivum
MASYWGRRPCESCSTRAMAGSVVGQPVAPGQRVTVLTIDGGGIRGIIPGTILAFLEAKLQELDGPGARLADYFDCIAGTSTGGLITAMITAPGKDGCPLFAARDVNRFYLDNGPYIFPQRCALAAVTASLRRPRYNGKYLHGKIRSMLGETRLSDALTDVVIPTFDVKLLQPIIFSTYDAKSMPLKNARLADVCIGTSAAPTYLPAHHFHTHDGNGKEREYNLIDGGVAANNPTMVAMTQITKKMMGKDREELYPVEPSDCGKFLVLSVGTGSTSDQGLYTAKQCSQWGIISWLRNKGMAPIIDIFMAASSDLVDIHAAVLFQSLHSDANYLRIQDNSLHGPAATVDAATPENMAELLRIGERMLAQRVSRVNVETGRYEEVKGAGNNADALAGFARQLSDERRTRLGSRRGGAGRLKSSR

SEQ ID NO: 8 - protein, 291 aa, Triticum aestivum
RRPRYNGKYLHGKIRSMLGETRLCDALTDVVIPTFDVKLLQPIIFSTYDARCMPLKNARLADVCIGTSAAPTCLPAHHFHTHDGNGKEREYNLIDGGVAANNPTMVAMTQITKKMMGKDREELYPVEPSDCGKFLVLSVGTGSTSDQGLYTAKQCSQWGIISWLRNKGMAPIIDIFMAASSDLVDIHAAVLFQSLHSDANYLRIQDNSLHGPAATVDAATPENMAELLRIGERMLAQRVSRVNVETGRYEEIKGAGSNADALAGFARQLSDERRTRLGRRRGGAGRLKSSR

SEQ ID NO: 9 - DNA, 1565 bp, Triticum aestivum
atggcaagctactggtgccggcgcccctgcgagtcgtgcagcacgagggcgatggcgggcagcgtggtgggccagccggtggctccggggcagcgggtgacggtgctgaccatcgacgggggcggcatccgcggcctcatcccgggcaccatcctcgccttcctcgaggccaagctgcaggagctggacgggccgggcgcgcgcctggccgactacttcgactgcatcgccggcaccagcaccggcggcctcatcaccgccatgatcaccgcgcccggcaaggacggccgcccgctcttcgccgccaaggacgtcaaccgcttctacctcgacaacgggccctacatcttcccgcaaaggtgagagcacgaacgatctcatctcatggacatggatcatgcgcgctgaactggtgattgatgtatgaattgcattgagttgtgccaggaggtgcgcgctcgccgcggtgaccgcgtcgctgaggcggccgaggtacaacggcaagtacctgcacggcaagatcaggagcatgctcggcgagacgaggctgtgcgacgcgctcaccgacgtcgtcatccccaccttcgacgtcaagcttctccagcccatcatcttctccacatacgacgtatgctaattttatacgaggactaatgtgatgatcagatcgatccatccttggatcatggatcagacagacatggggctaaaaacgtgatggatcatgcgtgcgtcgtccgtgcaggccaggtgcatgcccctgaagaacgcgcggctcgccgacgtctgcatcggcacctcggccgctccgacctgcctccccgcgcaccacttccacacccacgacggcaacggcaaggagcgtgagtacaacctcatcgacggtggcgtcgccgccaacaatccggtaaccaatcaagcgtctgcccgtcggtcagatgttcagacacgcttgcccgacccgagcacactgaccaactgagctgtgagaaatgcagacgatggtggcgatgacgcagatcaccaagaagatgatgggcaaggacagggaggagctgtacccggtggagccgtcggactgcggcaagttcctggtgctgtccgtcgggaccggctcgacgtccgaccaggggctgtacacggcgaagcagtgctcccagtggggcatcatcagctggctgcgcaacaagggcatggcgcccatcatcgacatcttcatggcggccagctccgacctcgtcgacatccacgccgccgtgctcttccagtcgctgcacagcgacgccaactacctccgcatccaggacaactcgctccacggcccggcggccacggtggacgccgccacgcccgagaacatggcggagctcctcaggatcggcgagcggatgctggcgcagagggtgtccagggtgaacgtggagaccgggaggtacgaggagataaagggggccgggagcaacgccgacgcgctcgccggcttcgccaggcagctctccgacgagaggaggacaaggctcggccgccggcgcggtggcgccggccgcctgaaatccagccgctga

SEQ ID NO: 10 - protein, 421 aa, Triticum aestivum
MASYWCRRPCESCSTRAMAGSVVGQPVAPGQRVTVLTIDGGGIRGLIPGTILAFLEAKLQELDGPGARLADYFDCIAGTSTGGLITAMITAPGKDGRPLFAAKDVNRFYLDNGPYIFPQRCALAAVTASLRRPRYNGKYLHGKIRSMLGETRLCDALTDVVIPTFDVKLLQPIIFSTYDARCMPLKNARLADVCIGTSAAPTCLPAHHFHTHDGNGKEREYNLIDGGVAANNPTMVAMTQITKKMMGKDREELYPVEPSDCGKFLVLSVGTGSTSDQGLYTAKQCSQWGIISWLRNKGMAPIIDIFMAASSDLVDIHAAVLFQSLHSDANYLRIQDNSLHGPAATVDAATPENMAELLRIGERMLAQRVSRVNVETGRYEEIKGAGSNADALAGFARQLSDERRTRLGRRRGGAGRLKSSR

SEQ ID NO: 11 - DNA, 567 bp, Triticum aestivum
acgatggtggcgatgacgcagatcaccaagaagatgatgggcaaggacagggaggagctgtacccggtgaagccgtcggactgcggcaagttcctggtgatgtccgtcggcaccggctcgacgtccgaccaggggctgtacacggcgaagcagtgctcccagtggggcatcatcagctggctccgcaacaagggcatggcgcccatcatcgacatcttcatggcagccagctccgacctcgtcgacatccacgccgccgtgctcttccagtcgctgcacagcgacggcgactacctccgcatccaggacaactcgctccacggcccggcggccacggtggacgctgccacgcccgagaacatggcggagctcctcaggatcggcgagcggatgctggcgcagagggtgtccagggtgaacgtcgagaccgggaggtacgaggaggtaaagggggccgggagcaacgccgacgcgctcgccgggttcgccaggcagctctccgacgagaggaggacaaggctcgagcgccggcgtggtggcgccggccgtctgaaatccagacgctga

SEQ ID NO: 12 - DNA, 567 bp, Triticum aestivum
acgatggtggcgatgacgcagatcaccaagaagatgatgggcaaggacagggaggagctgtacccggtggagccgtcggactgcggcaagttcctggtgctgtccgtcgggaccggctcgacgtccgaccaggggctgtacacggcgaagcagtgctcccagtggggcatcatcagctggctgcgcaacaagggcatggcgcccatcatcgacatcttcatggccgccagctccgacctcgtcgacatccacgccgccgtgctcttccagtcgctgcacagcgacgccaactacctccgcatccaggacaactcgctccacggcccggcggccacggtggacgccgccacgcccgagaacatggcggagctcctcaggatcggcgagcggatgctggcgcagagggtgtccagggtgaacgtcgagaccgggaggtacgaggaggtaaagggggccgggaacaacgccgacgcgctcgccggcttcgccaggcagctctccgacgagaggaggacaaggctcgggagccggcgcggtggcgccggcctcctgaaatccagccgctga

SEQ ID NO: 13 - DNA, 567 bp, Triticum aestivum
acgatggtggcgatgacgcagatcaccaagaagatgatgggcaaggacagggaggagctgtacccggtggagccgtcggactgcggcaagttcctggtgctgtccgtcgggaccggctcgacgtccgaccaggggctgtacacggcgaagcagtgctcccagtggggcatcatcagctggctgcgcaacaagggcatggcgcccatcatcgacatcttcatggcggccagctccgacctcgtcgacatccacgccgccgtgctcttccagtcgctgcacagcgacgccaactacctccgcatccaggacaactcgctccacggcccggcggccacggtggacgccgccacgcccgagaacatggcggagctcctcaggatcggcgagcggatgctggcgcagagggtgtccagggtgaacgtggagaccgggaggtacgaggagataaagggggccgggagcaacgccgacgcgctcgccggcttcgccaggcagctctccgacgagaggaggacaaggctcggccgccggcgcggtggcgccggccgcctgaaatccagccgctga

SEQ ID NO: 14 - protein, 188 aa, Triticum aestivum
TMVAMTQITKKMMGKDREELYPVKPSDCGKFLVMSVGTGSTSDQGLYTAKQCSQWGIISWLRNKGMAPIIDIFMAASSDLVDIHAAVLFQSLHSDGDYLRIQDNSLHGPAATVDAATPENMAELLRIGERMLAQRVSRVNVETGRYEEVKGAGSNADALAGFARQLSDERRTRLERRRGGAGRLKSRR

SEQ ID NO: 15 - protein, 188 aa, Triticum aestivum
TMVAMTQITKKMMGKDREELYPVEPSDCGKFLVLSVGTGSTSDQGLYTAKQCSQWGIISWLRNKGMAPIIDIFMAASSDLVDIHAAVLFQSLHSDANYLRIQDNSLHGPAATVDAATPENMAELLRIGERMLAQRVSRVNVETGRYEEVKGAGNNADALAGFARQLSDERRTRLGSRRGGAGLLKSSR

SEQ ID NO: 16 - protein, 188 aa, Triticum aestivum
TMVAMTQITKKMMGKDREELYPVEPSDCGKFLVLSVGTGSTSDQGLYTAKQCSQWGIISWLRNKGMAPIIDIFMAASSDLVDIHAAVLFQSLHSDANYLRIQDNSLHGPAATVDAATPENMAELLRIGERMLAQRVSRVNVETGRYEEIKGAGSNADALAGFARQLSDERRTRLGRRRGGAGRLKSSR

SEQ ID NO: 17 - protein, 1273 aa, Artificial
  LbCpf1_NLS-HA
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHKRPAATKKAGQAKKKKGSYPYDVPDYAYPYDVPDYAYPYDVPDYA

SEQ ID NO: 18 - DNA, 1994 bp, Zea mays
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggt

SEQ ID NO: 19 - DNA, 362 bp, Triticum aestivum
gaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagactt

SEQ ID NO: 20 - DNA, 13040 bp, Artificial
  Construct pBIOS11170 - T-DNA region
aaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctaattaattaggctagaggataactataacggtcctaaggtagcgatacgcatcgtatcggagtctcagcttacgcttgtagactgatccgtctcacgagtctagacgttctaggtcactgacgtcagtctgttggaaggcacgatcgctgagtgtctaatccaaggttctatagcccagacctacgtgcaactggacttgttgacgtctcgttgagtgcacctatcgacaggcactagcgacgcacgacgtaacctgatacgaggcttaattaattagggagaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgtaatttctactaagtgtagatcctcctcgtacctcccggtctcgtttttttgtgctctcttaaggtagcggagaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgtaatttctactaagtgtagattctcctcgtacctcccggtctcctttttttgtgtggcaaacagctattatgggtattatgggtggagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtctccaccatgagcaagctggagaagttcaccaactgctactccctgagcaagaccctcaggttcaaggccatcccagtgggcaagacccaagagaacatcgacaacaagaggctgctcgtggaggacgagaagagggccgaggactacaagggcgtgaagaagctcctggaccgctactacctgtccttcatcaacgacgtcctgcactccatcaagctcaagaacctcaacaactacatctccctcttcaggaagaagaccaggaccgagaaggagaacaaggagctggagaacctggagatcaacctccgcaaggagatcgccaaggccttcaagggcaacgagggctacaagtccctcttcaagaaggacatcatcgagaccatcctcccagagttcctcgacgacaaggacgagatcgccctcgtcaactccttcaacggcttcaccaccgccttcaccggcttcttcgacaaccgcgagaatatgttctccgaggaagccaagtccacctccatcgccttcaggtgcatcaacgagaacctcacccgctacatcagcaacatggacatcttcgagaaggtcgacgccatcttcgacaagcacgaggtccaggagatcaaggagaagatcctgaacagcgactacgacgtcgaggacttcttcgagggcgagttcttcaacttcgtgctgacccaagagggcatcgacgtgtacaacgccatcatcggcggcttcgtgaccgagtccggcgagaagatcaagggcctcaacgagtacatcaacctctacaaccagaagaccaagcagaagctcccaaagttcaagccactgtacaagcaagtcctcagcgaccgcgagtccctgagcttctacggcgagggctacacctccgacgaggaagtgctggaggtgttcaggaacaccctcaacaagaactccgagatcttctccagcatcaagaagctggagaaactgttcaagaacttcgacgagtactccagcgccggcatcttcgtgaagaacggcccagccatctccaccatctccaaggacatcttcggcgagtggaacgtcatccgcgacaagtggaacgccgagtacgacgacatccacctgaagaagaaggccgtcgtgaccgagaagtacgaggacgaccgccgcaagagcttcaagaagatcggctccttcagcctggagcaactccaggagtacgccgacgccgacctctccgtcgtggagaagctcaaggagatcatcatccaaaaggtggacgagatctacaaggtgtacggcagctccgagaaactgttcgacgccgacttcgtgctggagaagtccctcaagaagaacgacgccgtggtcgccatcatgaaggacctgctcgactccgtcaagagcttcgagaactacatcaaggccttcttcggcgagggcaaggagaccaacagggacgagtccttctacggcgacttcgtcctcgcctacgacatcctcctgaaggtcgaccacatctacgacgccatcaggaactacgtgacccaaaagccatacagcaaggacaagttcaagctctacttccagaacccacaattcatgggcggctgggacaaggacaaggagaccgactacagggccaccatcctccgctacggcagcaagtactacctggccatcatggacaagaagtacgccaagtgcctccagaagatcgacaaggacgacgtgaacggcaactacgagaagatcaactacaagctgctcccaggcccaaacaagatgctcccaaaggtgttcttctccaagaagtggatggcctactacaacccaagcgaggacatccaaaagatctacaagaacggcaccttcaagaagggcgacatgttcaacctcaacgactgccacaagctcatcgacttcttcaaggactccatcagccgctacccaaagtggagcaacgcctacgacttcaacttctccgagaccgagaagtacaaggacatcgccggcttctacagggaggtcgaggagcaaggctacaaggtgtccttcgagtccgccagcaagaaggaagtggacaagctcgtggaggaaggcaagctgtacatgttccagatctacaacaaggacttctccgacaagagccacggcaccccaaacctccacaccatgtacttcaagctcctgttcgacgagaacaaccacggccaaatcaggctctccggcggcgccgagctgttcatgcgcagggccagcctcaagaaggaggagctcgtcgtgcacccagccaacagcccaatcgccaacaagaacccagacaacccaaagaagaccaccaccctctcctacgacgtgtacaaggacaagaggttcagcgaggaccaatacgagctgcacatcccaatcgccatcaacaagtgcccaaagaatatcttcaagatcaacaccgaggtgcgcgtcctgctcaagcacgacgacaacccatacgtcatcggcatcgacaggggcgagcgcaacctgctctacatcgtggtcgtggacggcaagggcaacatcgtggagcaatacagcctcaacgagatcatcaacaacttcaacggcatcaggatcaagaccgactaccactccctgctcgacaagaaggagaaggagaggttcgaggccaggcaaaactggacctccatcgagaacatcaaggagctgaaggccggctacatctcccaggtggtccacaagatctgcgagctcgtggagaagtacgacgccgtgatcgccctggaggacctcaacagcggcttcaagaacagccgcgtcaaggtggagaagcaagtctaccaaaagttcgagaagatgctgatcgacaagctgaactacatggtggacaagaagtccaacccatgcgccaccggcggcgccctcaagggctaccaaatcaccaacaagttcgagagcttcaagtccatgagcacccaaaacggcttcatcttctacatcccagcctggctcaccagcaagatcgacccatccaccggcttcgtcaacctgctcaagaccaagtacaccagcatcgccgacagcaagaagttcatctccagcttcgaccgcatcatgtatgtgccagaggaagacctcttcgagttcgccctggactacaagaacttctccaggaccgacgccgactacatcaagaagtggaagctctactcctacggcaacaggatccgcatcttcaggaacccaaagaagaacaacgtgttcgactgggaggaagtgtgcctgacctccgcctacaaggagctgttcaacaagtacggcatcaactaccagcaaggcgacatccgcgccctgctctgcgagcagagcgacaaggccttctacagctccttcatggccctgatgagcctcatgctgcaaatgcgcaactccatcaccggccgcaccgacgtggacttcctcatcagcccagtcaagaacagcgacggcatcttctacgactcccgcaactacgaggcccaagagaacgccatcctgccaaagaacgccgacgccaacggcgcctacaacatcgcccgcaaggtgctgtgggccatcggccaattcaagaaggccgaggacgagaagctggacaaggtgaagatcgccatcagcaacaaggagtggctggagtacgcccaaacctccgtgaagcacaagcgcccagccgccaccaagaaggcgggccaagccaagaagaagaagggcagctacccatacgacgtgccagactacgcctacccatacgacgtcccagactacgcctacccatacgacgtgccagactacgcctgaaacgcatccaatccaatatggacgattggacgtggaaggtttcctgtgtcttctacgatataataattggtcgctgtcctgtgtggatgtttcctgtgtcttctacgatatattaatgtacttctgtctgtttcgtttcgtatgtacaatcttccgggtatcgttcatgtatccctttgaataataacaaataaaatcgggtttgtcacggtaatctgaataaccttttattagccaaagtttaaccaaaattacgtattgcacgtaaaaattaagctagtttttggagagccggagacagacgtgatctatgtcgggtgcggagaaagaggtaatgaaatggcaggagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtctccaccatggctcagtcaaagcacgggctcacaaaggagatgactatgaagtacaggatggagggctgcgtcgatgggcacaagttcgttatcaccggcgaggggatcggctacccgttcaagggcaagcaggcgattaacctgtgcgtggtcgagggcgggccactgcccttcgctgaggatatcctctccgccgcgttcatgtacggcaacagggttttcaccgagtacccgcaggacattgtcgattacttcaagaattcctgcccagctgggtacacgtgggacaggagcttcctcttcgaggatggcgctgtgtgcatctgcaacgccgacattaccgtttcggtggaggagaattgcatgtaccacgagtctaagttctacggggtgaacttcccagctgacggccccgtcatgaagaagatgacggataattgggagccatcatgcgagaagatcattccagtgcctaagcaggggatcctgaagggcgacgtctccatgtacctcctgctcaaggatggcggcaggctcaggtgccagttcgacacagtctacaaggccaagagcgttccacgcaagatgcctgactggcacttcatccagcataagctgactcgggaggaccgctcggatgcgaagaaccagaagtggcacctgactgagcacgctatcgcctcggggtccgccctcccttgaaacgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgtgatctatgtcgggtgcggagaaagaggtaatgaaatggcagtgttaaacgaattcggccggccggtaccccttaggcctagggctagcggcgcgccacgcgtactagtcgatcgcctaggggtaccgttaacttaattaatctggaggtcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctagctgccggacgacgagctcctcccccctccccctccgccgccgccgcgccggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagacgatatctagaccaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgaaacgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgtggttaaacgaattcggccggccggtaccccttaggctcgagctaattaattaggccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcag

SEQ ID NO: 21 - DNA, 559 bp, Artificial
  TaNLD_4AS_8bp deletion mutant
acgatggtggcgatgacgcagatcaccaagaagatgatgggcaaggacagggaggagctgtacccggtgaagccgtcggactgcggcaagttcctggtgatgtccgtcggcaccggctcgacgtccgaccaggggctgtacacggcgaagcagtgctcccagtggggcatcatcagctggctccgcaacaagggcatggcgcccatcatcgacatcttcatggcagccagctccgacctcgtcgacatccacgccgccgtgctcttccagtcgctgcacagcgacggcgactacctccgcatccaggacaactcgctccacggcccggcggccacggtggacgctgccacgcccgagaacatggcggagctcctcaggatcggcgagcggatgctggcgcagagggtgtccagggtgaacgtcgaggtacgaggaggtaaagggggccgggagcaacgccgacgcgctcgccgggttcgccaggcagctctccgacgagaggaggacaaggctcgagcgccggcgtggtggcgccggccgtctgaaatccagacgctga

SEQ ID NO: 22 - protein, 188 aa, Artificial
  TaNLD_4AS_8bp deletion mutant exon 4
TMVAMTQITKKMMGKDREELYPVKPSDCGKFLVMSVGTGSTSDQGLYTAKQCSQWGIISWLRNKGMAPIIDIFMAASSDLVDIHAAVLFQSLHSDGDYLRIQDNSLHGPAATVDAATPENMAELLRIGERMLAQRVSRVNVEVRGGKGGREQRRRARRVRQAALRREEDKARAPAWWRRPSEIQTLTL

SEQ ID NO: 23 - DNA, 556 bp, Artificial
  TaNLD_4BL_11bp deletion mutant
acgatggtggcgatgacgcagatcaccaagaagatgatgggcaaggacagggaggagctgtacccggtggagccgtcggactgcggcaagttcctggtgctgtccgtcgggaccggctcgacgtccgaccaggggctgtacacggcgaagcagtgctcccagtggggcatcatcagctggctgcgcaacaagggcatggcgcccatcatcgacatcttcatggcggccagctccgacctcgtcgacatccacgccgccgtgctcttccagtcgctgcacagcgacgccaactacctccgcatccaggacaactcgctccacggcccggcggccacggtggacgccgccacgcccgagaacatggcggagctcctcaggatcggcgagcggatgctggcgcagagggtgtccagggtgaacgaggtacgaggagataaagggggccgggagcaacgccgacgcgctcgccggcttcgccaggcagctctccgacgagaggaggacaaggctcggccgccggcgcggtggcgccggccgcctgaaatccagccgctga

SEQ ID NO: 24 - protein, 187 aa, Artificial
  TaNLD_4BL_11bp deletion mutant exon 4
TMVAMTQITKKMMGKDREELYPVEPSDCGKFLVLSVGTGSTSDQGLYTAKQCSQWGIISWLRNKGMAPIIDIFMAASSDLVDIHAAVLFQSLHSDANYLRIQDNSLHGPAATVDAATPENMAELLRIGERMLAQRVSRVNEVRGDKGGREQRRRARRLRQAALRREEDKARPPARWRRPPEIQPLIL

SEQ ID NO: 25 - DNA, 541 bp, Artificial
  TaNLD_4BL_26bp deletion mutant
acgatggtggcgatgacgcagatcaccaagaagatgatgggcaaggacagggaggagctgtacccggtggagccgtcggactgcggcaagttcctggtgctgtccgtcgggaccggctcgacgtccgaccaggggctgtacacggcgaagcagtgctcccagtggggcatcatcagctggctgcgcaacaagggcatggcgcccatcatcgacatcttcatggcggccagctccgacctcgtcgacatccacgccgccgtgctcttccagtcgctgcacagcgacgccaactacctccgcatccaggacaactcgctccacggcccggcggccacggtggacgccgccacgcccgagaacatggcggagctcctcaggatcggcgagcggatgctggcgcagagggtggtacgaggagataaagggggccgggagcaacgccgacgcgctcgccggcttcgccaggcagctctccgacgagaggaggacaaggctcggccgccggcgcggtggcgccggccgcctgaaatccagccgctga

SEQ ID NO: 26 - protein, 182 aa, Artificial
  TaNLD_4BL_26bp deletion mutant exon 4
TMVAMTQITKKMMGKDREELYPVEPSDCGKFLVLSVGTGSTSDQGLYTAKQCSQWGIISWLRNKGMAPIIDIFMAASSDLVDIHAAVLFQSLHSDANYLRIQDNSLHGPAATVDAATPENMAELLRIGERMLAQRVVRGDKGGREQRRRARRLRQAALRREEDKARPPARWRRPPEIQPLIL

SEQ ID NO: 27 - DNA, 560 bp, Artificial
  TaNLD_4DL_7bp deletion mutant
acgatggtggcgatgacgcagatcaccaagaagatgatgggcaaggacagggaggagctgtacccggtggagccgtcggactgcggcaagttcctggtgctgtccgtcgggaccggctcgacgtccgaccaggggctgtacacggcgaagcagtgctcccagtggggcatcatcagctggctgcgcaacaagggcatggcgcccatcatcgacatcttcatggccgccagctccgacctcgtcgacatccacgccgccgtgctcttccagtcgctgcacagcgacgccaactacctccgcatccaggacaactcgctccacggcccggcggccacggtggacgccgccacgcccgagaacatggcggagctcctcaggatcggcgagcggatgctggcgcagagggtgtccagggtgaacgtcggaggtacgaggaggtaaagggggccgggaacaacgccgacgcgctcgccggcttcgccaggcagctctccgacgagaggaggacaaggctcgggagccggcgcggtggcgccggcctcctgaaatccagccgctga

SEQ ID NO: 28 - protein, 146 aa, Artificial
  TaNLD_4DL_7bp deletion mutant exon 4
TMVAMTQITKKMMGKDREELYPVEPSDCGKFLVLSVGTGSTSDQGLYTAKQCSQWGIISWLRNKGMAPIIDIFMAASSDLVDIHAAVLFQSLHSDANYLRIQDNSLHGPAATVDAATPENMAELLRIGERMLAQRVSRVNVGGTRR

SEQ ID NO: 29 - DNA, 559 bp, Artificial
  TaNLD_4DL_8bp deletion mutant
acgatggtggcgatgacgcagatcaccaagaagatgatgggcaaggacagggaggagctgtacccggtggagccgtcggactgcggcaagttcctggtgctgtccgtcgggaccggctcgacgtccgaccaggggctgtacacggcgaagcagtgctcccagtggggcatcatcagctggctgcgcaacaagggcatggcgcccatcatcgacatcttcatggccgccagctccgacctcgtcgacatccacgccgccgtgctcttccagtcgctgcacagcgacgccaactacctccgcatccaggacaactcgctccacggcccggcggccacggtggacgccgccacgcccgagaacatggcggagctcctcaggatcggcgagcggatgctggcgcagagggtgtccagggtgaacgtcgaggtacgaggaggtaaagggggccgggaacaacgccgacgcgctcgccggcttcgccaggcagctctccgacgagaggaggacaaggctcgggagccggcgcggtggcgccggcctcctgaaatccagccgctga

SEQ ID NO: 30 - protein, 188 aa, Artificial
  TaNLD_4DL_8bp deletion mutant exon 4
TMVAMTQITKKMMGKDREELYPVEPSDCGKFLVLSVGTGSTSDQGLYTAKQCSQWGIISWLRNKGMAPIIDIFMAASSDLVDIHAAVLFQSLHSDANYLRIQDNSLHGPAATVDAATPENMAELLRIGERMLAQRVSRVNVEVRGGKGGREQRRRARRLRQAALRREEDKAREPARWRRPPEIQPLIL

SEQ ID NO: 31 - DNA, 547 bp, Artificial
  TaNLD_4DL_20bp deletion mutant
acgatggtggcgatgacgcagatcaccaagaagatgatgggcaaggacagggaggagctgtacccggtggagccgtcggactgcggcaagttcctggtgctgtccgtcgggaccggctcgacgtccgaccaggggctgtacacggcgaagcagtgctcccagtggggcatcatcagctggctgcgcaacaagggcatggcgcccatcatcgacatcttcatggccgccagctccgacctcgtcgacatccacgccgccgtgctcttccagtcgctgcacagcgacgccaactacctccgcatccaggacaactcgctccacggcccggcggccacggtggacgccgccacgcccgagaacatggcggagctcctcaggatcggcgagcggatgctggcgcagagggtgtccagggtgaacgtcggtaaagggggccgggaacaacgccgacgcgctcgccggcttcgccaggcagctctccgacgagaggaggacaaggctcgggagccggcgcggtggcgccggcctcctgaaatccagccgctga

SEQ ID NO: 32 - protein, 184 aa, Artificial
  TaNLD_4DL_20bp deletion mutant exon 4
TMVAMTQITKKMMGKDREELYPVEPSDCGKFLVLSVGTGSTSDQGLYTAKQCSQWGIISWLRNKGMAPIIDIFMAASSDLVDIHAAVLFQSLHSDANYLRIQDNSLHGPAATVDAATPENMAELLRIGERMLAQRVSRVNVGKGGREQRRRARRLRQAALRREEDKAREPARWRRPPEIQPLIL

SEQ ID NO: 33 - protein, 399 aa, Zea mays
MASYSSRRPCNTCSTKAMAGSVVGEPVVLGQRVTVLTVDGGGVRGLIPGTILAFLEARLQELDGPEARLADYFDYIAGTSTGGLITAMLTAPGKDKRPLYAAKDINYFYMENCPRIFPQKSRLAAAMSALRKPKYNGKCMRSLIRSILGETRVSETLTNVIIPAFDIRLLQPIIFSTYDAKSTPLKNALLSDVCIGTSAAPTYLPAHYFQTEDANGKEREYNLIDGGVAANNPTMVAMTQITKKMLASKDKAEELYPVNPSNCRRFLVLSIGTGSTSEQGLYTARQCSRWGICRWLRNNGMAPIIDIFMAASSDLVDIHVAAMFQSLHSDGDYLRIQDNSLRGAAATVDAATPENMRTLVGIGERMLAQRVSRVNVETGSEVRTGDRRRKQCRCPRWAR

SEQ ID NO: 34 - DNA, 566 bp, Triticum aestivum
acgatggtggcgatgacgcagatcaccaagaagatgatgggcaaggacagggaggagctgtacccggtggagccgtcggactgcggcaagttcctggtgctgtccgtcgggaccggctcgacgtccgaccaggggctgtacacggcgaagcagtgctcccagtggggcatcatcagctggctgcgcaacaagggcatggcgcccatcatcgacatcttcatggccgccagctccgacctcgtcgacatccacgccgccgtgctcttccagtcgctgcacagcgacgccaactacctccgcatccaggacaactcgctccacggcccggcggccacggtggacgccgccacgcccgagaacatggcggagctcctcaggatcggcgagcggatgctggcgcagagggtgtccagggtgaacgtcgagacgggaggtacgaggaggtaaagggggccgggaacaacgccgacgcgctcgccggcttcgccaggcagctctccgacgagaggaggacaaggctcgggagccggcgcggtggcgccggcctcctgaaatccagccgctga

SEQ ID NO: 35 - DNA, 566 bp, Triticum aestivum
acgatggtggcgatgacgcagatcaccaagaagatgatgggcaaggacagggaggagctgtacccggtggagccgtcggactgcggcaagttcctggtgctgtccgtcgggaccggctcgacgtccgaccaggggctgtacacggcgaagcagtgctcccagtggggcatcatcagctggctgcgcaacaagggcatggcgcccatcatcgacatcttcatggccgccagctccgacctcgtcgacatccacgccgccgtgctcttccagtcgctgcacagcgacgccaactacctccgcatccaggacaactcgctccacggcccggcggccacggtggacgccgccacgcccgagaacatggcggagctcctcaggatcggcgagcggatgctggcgcagagggtgtccagggtgaacgtcgagaccggaggtacgaggaggtaaagggggccgggaacaacgccgacgcgctcgccggcttcgccaggcagctctccgacgagaggaggacaaggctcgggagccggcgcggtggcgccggcctcctgaaatccagccgctga

SEQ ID NO: 36 - DNA, 562 bp, Triticum aestivum
acgatggtggcgatgacgcagatcaccaagaagatgatgggcaaggacagggaggagctgtacccggtggagccgtcggactgcggcaagttcctggtgctgtccgtcgggaccggctcgacgtccgaccaggggctgtacacggcgaagcagtgctcccagtggggcatcatcagctggctgcgcaacaagggcatggcgcccatcatcgacatcttcatggccgccagctccgacctcgtcgacatccacgccgccgtgctcttccagtcgctgcacagcgacgccaactacctccgcatccaggacaactcgctccacggcccggcggccacggtggacgccgccacgcccgagaacatggcggagctcctcaggatcggcgagcggatgctggcgcagagggtgtccagggtgaacgtcgaggaggtacgaggaggtaaagggggccgggaacaacgccgacgcgctcgccggcttcgccaggcagctctccgacgagaggaggacaaggctcgggagccggcgcggtggcgccggcctcctgaaatccagccgctga

SEQ ID NO: 37 - DNA, 562 bp, Triticum aestivum
acgatggtggcgatgacgcagatcaccaagaagatgatgggcaaggacagggaggagctgtacccggtggagccgtcggactgcggcaagttcctggtgctgtccgtcgggaccggctcgacgtccgaccaggggctgtacacggcgaagcagtgctcccagtggggcatcatcagctggctgcgcaacaagggcatggcgcccatcatcgacatcttcatggccgccagctccgacctcgtcgacatccacgccgccgtgctcttccagtcgctgcacagcgacgccaactacctccgcatccaggacaactcgctccacggcccggcggccacggtggacgccgccacgcccgagaacatggcggagctcctcaggatcggcgagcggatgctggcgcagagggtgtccagggtgaacgtcggggaggtacgaggaggtaaagggggccgggaacaacgccgacgcgctcgccggcttcgccaggcagctctccgacgagaggaggacaaggctcgggagccggcgcggtggcgccggcctcctgaaatccagccgctga

SEQ ID NO: 38 - DNA, 557 bp, Triticum aestivum
acgatggtggcgatgacgcagatcaccaagaagatgatgggcaaggacagggaggagctgtacccggtggagccgtcggactgcggcaagttcctggtgctgtccgtcgggaccggctcgacgtccgaccaggggctgtacacggcgaagcagtgctcccagtggggcatcatcagctggctgcgcaacaagggcatggcgcccatcatcgacatcttcatggccgccagctccgacctcgtcgacatccacgccgccgtgctcttccagtcgctgcacagcgacgccaactacctccgcatccaggacaactcgctccacggcccggcggccacggtggacgccgccacgcccgagaacatggcggagctcctcaggatcggcgagcggatgctggcgcagagggtgtccagggtgaacggaggtacgaggaggtaaagggggccgggaacaacgccgacgcgctcgccggcttcgccaggcagctctccgacgagaggaggacaaggctcgggagccggcgcggtggcgccggcctcctgaaatccagccgctga

SEQ ID NO: 39 - DNA, 556 bp, Triticum aestivum
acgatggtggcgatgacgcagatcaccaagaagatgatgggcaaggacagggaggagctgtacccggtggagccgtcggactgcggcaagttcctggtgctgtccgtcgggaccggctcgacgtccgaccaggggctgtacacggcgaagcagtgctcccagtggggcatcatcagctggctgcgcaacaagggcatggcgcccatcatcgacatcttcatggccgccagctccgacctcgtcgacatccacgccgccgtgctcttccagtcgctgcacagcgacgccaactacctccgcatccaggacaactcgctccacggcccggcggccacggtggacgccgccacgcccgagaacatggcggagctcctcaggatcggcgagcggatgctggcgcagagggtgtccagggtgaacgtggtacgaggaggtaaagggggccgggaacaacgccgacgcgctcgccggcttcgccaggcagctctccgacgagaggaggacaaggctcgggagccggcgcggtggcgccggcctcctgaaatccagccgctga

SEQ ID NO: 40 - DNA, 555 bp, Triticum aestivum
acgatggtggcgatgacgcagatcaccaagaagatgatgggcaaggacagggaggagctgtacccggtggagccgtcggactgcggcaagttcctggtgctgtccgtcgggaccggctcgacgtccgaccaggggctgtacacggcgaagcagtgctcccagtggggcatcatcagctggctgcgcaacaagggcatggcgcccatcatcgacatcttcatggccgccagctccgacctcgtcgacatccacgccgccgtgctcttccagtcgctgcacagcgacgccaactacctccgcatccaggacaactcgctccacggcccggcggccacggtggacgccgccacgcccgagaacatggcggagctcctcaggatcggcgagcggatgctggcgcagagggtgtccagggtgaacgtctacgaggaggtaaagggggccgggaacaacgccgacgcgctcgccggcttcgccaggcagctctccgacgagaggaggacaaggctcgggagccggcgcggtggcgccggcctcctgaaatccagccgctga

SEQ ID NO: 41 - protein, 148 aa, Triticum aestivum
TMVAMTQITKKMMGKDREELYPVEPSDCGKFLVLSVGTGSTSDQGLYTAKQCSQWGIISWLRNKGMAPIIDIFMAASSDLVDIHAAVLFQSLHSDANYLRIQDNSLHGPAATVDAATPENMAELLRIGERMLAQRVSRVNVETGGTRR

SEQ ID NO: 42 - protein, 148 aa, Triticum aestivum; identical to SEQ ID NO: 41

SEQ ID NO: 43 - protein, 189 aa, Triticum aestivum
TMVAMTQITKKMMGKDREELYPVEPSDCGKFLVLSVGTGSTSDQGLYTAKQCSQWGIISWLRNKGMAPIIDIFMAASSDLVDIHAAVLFQSLHSDANYLRIQDNSLHGPAATVDAATPENMAELLRIGERMLAQRVSRVNVEEVRGGKGGREQRRRARRLRQAALRREEDKAREPARWRRPPEIQPLIL

SEQ ID NO: 44 - protein, 189 aa, Triticum aestivum
TMVAMTQITKKMMGKDREELYPVEPSDCGKFLVLSVGTGSTSDQGLYTAKQCSQWGIISWLRNKGMAPIIDIFMAASSDLVDIHAAVLFQSLHSDANYLRIQDNSLHGPAATVDAATPENMAELLRIGERMLAQRVSRVNVGEVRGGKGGREQRRRARRLRQAALRREEDKAREPARWRRPPEIQPLIL

SEQ ID NO: 45 - protein, 145 aa, Triticum aestivum
TMVAMTQITKKMMGKDREELYPVEPSDCGKFLVLSVGTGSTSDQGLYTAKQCSQWGIISWLRNKGMAPIIDIFMAASSDLVDIHAAVLFQSLHSDANYLRIQDNSLHGPAATVDAATPENMAELLRIGERMLAQRVSRVNGGTRR

SEQ ID NO: 46 - protein, 187 aa, Triticum aestivum
TMVAMTQITKKMMGKDREELYPVEPSDCGKFLVLSVGTGSTSDQGLYTAKQCSQWGIISWLRNKGMAPIIDIFMAASSDLVDIHAAVLFQSLHSDANYLRIQDNSLHGPAATVDAATPENMAELLRIGERMLAQRVSRVNVVRGGKGGREQRRRARRLRQAALRREEDKAREPARWRRPPEIQPLIL

SEQ ID NO: 47 - protein, 184 aa, Triticum aestivum
TMVAMTQITKKMMGKDREELYPVEPSDCGKFLVLSVGTGSTSDQGLYTAKQCSQWGIISWLRNKGMAPIIDIFMAASSDLVDIHAAVLFQSLHSDANYLRIQDNSLHGPAATVDAATPENMAELLRIGERMLAQRVSRVNVYEEVKGAGNNADALAGFARQLSDERRTRLGSRRGGAGLLKSSR

SEQ ID NO: 48 - protein, 1401 aa, Artificial [sequence omitted]

SEQ ID NO: 49 - DNA, 13453 bp, Artificial
  Construct pBIOS11489 T-DNA region
aaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctaattaattaggctagaggataactataacggtcctaaggtagcgatacgcatcgtatcggagtctcagcttacgcttgtagactgatccgtctcacgagtctagacgttctaggtcactgacgtcagtctgttggaaggcacgatcgctgagtgtctaatccaattctgctggctacagacctacgtgcaactggacttgttgacgtctcgttgagtgcacctatcgacaggcactagcgacgcacgacgtaacctgatacgaggcttaattaattagggagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtctccaccatggctcctaagaagaagcgcaaggttgggatccacggggttccggcggctatggacaagaagtacagcatcgggctcgacatcgggacgaacagcgttgggtgggcggtcatcacggacgagtacaaggttccctccaagaagttcaaggtgctggggaacaccgaccgccactccatcaagaagaacctcatcggggccctcctgttcgacagcggggagaccgctgaggctacgaggctcaagagaaccgcgaggcgcagatacacgagaaggaagaacaggatctgctacctccaagagatcttctccaacgagatggccaaggttgacgattcattcttccaccgcctggaggagtctttcctcgtggaggaggacaagaagcacgagcggcatcccatcttcgggaacatcgtcgacgaggttgcctaccacgagaagtaccctacgatctaccatctgcggaagaagctcgtggacagcaccgataaggcggacctcagactgatctacctcgccctggcgcacatgatcaagttccgcgggcatttcctgatcgagggggatctcaacccagacaactccgatgttgacaagctgttcatccaactcgtgcagacctacaaccaactcttcgaggagaacccgatcaacgcttcgggggtcgacgctaaggccatcctgtcagcccgcctctcgaagtcacgcaggctggagaacctgatcgcgcagctcccaggggagaagaagaacgggctgttcgggaacctcatcgctctctctctggggctcacgccgaacttcaagtccaacttcgatctcgccgaggacgcgaagctgcaactcagcaaggacacctacgacgatgacctcgataacctcctggcgcagatcggggatcaatacgctgacctgttcctcgccgcgaagaacctgtcggacgccatcctcctgtcagatatcctccgcgtgaacaccgagatcacgaaggccccactctctgcgtccatgatcaagcgctacgacgagcaccatcaggatctgaccctcctgaaggcgctggtccgccaacagctcccggagaagtacaaggagatcttcttcgatcagtcgaagaacgggtacgctgggtacatcgacgggggggcgtcacaagaggagttctacaagttcatcaagccaatcctggagaagatggacgggacggaggagctcctggtgaagctcaacagggaggacctcctgcggaagcagagaaccttcgataacgggtccatcccccaccaaatccatctcggggagctgcacgccatcctgagaaggcaagaggacttctaccctttcctcaaggataaccgggagaagatcgagaagatcctgaccttcagaatcccatactacgtcgggccgctcgctcgggggaacagcagattcgcctggatgacccgcaagtcggaggagaccatcacgccgtggaacttcgaggaggtggtcgacaagggggcgagcgctcagtcgttcatcgagaggatgaccaacttcgacaagaacctgcccaacgagaaggtgctccctaagcactcgctcctgtacgagtacttcaccgtctacaacgagctcacgaaggtgaagtatgtgaccgaggggatgagaaagcctgccttcctgtccggggagcagaagaaggcgatcgtggacctcctgttcaagaccaaccggaaggtcacggttaagcaactcaaggaggactacttcaagaagatcgagtgcttcgattcggtggagatctcaggggtcgaggacaggttcaacgcctccctcgggacctaccacgatctcctgaagatcatcaaggataaggacttcctggacaacgaggagaacgaggacatcctggaggacatcgtgctgaccctcacgctgttcgaggacagggagatgatcgaggagcgcctgaagacgtacgcccatctcttcgatgacaaggtcatgaagcaactcaagagaagaagatacaccgggtgggggaggctgtcccgcaagctcatcaacgggatccgggacaagcagtcagggaagaccatcctcgacttcctgaagtctgatgggttcgccaacaggaacttcatgcaactgatccacgatgactcactcaccttcaaggaggatatccaaaaggctcaggtgagcgggcagggggactcgctgcacgagcatatcgctaacctcgctgggtcccctgcgatcaagaaggggatcctgcagaccgtgaaggttgtggacgagctcgtgaaggtcatggggcggcataagcctgagaacatcgtcatcgagatggccagagagaaccaaaccacgcagaaggggcaaaagaactctagggagcgcatgaagcgcatcgaggaggggatcaaggagctggggtcccaaatcctcaaggagcacccagttgagaacacccaactgcagaacgagaagctctacctgtactacctccagaacgggagggatatgtatgtggaccaagagctggatatcaaccgcctcagcgattacgacgttgatcatatcgtgccccagtctttcctgaaggatgactccatcgacaacaaggtcctcaccaggtcggacaagaaccgcgggaagtcagataacgttccatctgaggaggtcgttaagaagatgaagaactactggaggcagctcctgaacgccaagctgatcacgcaaaggaagttcgacaacctcaccaaggctgagagaggggggctctcagagctggacaaggctgggttcatcaagcggcagctggtcgagaccagacaaatcacgaagcacgttgcgcaaatcctcgactctcggatgaacacgaagtacgatgagaacgacaagctgatcagggaggtgaaggtcatcaccctgaagtctaagctcgtctccgacttccgcaaggacttccagttctacaaggttcgcgagatcaacaactaccaccatgcgcatgacgcttacctcaacgctgtggtcgggaccgccctgatcaagaagtacccgaagctggagagcgagttcgtgtacggggactacaaggtttacgatgtgcgcaagatgatcgccaagtcggagcaagagatcgggaaggccaccgccaagtacttcttctactcaaacatcatgaacttctttaagaccgagatcacgctggccaacggggagatccggaagagacccctcatcgagaccaacggggagacgggggagatcgtgtgggacaaggggagggatttcgcgaccgtccgcaaggttctctccatgccccaggtgaacatcgtcaagaagaccgaggtccaaacgggggggttctcaaaggagtctatcctgcctaagcggaacagcgacaagctcatcgccagaaagaaggactgggatccaaagaagtacggggggttcgacagccctaccgtcgcgtactcggttctggttgtggctaaggttgagaaggggaagtccaagaagctcaagagcgtgaaggagctcctggggatcaccatcatggagcgctccagcttcgagaagaacccaatcgacttcctggaggccaaggggtacaaggaggtgaagaaggacctgatcatcaagctcccgaagtactctctcttcgagctggagaacgggaggaagcgcatgctggcttccgctggggagctccaaaaggggaacgagctcgccctgccatccaagtatgtgaacttcctctacctggcgtcccactacgagaagctcaaggggagcccggaggacaacgagcaaaagcagctgttcgtcgagcagcacaagcattacctcgacgagatcatcgagcaaatctccgagttctccaagcgcgtgatcctcgcggacgctaacctggataaggtcctctccgcttacaacaagcaccgggacaagcccatcagagagcaggccgagaacatcatccatctcttcaccctgacgaacctcggggcccctgctgccttcaagtacttcgacaccacgatcgatcggaagagatacaccagcacgaaggaggtcctggacgcgaccctcatccaccaatctatcacggggctgtacgagacgagaatcgacctgtcacaactggggggggacaagaggccggcggcaaccaagaaggcgggccaggccaagaagaagaagtgaaacgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgtgctctcttaaggtagcggagaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttggcgaagcagtgctcccagtgttttagagctagaaatagcaagttaaaataaggctagtccgttatcaacttgaaaaagtggcaccgagtcggtgctttttttaaggaatctttaaacatacgaacagatcacttaaagttcttctgaagcaacttaaagttatcaggcatgcatggatcttggaggaatcagatgtgcagtcagggaccatagcacaagacaggcgtcttctactggtgctaccagcaaatgctggaagccgggaacactgggtacgttggaaaccacgtgatgtgaagaagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctgatttaaaagagttgtgcagatgatccgtggcaggacaactcgctccacggccgttttagagctagaaatagcaagttaaaataaggctagtccgttatcaacttgaaaaagtggcaccgagtcggtgctttttttgtgtggcaaacagctattatgggtattatgggtggagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtctccaccatggctcagtcaaagcacgggctcacaaaggagatgactatgaagtacaggatggagggctgcgtcgatgggcacaagttcgttatcaccggcgaggggatcggctacccgttcaagggcaagcaggcgattaacctgtgcgtggtcgagggcgggccactgcccttcgctgaggatatcctctccgccgcgttcatgtacggcaacagggttttcaccgagtacccgcaggacattgtcgattacttcaagaattcctgcccagctgggtacacgtgggacaggagcttcctcttcgaggatggcgctgtgtgcatctgcaacgccgacattaccgtttcggtggaggagaattgcatgtaccacgagtctaagttctacggggtgaacttcccagctgacggccccgtcatgaagaagatgacggataattgggagccatcatgcgagaagatcattccagtgcctaagcaggggatcctgaagggcgacgtctccatgtacctcctgctcaaggatggcggcaggctcaggtgccagttcgacacagtctacaaggccaagagcgttccacgcaagatgcctgactggcacttcatccagcataagctgactcgggaggaccgctcggatgcgaagaaccagaagtggcacctgactgagcacgctatcgcctcggggtccgccctcccttgaaacgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgtgatctatgtcgggtgcggagaaagaggtaatgaaatggcagtgttaaacgaattcggccggccggtaccccttaggcctagggctagcggcgcgccacgcgtactagtcgatcgcctaggggtaccgttaacttaattaatctggaggtcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctagctgccggacgacgagctcctcccccctccccctccgccgccgccgcgccggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagacgatatctagaccaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgaaacgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgtggttaaacgaattcggccggccggtaccccttaggctcgagctaattaattaggccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcag

SEQ ID NO: 50 - DNA, 568 bp, Triticum aestivum
acgatggtggcgatgacgcagatcaccaagaagatgatgggcaaggacagggaggagctgtacccggtgaagccgtcggactgcggcaagttcctggtgatgtccgtcggcaccggctcgacgtccgaccaggggctgtacacggcgaagcagtgctcccaagtggggcatcatcagctggctccgcaacaagggcatggcgcccatcatcgacatcttcatggcagccagctccgacctcgtcgacatccacgccgccgtgctcttccagtcgctgcacagcgacggcgactacctccgcatccaggacaactcgctccacggcccggcggccacggtggacgctgccacgcccgagaacatggcggagctcctcaggatcggcgagcggatgctggcgcagagggtgtccagggtgaacgtcgagaccgggaggtacgaggaggtaaagggggccgggagcaacgccgacgcgctcgccgggttcgccaggcagctctccgacgagaggaggacaaggctcgagcgccggcgtggtggcgccggccgtctgaaatccagacgctga

SEQ ID NO: 51 - DNA, 568 bp, Triticum aestivum
acgatggtggcgatgacgcagatcaccaagaagatgatgggcaaggacagggaggagctgtacccggtggagccgtcggactgcggcaagttcctggtgctgtccgtcgggaccggctcgacgtccgaccaggggctgtacacggcgaagcagtgctcccaagtggggcatcatcagctggctgcgcaacaagggcatggcgcccatcatcgacatcttcatggcggccagctccgacctcgtcgacatccacgccgccgtgctcttccagtcgctgcacagcgacgccaactacctccgcatccaggacaactcgctccacggcccggcggccacggtggacgccgccacgcccgagaacatggcggagctcctcaggatcggcgagcggatgctggcgcagagggtgtccagggtgaacgtggagaccgggaggtacgaggagataaagggggccgggagcaacgccgacgcgctcgccggcttcgccaggcagctctccgacgagaggaggacaaggctcggccgccggcgcggtggcgccggccgcctgaaatccagccgctga

SEQ ID NO: 52 - DNA, 568 bp, Triticum aestivum
acgatggtggcgatgacgcagatcaccaagaagatgatgggcaaggacagggaggagctgtacccggtggagccgtcggactgcggcaagttcctggtgctgtccgtcgggaccggctcgacgtccgaccaggggctgtacacggcgaagcagtgctccctagtggggcatcatcagctggctgcgcaacaagggcatggcgcccatcatcgacatcttcatggccgccagctccgacctcgtcgacatccacgccgccgtgctcttccagtcgctgcacagcgacgccaactacctccgcatccaggacaactcgctccacggcccggcggccacggtggacgccgccacgcccgagaacatggcggagctcctcaggatcggcgagcggatgctggcgcagagggtgtccagggtgaacgtcgagaccgggaggtacgaggaggtaaagggggccgggaacaacgccgacgcgctcgccggcttcgccaggcagctctccgacgagaggaggacaaggctcgggagccggcgcggtggcgccggcctcctgaaatccagccgctga

SEQ ID NO: 53 - DNA, 567 bp, Triticum aestivum
acgatggtggcgatgacgcagatcaccaagaagatgatgggcaaggacagggaggagctgtacccggtggagccgtcggactgcggcaagttcctggtgctgtccgtcgggaccggctcgacgtccgaccaggggctgtacacggcgaagcagtgctccaagtggggcatcatcagctggctgcgcaacaagggcatggcgcccatcatcgacatcttcatggccgccagctccgacctcgtcgacatccacgccgccgtgctcttccagtcgctgcacagcgacgccaactacctccgcatccaggacaactcgctccacggcccggcggccacggtggacgccgccacgcccgagaacatggcggagctcctcaggatcggcgagcggatgctggcgcagagggtgtccagggtgaacgtcgagaccgggaggtacgaggaggtaaagggggccgggaacaacgccgacgcgctcgccggcttcgccaggcagctctccgacgagaggaggacaaggctcgggagccggcgcggtggcgccggcctcctgaaatccagccgctga

SEQ ID NO: 54 - DNA, 566 bp, Triticum aestivum
acgatggtggcgatgacgcagatcaccaagaagatgatgggcaaggacagggaggagctgtacccggtgaagccgtcggactgcggcaagttcctggtgatgtccgtcggcaccggctcgacgtccgaccaggggctgtacacggcgaagcagtgctccagtggggcatcatcagctggctccgcaacaagggcatggcgcccatcatcgacatcttcatggcagccagctccgacctcgtcgacatccacgccgccgtgctcttccagtcgctgcacagcgacggcgactacctccgcatccaggacaactcgctccacggcccggcggccacggtggacgctgccacgcccgagaacatggcggagctcctcaggatcggcgagcggatgctggcgcagagggtgtccagggtgaacgtcgagaccgggaggtacgaggaggtaaagggggccgggagcaacgccgacgcgctcgccgggttcgccaggcagctctccgacgagaggaggacaaggctcgagcgccggcgtggtggcgccggccgtctgaaatccagacgctga

SEQ ID NO: 55 - DNA, 563 bp, Triticum aestivum
acgatggtggcgatgacgcagatcaccaagaagatgatgggcaaggacagggaggagctgtacccggtgaagccgtcggactgcggcaagttcctggtgatgtccgtcggcaccggctcgacgtccgaccaggggctgtacacggcgaagcagtgcagtggggcatcatcagctggctccgcaacaagggcatggcgcccatcatcgacatcttcatggcagccagctccgacctcgtcgacatccacgccgccgtgctcttccagtcgctgcacagcgacggcgactacctccgcatccaggacaactcgctccacggcccggcggccacggtggacgctgccacgcccgagaacatggcggagctcctcaggatcggcgagcggatgctggcgcagagggtgtccagggtgaacgtcgagaccgggaggtacgaggaggtaaagggggccgggagcaacgccgacgcgctcgccgggttcgccaggcagctctccgacgagaggaggacaaggctcgagcgccggcgtggtggcgccggccgtctgaaatccagacgctga

SEQ ID NO: 56 - DNA, 563 bp, Triticum aestivum
acgatggtggcgatgacgcagatcaccaagaagatgatgggcaaggacagggaggagctgtacccggtggagccgtcggactgcggcaagttcctggtgctgtccgtcgggaccggctcgacgtccgaccaggggctgtacacggcgaagcagtgcagtggggcatcatcagctggctgcgcaacaagggcatggcgcccatcatcgacatcttcatggcggccagctccgacctcgtcgacatccacgccgccgtgctcttccagtcgctgcacagcgacgccaactacctccgcatccaggacaactcgctccacggcccggcggccacggtggacgccgccacgcccgagaacatggcggagctcctcaggatcggcgagcggatgctggcgcagagggtgtccagggtgaacgtggagaccgggaggtacgaggagataaagggggccgggagcaacgccgacgcgctcgccggcttcgccaggcagctctccgacgagaggaggacaaggctcggccgccggcgcggtggcgccggccgcctgaaatccagccgctga

SEQ ID NO: 57 - DNA, 563 bp, Triticum aestivum
acgatggtggcgatgacgcagatcaccaagaagatgatgggcaaggacagggaggagctgtacccggtggagccgtcggactgcggcaagttcctggtgctgtccgtcgggaccggctcgacgtccgaccaggggctgtacacggcgaagcagtgcagtggggcatcatcagctggctgcgcaacaagggcatggcgcccatcatcgacatcttcatggccgccagctccgacctcgtcgacatccacgccgccgtgctcttccagtcgctgcacagcgacgccaactacctccgcatccaggacaactcgctccacggcccggcggccacggtggacgccgccacgcccgagaacatggcggagctcctcaggatcggcgagcggatgctggcgcagagggtgtccagggtgaacgtcgagaccgggaggtacgaggaggtaaagggggccgggaacaacgccgacgcgctcgccggcttcgccaggcagctctccgacgagaggaggacaaggctcgggagccggcgcggtggcgccggcctcctgaaatccagccgctga

SEQ ID NO: 58 - protein, 189 aa, Triticum aestivum
TMVAMTQITKKMMGKDREELYPVKPSDCGKFLVMSVGTGSTSDQGLYTAKQCSQVGHHQLAPQQGHGAHHRHLHGSQLRPRRHPRRRALPVAAQRRRLPPHPGQLAPRPGGHGGRCHAREHGGAPQDRRADAGAEGVQGERRDREVRGGKGGREQRRRARRVRQAALRREEDKARAPAWWRRPSEIQTL

SEQ ID NO: 59 - protein, 189 aa, Triticum aestivum
TMVAMTQITKKMMGKDREELYPVEPSDCGKFLVLSVGTGSTSDQGLYTAKQCSQVGHHQLAAQQGHGAHHRHLHGGQLRPRRHPRRRALPVAAQRRQLPPHPGQLAPRPGGHGGRRHAREHGGAPQDRRADAGAEGVQGERGDREVRGDKGGREQRRRARRLRQAALRREEDKARPPARWRRPPEIQPL

SEQ ID NO: 60 - protein, 189 aa, Triticum aestivum
TMVAMTQITKKMMGKDREELYPVEPSDCGKFLVLSVGTGSTSDQGLYTAKQCSLVGHHQLAAQQGHGAHHRHLHGRQLRPRRHPRRRALPVAAQRRQLPPHPGQLAPRPGGHGGRRHAREHGGAPQDRRADAGAEGVQGERRDREVRGGKGGREQRRRARRLRQAALRREEDKAREPARWRRPPEIQPL

SEQ ID NO: 61 - protein, 188 aa, Triticum aestivum
TMVAMTQITKKMMGKDREELYPVEPSDCGKFLVLSVGTGSTSDQGLYTAKQCSKWGIISWLRNKGMAPIIDIFMAASSDLVDIHAAVLFQSLHSDANYLRIQDNSLHGPAATVDAATPENMAELLRIGERMLAQRVSRVNVETGRYEEVKGAGNNADALAGFARQLSDERRTRLGSRRGGAGLLKSSR

SEQ ID NO: 62 - protein, 138 aa, Triticum aestivum
TMVAMTQITKKMMGKDREELYPVKPSDCGKFLVMSVGTGSTSDQGLYTAKQCSSGASSAGSATRAWRPSSTSSWQPAPTSSTSTPPCSSSRCTATATTSASRTTRSTARRPRWTLPRPRTWRSSSGSASGCWRRGCPG

SEQ ID NO: 63 - protein, 137 aa, Triticum aestivum
TMVAMTQITKKMMGKDREELYPVKPSDCGKFLVMSVGTGSTSDQGLYTAKQCSGASSAGSATRAWRPSSTSSWQPAPTSSTSTPPCSSSRCTATATTSASRTTRSTARRPRWTLPRPRTWRSSSGSASGCWRRGCPG

SEQ ID NO: 64 - protein, 137 aa, Triticum aestivum
TMVAMTQITKKMMGKDREELYPVEPSDCGKFLVLSVGTGSTSDQGLYTAKQCSGASSAGCATRAWRPSSTSSWRPAPTSSTSTPPCSSSRCTATPTTSASRTTRSTARRPRWTPPRPRTWRSSSGSASGCWRRGCPG

SEQ ID NO: 65 - protein, 137 aa, Triticum aestivum
TMVAMTQITKKMMGKDREELYPVEPSDCGKFLVLSVGTGSTSDQGLYTAKQCSGASSAGCATRAWRPSSTSSWPPAPTSSTSTPPCSSSRCTATPTTSASRTTRSTARRPRWTPPRPRTWRSSSGSASGCWRRGCPG

SEQ ID NO: 66 - DNA, 27 bp, Artificial
  TaNLD_4AS forward primer
gacttcacttacgcttcgtcatgagcg

SEQ ID NO: 67 - DNA, 22 bp, Artificial
  TaNLD_4AS reverse primer
gcttgccgaaataggtaggagg

SEQ ID NO: 68 - DNA, 29 bp, Artificial
  TaNLD_4BL /4DL forward primer
gaattaagatctgcctcctaccacagtcg

SEQ ID NO: 69 - DNA, 23 bp, Artificial
  TaNLD_4BL/4DL reverse primer
gaagctttctctacctatcccag

SEQ ID NO: 70 - DNA, 26 bp, Artificial
  TaNLD_4AS forward primer
cttctccacatacgacgtatatatgc

SEQ ID NO: 71 - DNA, 22 bp, Artificial
  TaNLD_4AS reverse primer
gctagccaaagtagggatgctg

SEQ ID NO: 72 - DNA, 25 bp, Artificial
  TaNLD_4BL forward primer
cgacgtatgctaattttatacgagg

SEQ ID NO: 73 - DNA, 22 bp, Artificial
  TaNLD_4BL reverse primer
gctagccaaagtagggatgctg

SEQ ID NO: 74 - DNA, 28 bp, Artificial
  TaNLD_4DL forward primer
cgacgtatgccaattttatatgtataag

SEQ ID NO: 75 - DNA, 23 bp, Artificial
  TaNLD_4DL reverse primer
gatgatcgtttaaccgatgttgg

SEQ ID NO: 76 - DNA, 19 bp, Artificial
  NLD crRNA amplicon forward primer
atccaggacaactcgctcc

SEQ ID NO: 77 - DNA, 20 bp, Artificial
  NLD crRNA amplicon reverse primer
agccttgtcctcctctcgtc

SEQ ID NO: 78 - DNA, 18 bp, Artificial
  NLD gRNA7 amplicon forward primer
gactgcggcaagttcctg

SEQ ID NO: 79 - DNA, 18 bp, Artificial
  NLD gRNA7 amplicon reverse primer
caccctggacaccctctg

SEQ ID NO: 80 - DNA, 23 bp, Artificial
  Guide NLD 4AS and 4DL genes (Cpf1)
cctcctcgtacctcccggtctcg

SEQ ID NO: 81 - DNA, 23 bp, Artificial
  Guide NLD 4BL gene (Cpf1)
tctcctcgtacctcccggtctcc

SEQ ID NO: 82 - DNA, 20 bp, Artificial
  Guide TaNLD-4AS, TaNLD-4BL and TaNLD-4DL (Cas9)
ggcgaagcagtgctcccagt

SEQ ID NO: 83 - protein, 420 aa, Artificial Sequence
  Consensus sequence
MASYWRRPCESCSTRAMAGSVVGQPVAPGQRVTVLTIDGGGIRGIIPGTILAFLEAKLQELDGPGARLADYFDCIAGTSTGGLITAMITAPGKDGRPLFAAKDVNRFYLDNGPYIFPQRCALAAVTASLRRPRYNGKYLHGKIRSMLGETRLCDALTDVVIPTFDVKLLQPIIFSTYDAKSMPLKNARLADVCIGTSAAPTYLPAHHFHTHDGNGKEREYNLIDGGVAANNPTMVAMTQITKKMMGKDREELYPVEPSDCGKFLVLSVGTGSTSDQGLYTAKQCSQWGIISWLRNKGMAPIIDIFMAASSDLVDIHAAVLFQSLHSDANYLRIQDNSLHGPAATVDAATPENMAELLRIGERMLAQRVSRVNVETGRYEEVKGAGSNADALAGFARQLSDERRTRLGRRRGGAGRLKSSR

SEQ ID NO: 84 - DNA, 200 bp, Triticum aestivum
gtggacgctgccacgcccgagaacatggcggagctcctcaggatcggcgagcggatgctggcgcagagggtgtccagggtgaacgtcgagaccgggaggtacgaggaggtaaagggggccgggagcaacgccgacgcgctcgccgggttcgccaggcagctctccgacgagaggaggacaaggctcgagcgccggcgtgg

SEQ ID NO: 85 - DNA, 200 bp, Triticum aestivum
gtggacgccgccacgcccgagaacatggcggagctcctcaggatcggcgagcggatgctggcgcagagggtgtccagggtgaacgtggagaccgggaggtacgaggagataaagggggccgggagcaacgccgacgcgctcgccggcttcgccaggcagctctccgacgagaggaggacaaggctcggccgccggcgcgg

SEQ ID NO: 86 - DNA, 200 bp, Triticum aestivum
gtggacgccgccacgcccgagaacatggcggagctcctcaggatcggcgagcggatgctggcgcagagggtgtccagggtgaacgtcgagaccgggaggtacgaggaggtaaagggggccgggaacaacgccgacgcgctcgccggcttcgccaggcagctctccgacgagaggaggacaaggctcgggagccggcgcgg

SEQ ID NO: 87 - DNA, 50 bp, Artificial Sequence
  Consensus sequence
acgaggaggtaaagggggccgggagcaacgccgacgcgctcgccggcttc

SEQ ID NO: 88 - DNA, 23 bp, Artificial Sequence
  target TTTN_AS+DL
cgagaccgggaggtacgaggagg

SEQ ID NO: 89 - DNA, 23 bp, Artificial Sequence
  target TTTN_BL
ggagaccgggaggtacgaggaga

SEQ ID NO: 90 - DNA, 567 bp, Triticum aestivum
acgatggtggcgatgacgcagatcaccaagaagatgatgggcaaggacagggaggagctgtacccggtgaagccgtcggactgcggcaagttcctggtgctgtccgtcggcaccggctcgacgtccgaccaggggctgtacacggcgaagcagtgctcccagtggggcatcatcagctggctccgcaacaagggcatggcgcccatcatcgacatcttcatggccgccagctccgacctcgtcgacatccacgccgccgtgctcttccagtcgctgcacagcgacggcgactacctccgcatccaggacaactcgctccacggcccggcggccacggtggacgctgccacgcccgagaacatggcggagctcctcaggatcggcgagcggatgctggcgcagagggtgtccagggtgaacgtcgagaccgggaggtacgaggaggtaaagggggccgggagcaacgccgacgcgctcgccgggttcgccaggcagctctccgacgagaggaggacaaggctcgagcgccggcgtggtggcgccggccgtctgaaatccagacgctga

SEQ ID NO: 91 - DNA, 567 bp, Triticum aestivum; identical to SEQ ID NO: 13

SEQ ID NO: 92 - DNA, 567 bp, Triticum aestivum; identical to SEQ ID NO: 12

SEQ ID NO: 93 - DNA, 567 bp, Triticum aestivum; identical to SEQ ID NO: 11

SEQ ID NO: 94 - DNA, 567 bp, Triticum aestivum; identical to SEQ ID NO: 90

SEQ ID NO: 95 - DNA, 568 bp, Triticum aestivum
acgatggtggcgatgacgcagatcaccaagaagatgatgggcaaggacagggaggagctgtacccggtgaagccgtcggactgcggcaagttcctggtgatgtccgtcggcaccggctcgacgtccgaccaggggctgtacacggcgaagcagtgctcccagtggggcatcatcagctggctccgcaacaagggcatggcgcccatcatcgacatcttcatggcagccagctccgacctcgtcgacatccacgccgccgtgctcttccagtcgctgcacagcgacggcgactacctccgcatccaggacaactcgctccacggcccggcggccacggtggacgctgccacgcccgagaacatggcggagctcctcaggatcggcgagcggatgctggcgcagagggtgtccagggtgaacgtcgagaccgggaggtacgaggaggtaaagggggccgggagcaacgccgacgcgctcgccgggttcgccaggcagctctccgacgagaggaggacaaggctcgagcgccgggcgtggtggcgccggccgtctgaaatccagacgctga

SEQ ID NO: 96 - DNA, 567 bp, Triticum aestivum; identical to SEQ ID NO: 11

SEQ ID NO: 97 - DNA, 567 bp, Triticum aestivum; identical to SEQ ID NO: 11

SEQ ID NO: 98 - DNA, 567 bp, Triticum aestivum
acgatggtggcgatgacgcagatcaccaagaagatgatgggcaaggacagggaggagctgtacccggtgaagccgtcggactgcggcaagttcctggtgctgtccgtcggcaccggctcgacgtccgaccaggggctgtacacggcgaagcagtgctcccagtggggcatcatcagctggctccgcaacaagggcatggcgcccatcatcgacatcttcatggccgccagctccgacctcgtcgacatccacgccgccgtgctcttccagtcgctgcacagcgacggcgactacctccgcatccaggacaactcgctccacggcccggcggccacggtggacgccgccacgcccgagaacatggcggagctcctcaggatcggcgagcggatgctggcgcagagggtgtccagggtgaacgtcgagaccgggaggtacgaggaggtaaagggggccgggagcaacgccgacgcgctcgccggtttcgccaggcagctctccgacgagaggaggacgaggctcgggcgccggcgcggtggcgccggccgtctgaaatccagccactga

SEQ ID NO: 99 - DNA, 567 bp, Triticum aestivum; identical to SEQ ID NO: 13

SEQ ID NO: 100 - DNA, 567 bp, Triticum aestivum; identical to SEQ ID NO: 13

SEQ ID NO: 101 - DNA, 567 bp, Triticum aestivum; identical to SEQ ID NO: 13

SEQ ID NO: 102 - DNA, 567 bp, Triticum aestivum; identical to SEQ ID NO: 13

SEQ ID NO: 103 - DNA, 567 bp, Triticum aestivum; identical to SEQ ID NO: 13

SEQ ID NO: 104 - DNA, 567 bp, Triticum aestivum; identical to SEQ ID NO: 13

SEQ ID NO: 105 - DNA, 567 bp, Triticum aestivum
acgatggtggcgatgacgcagatcaccaagaagatgatgggcaaggacagggaggagctgtacccggtggagccgtcggactgcggcaagttcctggtgctgtccgtcgggaccggctcgacgtccgaccaggggctgtacacggcgaagcagtgctcccagtggggcatcatcagctggctgcgcaacaagggcatggcgcccatcatcgacatcttcatggccgccagctccgacctcgtcgacatccacgccgccgtgctcttccagtcgctgcacagcgacgccaactacctccgcatccaggacaactcgctccacggcccggcggccacggtggacgccgccacgcccgagaacatggcggagctcctcaggatcggcgagcggatgctggcgcagagggtgtccagggtgaacgtcgagaccgggaggtacgaggaggtaaagggggccgggaacaacgccgacgcgctcgccggcttcgccaggcagctctccgacgagaggaggacaaggctcgggagccggcgcggtggcgccggccgcctgaaatccagccgctga

SEQ ID NO: 106 - DNA, 567 bp, Triticum aestivum; identical to SEQ ID NO: 105

SEQ ID NO: 107 - DNA, 567 bp, Triticum aestivum; identical to SEQ ID NO: 105

SEQ ID NO: 108 - DNA, 567 bp, Triticum aestivum; identical to SEQ ID NO: 12

SEQ ID NO: 109 - DNA, 567 bp, Triticum aestivum; identical to SEQ ID NO: 12

SEQ ID NO: 110 - DNA, 567 bp, Triticum aestivum; identical to SEQ ID NO: 12

SEQ ID NO: 111 - protein, 426 aa, Zea mays
MAGSLAASAFFPGPGASPAASAKNLAGEVPDSLSVRGIVAKPNANSGNMQVKAQAQTLPKVNGTKVNLKNASSDTEEAIPYTAPKTFYNQLPDWSMLLAAVTTIFLAAEKQWTLLDWKPKKPDMLVDTFGFGGIIQDGMVFRQNFIIRSYEIGADRTASIETLMNHLQETALNHVKTAGLLGDGFGATPEMSKRNLIWVVSKIQLLVEKYPLWEDTVQVDTWVAAAGKNGMRRDWHVLDCKSGCTILRATSVWVMMNKNTRRLSKMPDEVRAEIGPYFNARAAITDEQSEKLAKPGSTAGGDAMKQFMRKGLTPRWWGDLDVNQHVNNVKYIGWILESAPIAILEKHELASMTLDYRKECGRDSVLQSLTTVAGECVDGDTDSTIQCDHLLQLETGADIVKAHTEWRPKRAHGEGTPMGGFPAESA

SEQ ID NO: 112 - protein, 434 aa, Zea mays
MAGSLAASAFFPGPGASPAASAKTSKNLAGELPDNLSVRGIVATPNAPSGNMQVKAQAQALPKVNGTKVNLKNASPDTEEAIPYIAPKTFYNQLPDWSMLLAAVTTIFLAAEKQLTLLDWKPKKPDMLVDTFGFGRIIQDGLVFRQNFWIRSYEIGADRTASIETLMNHLQETALNHVKTAGLLGDGFGATPEMSKRNLIWVVSKIQLLVEQYPSWGDTVQVDTWVAAAGKNGMRRDWHVRDENSGRTILRATSVWVMMNKNTRRLSKMPDEVRAEIGPYFNGRSAITDEQSEKLAKPGSAPDGGTMKQQFIRKGLTPRWGDLDVNQHVNNVKYIGWILESAPISILEQHELASMTLDYRKECGRDSVLQSLTTVAGERMDGHAGDSTIQCQHLLQLESGADIVKAHTEWRPKRARGEGSLRGASGVFRPRMHE

SEQ ID NO: 113 - protein, 427 aa, Triticum aestivum
MAGSVAASGFFPTPGSSPAALARGSKNMSGEVPETLSVRGMVAKPNTPPASMQVKARAQALPKVNGSKVNLKTTGSDKEDTVPYTSSKTFYNQLPDWSMLLAAVTTIFLAAEKQWTMLDWKPKRPDMLVDTFGFGRIIQDGLVFRQNFLIRSYEIGADRTASIETLMNHLQETALNHVKTAGLLGDGFGATPEMSKRNLIWVVSKIQLLVEHYPSWEDMVQVDTWVASAGKNGMRRDWHIRDYNSGRTILKATSVWVMMNKTTRRLSKMPDEVRGEIGPHFNDRSAITEEQGEKLAKPRNKVVDPANKQFIRKGLTPKWGDLDVNQHVNNVKYIGWILESAPISILEKHELASMTLDYRKECCRDSVLQSLTNVSGECVDGSPDSAIQCDHLLQLESGADVVKAHTTWRPKRAHGEGNLGLFPVESA

SEQ ID NO: 114 - DNA, 6400 bp, Triticum aestivum
  misc_feature (1381)..(1698): n is a, c, g, or t
acttcagttacacccccaaaatcatgcatctcaggcttgaggaaaatgatgacatcgtctacgtaaatagatgcacggtgtttaacaagttggttacctagaggggttaggatgttgttcctttgtgctttgtccatcaaccttccaaggcagtccaccacaagcacaaagagcagcggggagaggggatctccttgcccgaggcccttacgatggaggatttgttgagtgcatttggagttgactaaaattcttgtggaagcagagaaaaagagggctcatatccatcctctccacttcggcccaaagccctgaccttgcagtagcttaagcagaaactcccatgaaactgaatcaaaggctttggatatatccaacttaagcataacagaggggattttttttctgccttagggtcttagccattgcctgaacatagacaaaattatcatgtatgcttcgccctttaatgaatgcattctgacatggggaggttagcttgatcatccatggttgtagtctggcagccatcatcttggcaattaattttgcgaagaagtggattaggctaatgggcctaaaatcttgaggcctaacagcaccctggtgctttggaagcaaggcaataatagcactattaagcagatttttaataactatatatatactagtgtacgtacaattatttgatttgttgtggcctgctctaaatcatatagtatatataggtacataattttcgcagcgtcgcgatttgcgacggcctcttctcggtcccgtgagctgtcgaggtagtcccgggagaaggcttgctttctcaacccaccgtttccgttccgttcgcgtgctgattggagactcgtgcggtgcaggaagcatcggacagctacgagaaggagaaaaagggcgcggcccggggccccaccgctgcgggtcggaccgcaccgaggggcacgcgcgtcttttcgcggcgccagcgcagggcatacgggccccgtgccagccgtacgcggggccgcgtatttatacggcggcccgggacgggacgggggcgctaacggctctgcttcctcccctcccctcccgtcgtggccgccaacaaagagagaagagaagagagagaggaaaaaactcatctcgcgctcgctatcccttctcttttcttttctctctccttctcgtggtgtgtgaggtggcgaggtagaggaagcagcggagaccacacgaatccccccaccaccctcgcgcccccctcccccgtcccctctccctgcgcggcgccaccaacctcgcctctcccggcgcggccagaggagcccagcggagccgccggcgggtctgggtcaggtccggcctcgtccctcccgcggaggcggcagcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctccttctccttcccctgcggcgatctggcggcggtccgattcgttcgttcgttcgtcgcggggtttccgggctccgcattttggccccgcgcggcagatctggaggactgcatcctcgtcatccccttgcggtgggcgaattttttttcccgatgcgacaacgcggttttggcccgttccgcctcggttcctgcgatagggttcgtgcgggcagcgttggattcggggggcaattcggtactgttttcagatttgttttgtgtgggacgcacggatggatacggctcgagcggccgggacggttcccgtcgcttcaaggagaagggggcatttattttgcgactattatgttatggtaaattcggttgcaaacacggccccaatagtgaaggatgtttggatgtcaacaacgactccggggtctggtcgttggaggtttgggaaaattgcttcgagtggggcaaatttaaggctccctatctttttccccaaacgaggggcagcgggatcgggtggctcatctcaattttacatggaatcttttgttattcgtgaatgttgttgttctgtcacattttattccgtttcccttctcttctcttccacgagggaaattaggcgtagctattatgggtatgtataaatcctgttgctcatccttttgttcgttcttccttcaggttggagcgcaatcttggttcctgaagctgtagtacggctgtgtcatggcggggtctgttgccgcctcggggttcttccccacgccggggtctaccccggctgcgttggctagaggctccaagaacatgtctggtgaattacctgagactttgagtgtccgtgggatggttgcaaagccaaacacgccgcccgcgtccatgcaagtcaaggctcgggcccaagcacttcccaaggttaatggctctaaggttaacctcaagactacaggctcagacaaggaggatacagtgccttacacttcctcaaagaccttctataaccaactgcccgactggagcatgcttcttgcagctgtcacgaccatcttcctggccgcggagaagcagtggacgatgcttgattggaagcccaagagacctgacatgcttgttgacacatttggctttggtagaataatccaggatgggctggtgtttaggcagaacttcttgattaggtcctacgagattggtgcagatcgtacagcttctatagagacgttaatgaatcatttacaggtgagacaatgacaggaaacagctttcattttttattacttccatttttttcttgtttcattattgatgccatcagttcacaccagtcttctaaaacaattcgatttttggcaggaaacagctcttaatcatgtgaaaactgctggtctccttggagatggttttggcgctacgccagagatgagtaaacggaacttgatctgggttgtcagcaaaattcagcttcttgtagagcattatccctcatggtactttctgcaagccataattgttgatgtcattttcctggcaaatcccctctctttttctcactttccgacttccagtcaaattcatagcttgaattattcatgtgcattctttatggatgtgcaccctttcactggagtagtgttttttttttgtcatgttttttggtacacattttgaagtcatacttgtatattgcatcttattttatcttaatctcagaatgtagttttaagtagctaaactggagttagtcttgtttctttcctatttagcatcataacattgtacaaattgccttttgagtttgtcaatatgaacttccattggttgtcatattaaccattaaaagttccttatcacaactaagttggccttacgtcatactatgagaaatctatagttaagtacacgtaatatatttggagagattatgcaccactcaaacatgctctaagcccacttttttagtacaaacaatacatcatgtcctagttttgctcaactttgtggtcttatctaccacatcaattgctaaaacagtgacgatttatggaatggcaatacaagttaaggactatttatgtatgccatgggaagaatatgaaaaataatgtcagcagtgcaggcttagttcctgctagaatttataatgattgtttaccaatcagaagctcatgtgcactgtttatcatcctacatgaaaaactcccccatctatgtttctgattatgttgttctacacagatgtgttagattgttgcttcgcttttgctagaatttagaatgcttcttttagcaatcagaagttctgctcacagaatttgtttcacgcgtggaacaccttaaatcgattgttccacttgagtgtaacagacgtagattttgttttttgatgaataaacgatcatctttttcttgtgtctatgtatgcaaggtagaggtatctagtttgattgataagaccacgtattaaatgctcgaagctactaaattaagtggtgctggcccatttgtcctttgttattcgtattaaaagaggtcgggcctcaggagatcagaaagttgcctttattgctggattaaatgacttcttcgtatgcaattagtaccgtgggctgccaaaagaagaaactcctgatctagattggcatagtttttcttaaatacttctattagcctgtgtacctgatactacagttttcctttgatgtcagcaacggtcatttgttgaattgataaaaggttgaatcttaagtttgcagaaaaggaaaagcaatttcagtttgtagttttctagttgttctgtagctttactcgctgatttataggctgaaaaatgacctagtataattttcatgtgctaaattttagggaagatatggtccaagttgacacatgggtagcttctgctggaaaaaatggaatgcgtcgagattggcatatccgcgactacaattcgggcagaacaatcttgaaagctacaaggtttggattacatgtgtttgcgttttgattcatgctgtttgtagcatctttttcgtctgttaattgtgcttcccatttgacacttgtttccagtgtttgggttatgatgaataagaccactagaagactttcaaaaatgccagatgaagttagaggtgaaataggcccgcactttaacgaccgttcagccataacagaggagcagggtgaaaagttggctaagccaaggaacaaagttgtggaccctgccaacaagcagttcataaggaaggggcttactgtaagtcagttacatattcttaattcccatcactcagtttggtgtctctgtcatccataatatttctatttcttatgggaactccttcatcaacagcccaagtggggtgaccttgacgtgaaccaacatgtgaacaatgtgaagtatattgggtggatccttgaggtaatttattttccctattctcttgtgtttggtcaaaatggtctggctacttttctgcctaatacatgaactctggtggtaccaccagaagtttcatgcatctattattgtggagcatcaggtctggtgtttcataaagtagacatgattattctcattcccaatcatcatcagactgcttaagattcctgagctttatttattcccatttcagagtgctcctatttcaatactggagaagcatgagcttgcaagcatgacactggattacaggaaggagtgttgccgtgacagtgtgctgcagtcacttaccaacgtttcaggtgaatgtgcggatggcagcccagactctgccatccagtgcgaccatctgctccagctggagtctggagctgatgttgttaaggctcatacaacttggcggccaaagcgaccgcatggcgaaggaaaccttgggctgttcccggttgagagtgcataaccgcttcgaatgttaaatacggcaagcatgggttcttatgggatgcaaattctaatcttgccggggataaatggttgattgaaggaattattttgtgccggggataagccgcatagcccagcctgctgcatcttatgctacacacacattgtgatggagagatagagcttgttcaaactgttttcggaggaattggtcggatttgacaggctttggaggaatggggagcttgactggtgatactgaagtaatttggaggacagagaagggtggaatttggattatgggacaaggcaggggcttgtgtgtggtgatagtgatttgagctgattggtgaagaaggaagaatgcagcggcgttggctgggattgtttgtgtttttgacatgtgtttggacgcggagcggaatggataaataatttgtgtactaagttagcccagatgtgtgtgtggaggcgaaatcctatcccttgtaacagcctgatgatttgtacattcctacctagtacccccctcttctcttctcttctcttgttggttgaatcgaatcgaatcgcagctctcctgcgtttccttccttggaaacacatgcgttactggtgtcatgtgataaccccttatgcttgatagttctctcttccagctgattttagtctgcgcttttactgtgctccttatcatgtgccagatgaccttactctgtcacattgaccctagctgttttgtggtcgttgctgcgaggagacgggccctttcttgtggggctagtgttggagttgtctgacgcgtgcagctggcgc

SEQ ID NO: 115 - protein, 427 aa, Triticum aestivum
MAGSVAASGFFPTPGSTPAALARGSKNMSGELPETLSVRGMVAKPNTPPASMQVKARAQALPKVNGSKVNLKTTGSDKEDTVPYTSSKTFYNQLPDWSMLLAAVTTIFLAAEKQWTMLDWKPKRPDMLVDTFGFGRIIQDGLVFRQNFLIRSYEIGADRTASIETLMNHLQETALNHVKTAGLLGDGFGATPEMSKRNLIWVVSKIQLLVEHYPSWEDMVQVDTWVASAGKNGMRRDWHIRDYNSGRTILKATSVWVMMNKTTRRLSKMPDEVRGEIGPHFNDRSAITEEQGEKLAKPRNKVVDPANKQFIRKGLTPKWGDLDVNQHVNNVKYIGWILESAPISILEKHELASMTLDYRKECCRDSVLQSLTNVSGECADGSPDSAIQCDHLLQLESGADVVKAHTTWRPKRPHGEGNLGLFPVESA

SEQ ID NO: 116 - DNA, 6480 bp, Triticum aestivum
  misc_feature (5973)..(6223): n is a, c, g, or t
gttggcagagggtagccgatggaaggccagacttgcccggccgagatcgttttattttcttccctaggaagaaagaaaacgcgtgagccccagagcgtaactaatcttctttatttatgaatcattgagaaaagaagaaggcgtattcttttggcggattacaatccatcaataattatgacactaaaggcgagagcagacaaataaattacaagcttgcgtaaatgcccatataggcttgatttctgttggcattttagacaacttacgcgagcgtgataatagaaaagaccacatctgagtgggattggcaaaaagacgacctgccatgtcccatggcggcaagtgcgacacctggcactagcaggtggtctaaaatgccaaaattctctagatttagggctcgtgtaaaatggaatgtggggaaacaaaaccaaagcacgcgagatgctcatagcgagatcaagtcaccggactcccttcccatgtgtctccgccgctgtatccgtcggagtttgaaaggggaggaaggccgagtgcagagagaacgaaaaataaccgagaaattaaagcataccaacatggatcttactcgccgaggttttttataacaaagctacaaatctccgttcattagatttgtgtcaagtcaaattctatgaagtttgcaaattaaaatggtcaagaggaagtactctctaaaattgtaccaaagctgtgtatgtaatttttcattatttttggacatgattgaagatgaatgtatcgacaggttttcgaaaagaaaataataataatattgatccgaaggaagggcgtatttggcagcgtagcgatttgcaacggccacgtcgcggtgccgtgagctgtcgagtcccgggagaagagccttgctttctcaacccaccgtctccgttggcgtgctgattggagactcgtgcagtgcaggaaccatcggacagctacgagaaggaggaaaagggcgcggcgcgggccccacggcagcccggaaccagccacgctgggggccggtcggaccgcaccgagggcgaggggcacgcgcgtcttttcgcgcacgcgtatacgggcccccgtgggccgtacgcggggcgcgtatttatacggcggccccgacccgtgagcgctaacggctctgcttcctcctcccctcccgtcgtggccgcctcgcaaacagagagagaaagagagaaagagaggaaaaaaaactcatctgccccccgactctctctctcgctctatccctttcctcttctctctccttctcgtggtgtgtgaggtggcgaggtagaggaagcagcggagaccacacgaatccccccaccgccctcgcgccccccctcccctcccagcgcggcaccaccaccaacctcgcctctcccgtcgcggcgcggcgagaggagcggagccgagcgccgccggagccggcggcgggtctgggtcaggtccggcctcgtccgtcccgcggaggcggcggcagcgcgcgcgcgcgcggtgagtccctctccctccccctcctccccccgcggcgatctggcggcgggtcgattcgctcgggattccgtcgcgggctttccgggctctgcattttggccccgcgcggcagatctggcgggccgcgtcgctctccgcccccttgcggtggggatttttttctctctcgatgcgacaacgcggtttggcccgtttcgccccggttcctgcgatagggttcgcgcgggcagcgttggattcggtggcaatgcggtgctgttttcagatttgttttgtgtgggacggacggatggacagggcccgagcggccgggacggttcccgtcgcgctccaaggagaagggggccatttattttgcgattattatgtcatggtaaattcggctgcaaacacggccccaatagcaaaggatgtttggatgtcaacaacgaccctgaccgtctccggggcctggtcgttggaggtttggggaaattgattcgaatggggcaaatttaaggctccctatccttttccccgaatgaggaacagcgggatccagtggctcatatctcaattttacatggaatctttttgttgttccttgttcgtgaatgttgttgttctgtcagattttatttcgtctcccttctcttctcctccacgagggaaattaggcgtagctattatgggtgtgcataaatcctgttgctcatccttttgttccttctccttcttctttcaggttggagcgtaatcttggtttcctgaagctgtagtacggctgtgtcatggcggggtctgttgccgcctcggggttcttccccacgccgggctcttccccggctgcgttggctagaggctccaagaacatgtctggtgaagtacctgagactttgagtgtccgtgggatggttgcaaagccaaacacgccgcccgcgtccatgcaagtcaaggctcgggcccaagcacttcccaaggttaacggctctaaggttaacctcaagactacaggctcagacaaggaggatacagtgccttacacttcctcaaagacgttctataaccaactgcccgactggagcatgcttcttgcagctgtcacgaccatcttcctggccgcagagaagcagtggacgatgcttgattggaagcccaagagacctgacatgcttgttgacacatttggctttggtagaatcatccaggatgggctggtgtttaggcagaacttcttgattaggtcctacgagattggtgcagatcgtacagcttctatagagacgttaatgaatcatttacaggtgagacaatgacaggatgcagcttccattttttttctcgtttcactattgatgccatcagttcacatcagtcttcttaaacaaattcaaattttggcaggaaacagctcttaatcatgtgaaaactgctggtctccttggagatggttttggcgctacgccggagatgagtaaacggaacttgatctgggttgtcagcaaaattcagcttcttgtagagcattatccctcatggtacttttctgcaagccataattgttgatgtcatttttctggcaaatcctctctccttttctcactttcagattttcagtcaaattcatagctcgaactactcatgtgcattctttatggatgtgcaccctttcactggagtagtgttttttttttgtcatgctttttggcacacattttgaagccatacctgtataatgcgtcttattttatcttaatctcagaatgtaatgttaattacctaaactggagtcagtcttttttctttcctatttagcatcataacattctacaaactgccttgtgagtttgtcgatatgaacttccattggttgtcatattaaccatgaaaaatttcttataactaagttggccttacgtcattactatgagaaatccatagttaagtacaggcaatatatttggagagattatgcaccactcaaacatgctctatgcccacttttttagtacaaacaacacatcatgtcctagtttcgctcaacgttgtggtcttcttatttaccacattgattgctaaacacagtggcgatttatggaacggcaatacaagttcatgactatttatgtatgccatgggaagaatatatgaaaaataatgtcagcagtgcaggcttggttcctgctagaattaacgattgtttacaaatcagaagcttatgtgtactgcttatcatcctacatgaaaaactcccctttctatgtttctgattctgttgttctacacagatgcgttagattgtggctccacttttgctagaatttagaatgcttctttagcaatcagaagttctgctcacagtcacagagtatgtttcacgtgtggaacaccttaaatcgattgttccacttgattgtaacagacatagattttgtttttttgatgaataaacgatcatcttttcttgtgtctacctctgcaaggtagaagaatctagtttgattgataagaccacgtattaaatgctcgaagctaccaaattaagtggtgctggcccatttgtcctttgttattcgtattaaaagaggtcgggcctcaggagatcagaaagttgcctttattgctggattaaatgacttgatcatatgcaattagtgctgtgggctgccaaatgaagaaactgatctagattggcataatttttcttaaatacttttattggtctgtgtacctgatactgcagttttcctttgatgtcagcaacgatcatttgttgaattgataagaggttgaatcttaagtttgcggaaaaggaaaagcaatttcagtttggtagtgttctagttgttctgtagctttacttggtgatttacaggctgaaaaatgacctagtataattttcatgtgctaaattttagggaagatatggtccaagttgacacatgggtagcttctgctggaaaaaatggaatgcgtcgagattggcatatccgtgactacaattcgggcagaacaatcttgaaagctacaaggtttgaattccatgtgtttgcattttgagtcatgctgtttgtagcatctttttcgtctgttaattgtgcttcccatttgacactttgtttgcagtgtttgggttatgatgaataagaccactagaagactttcaaaaatgccagatgaagttagaggtgaaatagggccgcactttaacgaccgttctgccataacagaggagcagggtgaaaagttggctaagccaaggaacaaagttgtggaccctgccaacaagcagttcataaggaaggggcttactgtaagtcagttagatattcttattaattaccatcactcagtttggtgtttgtgtcatctataatatttctttttcttatgggaactccttcatcctcaacagcccaagtggggtgaccttgacgtgaaccaacatgtgaacaatgtgaagtatattgggtggatccttgaggtaatttatttcccctatattctctagtgtttggccaaagtgttgtggctacttttctgccgaacacatgaactctgctggtaccaccagacgtcgtttcatgcatctattattgtggagcatcaggtctggtgtttgataaagtagacatgattgttctcattcccaatcatcatcatagtaagactgcttaagattcctgagctttatttattccctttccagagtgccccgatttcaatactggaaaagcatgagcttgcaagcatgacactggattacaggaaggagtgttgccgtgacagcgtgctgcagtcactgaccaacgtttcgggcgaatgcgtggatggcagcccagactctgccatccagtgcgaccatttgctccagctggagtctggagctgatgtggtcaaggctcacacaacttggcggccaaagcgagcgcatggcgaaggaaacctcgggctgttcccggtcgagagtgcgtaaccgcgtcaaatgttaaatacggcaagcatgggttgttatgagatgcaaattctaatcttgccggggataaatggttgattgaaggaattattttgtgccggagataagccaagccacacacattctgattcggggagtgcaacgaacatggtggcagcttgttcaaactgttttgggaggaaatggtagaatttgacagccttgggtggaacgaggagcttcactggtggtactgtagtaatttggtggatggggaatggtgaaatttcgagtatgggagaaggctggggcttgtgtgtggtgatagtggtttgagctgattggtgaaggaaggaaaggaaggctgcagcggcggcggctgggattgtttgtgttttttgacatgtgtttggatgcggagcgaaatggataaataatttgtgtactaagttagcccagatgtgtgtgtgtgtggaggcggaatcctatcccttgtaacagcctgatgatttgtacattcctacctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntctcctgtcttgttggttgaatcgaatcgcagcgcagctctcctgcgtttccttggttggaaacctatgcgtccgttgccgctgtcatgtgctagcccccatgcttgaaatgattctactgtatgctgtgccctcgttactgctcggaggggacgggctctttttttagagggagagaagacaggctctctcttgtggggtttgtgttttgagttgtctaagtgtgcagctggcgctgtgaatttccggaagatttg

SEQ ID NO: 117 - protein, 425 aa, Triticum aestivum
MAGSVASGFFPTPGSSPAASARGSKNMSGELPESLSVRGMVAKPNTPPASMQVKARAQALPKVNGSKVNLKTTGSDKEDTVPYTSSKTFYNQLPDWSMLLAAVTTIFLAAEKQWTMLDWKPKRPDMLVDTFGFGRIIQDGLVFRQNFLIRSYEIGADRTASIETLMNHLQETALNHVKTAGLLGDGFGATPEMSKRNLIWVVSKIQLLVEHYPSWEDMVQVDTWVASAGKNGMRRDWHIRDYNSGRTILKATSVWVMMNKTTRRLSKMPDEVRGEIGPHFNDRSAITEEQGEKLAKPRNKVVDPANKQFIRKGLTPKWGDLDVNQHVNNVKYIGWILESAPISILEKHELASMTLDYRKECCRDSVLQSLTNVSGECVDGSPDSAIQCDHLLQLESGADVVKAHTTWRPKRAHGEGNLGLFPVES

SEQ ID NO: 118 - DNA, 7400 bp, Triticum aestivum
tcgaggggcaccaaatcgtcttgtgcctagtgatgaaatgtctgaaatactcaatgcacacgattagtccacaaaagcattgtcatcaatcaccaaaacaactaagggataaatatgcccttacactcggtccaccagggttcaagccctggcgcttgcattgttcctggatttatttcaggatttctggcgatgcttgttcgaaataatatttaggccgagctcggctttcatttctcaccattttcggggcaggcttaaacgcgagtagactgaaaaactgcatttcaggccgggcttttgggcttcatgtcaggcttacacgtgagtagactgaaaaactgcatttcgggctgggctttcgagcttcatgtcaggcttacacgtgagtagactgaaaaactgcatttgggccgggcttttgggcttcgtttcaggcttacacgtgagtagtctgaaaaactgcatttgggtcaggcttgcatttgggctgggcttttgggcttgagaaagcacatactttcgggcttgggcttaggatttgcacttcggtctttttcaagcccggtccagggtatgatcaagtttactccaaagtagcttaccaaagactgatctagcaccccctgcttgactaaaacttcggaggagggaaccacacccagtagccacgaaacacacaaatctagcacatggtccaccatcttccagatgtcgtcgatgcataccacaatctgcatccactcctggactaccttccaagcttcgtgacggcactgaagcaaacaccatcgcaacgacggagcccgaggacacaagtccaccacaaggatgtcgtcgtccccacgccatcctcacttgaacagactagtttccaaatccatcctcgcccatagaattgatcacctcgtcgggaaaggatctggagcttctttatttatggtcgccatcaccgtcatcgaagtccatatgtcgaatgacctaaagcataagctactagggcctaaaagctaaagctacacgattgacacgtgcggatccgacgacacccctcaccacctacgaccaagaagtcgacggcggagtgtagacgacacaaggctagactagcttggccgagatcgctttcttttcttcctcacgaagaaagaaaatgtgtttgccaccaggcgtccaatcgtctttatttatgaaccgtcgagaaaagaagaaggcatattctttttgcagatcatcaataaatacaacactaaaaggcgataaccgacaaataaattacaagcttgcgtaaatgcccatctaggcttgatttttgggtagtttcgaccgcctacgcaagtgtgatgtcagaaaggaccacatctgagtggaattggcaaaaaaaatgcctgctccgcggcgcgtgtaggtggtctaaattgccaaaattctctagatttagggctcgcgtaaacgacgtgtaaggaaacaaaactagatcgcaccaaatcatcataggtaatctagcaccgaaagcttgcagcattgctcatctactcaatcaagcgaagcgaccaagtggaccagcaagagagaaaatggaagagctcgacttgatctcggctatcggatgacgaacatgcaagtcacaagccgccggcttccctcccccttgtctcaccgcggtcgaagccgtcggagttggaagggggaggaagggcgagtgcagatagagcagagaaataaccagaggaattatagcaggacaacatcgatcttactcatcgagatttttgtaaaaaaatgcttacagtgatttgattcgcagtggccttctctaaactatttgcaattttcgcggcgtagcaattttcgtcacggtcgcgtgagctgtctgagtcccgggagaagccttgctttctcaacccaccgttttccattcgcgtgctgattggaggctcgtgcggtgcaggaaccatcggacagctacgagaaggagaaggagaaaaagggcgcggcccggggccccaccgctgccggtcggaccgcaccgaggggcacgcacgtcctttcgcagcgccagcgcaaggcatacgggcccgtgccgtgagccagccgtacgcggggccgcgtatttatacggcgacccgggacgggacgggagcgctaacggctctgcttcctccctcccctcccgtcgtggccgcctcgcatcgcaaacaaagggagagaggaaaaaactcatctgcccccgactctctctctctcgctcgctatcccttttcttttctctctccttctcgtggtgtgtgaggtggcgaggtagaggaagcagcggagaccacacgaatccccccaccaccctcgcgcccccctcccagcgcggcgccaccaacctcgcctctcccggcgcgccgagcggagccgagcgccgccggagccggcggcgggtctgcgtcaggtccggcctcgtccgtcccgcggaggcggcggcagcgcagcgcgcgctcgcggtgagtcccctccccctcctcctcctcctcgctcctcctccccgggcgatctggcggcgggcgccttcgtcggggactccgttacgggctccgcattttggccccgatggggctcccttggcagatctggcggaccgcatcctcgtcatccccttgcggtgggggaattttttttcccgatgcgacaacgcggttttggcccgttccgcctcggttcctgcgatagggttcgtgcgggctgcgttggatttgggggcaatgtactagtaatgttttctagatttgttttgggtggaacggacggacggacgggtgggcatggataggggtcgagcggcggacaagtccccgttctggggtcgattcccccgttgcgctccaaggagaaggggacatttgttttgcgattattatgttatggtaaattcggttacaaacatggtcccaatgacaaacgatgtttggatgtcaacaacgactctgaccgtctccggggtctggtcgttggaggtttggaaaattgattcgaatggggcaaatttaaggctccctatccttttccccaaatgaggaacagcgggatccggttcctcatatatctcaattttgcatggaatcttttgttatttgtgaatgttgttgttctgtcacattttatttcgtctcctttctgctctcctccacgagggaaatcaggcgtagctattgtgggtatgtataaatcctgttgctcatccttttgttcgttcttccttcaggttggagcttaatcttggttcctgaagctgtagtagggctctgttatggcggggtctgtcgcctcggggttcttccccacgccggggtcttccccggctgcatccgccagaggctccaagaacatgtctggtgaattacctgagtctttgagtgtccgtgggatggttgcaaagccgaacacgcctcccgcgtccatgcaagtcaaggctcgggcccaagcacttcctaaggtcaatggctctaaggttaacctcaagactacaggctcagacaaggaggatacagtgccttacacttcctcaaagacgttctataaccaactgccagactggagcatgcttcttgcagctgtcacgaccatcttcctggccgcagagaagcagtggaccatgcttgattggaagcccaagagacctgacatgcttgttgacacatttggctttggtagaataatccaggatgggctggtgtttaggcagaacttcttgattaggtcctacgagattggtgcagatcgtacagcttctatagagacgttaatgaatcatttacaggtgagacaatgacaggatgcagcttccattttttattcctttcattttttttctcgtttcactattgatgccatcagttcacaccagtcgtctaaaaccagttgaattttggcaggaaacagctcttaaccatgtgaaaactgctggtctccttggagatggttttggtgctactcctgagatgagtaaacggaacttgatctgggttgtcagcaaaattcagcttcttgtagagcattatccctcatggtactttctgcaagccataattgttgatgtcattttcctggcaaatcttctctctttttctcactttcagacttccagtcaaattcctagcttgaattattcatgtgcattctttatagatgtgcaccctttcaccggagtagttttttttcctcatgttttttggtacacattttgaagtcatacttgtatcttgcatcttattttagctaacctggagttagtcttatttctttcctatttagcatcataacattgtacaaaaattgccttttgagtttgtcaatatgaacttccgttggttgtcatattaaccattaaaagtttcttataactaagttggccttacgtcatactatgagaaatccatagttaagtacaggcaatatatttggagagattatgcaccactcaaacatgctctaagcccacttttttagtacaaacaacacatcatgtcctagttatgctcaactttgtggtcttatttaccacatcgattgctaaaacagtgacgatttatggaatggcaatacaagttaaggactattttatgtatgccatgaaaaataatgtcagcagtgcaggcttagttcctgctagaatttataatgattgttttatcaatcagaagctcatgtgtactgcttatcatcctacatgaaaaactcccccctctatgtttctgattctgttgtcctacacagatgtgttagattgttgcttcgcttttgctagaatttagaatgcttctttagcaatcagaagttctgctcacagagtatgtttcacgtgtggaacaccttagatcgatcgttccacttgagtgtaacagacgtagattttgctttttgatgaataaacgatcatctttttcttgtgtctccgtgtgcaaggtagaggaatctagtttgattgataagaccacgtatttaaatgctcgaagctactaaattaagtggtgctggcccatttgtccttcgttattcgtattaaaagaggccgggcctcaggagatcagaaagttgcctttattgctggattaaatgacttgttcgtatgcaattagtgctgtgggctgccaaacgacgaaactcctgatctagattggcataatttttcttaaatacttttattagtctgtgtacctgatactacagttttcctttgatgtcagcaacgatcatttgttgaattgataagaggttgaatcttaagtttgcggaaaaggaaaagcaatttcagtttgtagctttctagttgttctgtagctttacttgctgttttataggctgaaaaatgacctagtataattttcatgtgctaaattttagggaagatatggtccaagttgacacatgggtggcttctgctggaaaaaatggaatgcgtcgagattggcatatccgtgactacaattcgggcagaacaatcttgaaagctacaaggtttggattccatgtgtttgcattttgactcgtgctgtttgtagcatctttttcgtctgttaattgtgcttcccatttgacacttgtttgcagtgtttgggttatgatgaataagaccactagaagactttcaaaaatgccagatgaagttagaggtgaaataggcccgcactttaacgatcgttccgccataacagaggagcagggtgaaaagttggctaagccaaggaacaaagttgtggaccctgccaacaagcagttcataaggaaggggcttactgtaagtcagttagatattcttaattaccatcactcagtttggtgtctctgtgtcatccataatatttctatttcttatgggaactccttcatcctcaacagcccaagtggggcgaccttgacgtgaaccaacatgtgaacaatgtgaagtatattggatggatccttgaggtaatttattttccccattctctagtgtttggtcaaaatgttgtggctatccgaatacatgaactctgctggtaccaccagacgattcatgcatctattattgtggagcatcaggtctggtgtttcataaagtagacatgattgttctcattcccaatcatcatcttaaggctgcttaagattcctgagctttatttattcccttttcagagtgctccgatttccatactggaaaagcatgagcttgcaagcatgacactggattacaggaaggagtgttgccgtgatagcgtgctgcagtcacttaccaacgtttcgggtgaatgcgtggatggcagcccagactctgccatccagtgcgaccatctgctccagctggagtctggagcggatgtggtcaaggctcatacaacgtggcggccaaagcgagcgcatggcgaaggaaaccttgggttgttcccggtcgagagtgcataaccgcttcagatgttaaatacggcaagcatgggttcttatgggatgcaaaatctaatcttgccggggataaatggttgattgaaggaattattttgtgccggggataagccaagccgcatagcccaacctgcatcttatatgccacacacacattctgattcggggactgcaacgaacatggtggcagcttgttcaaactgttttgggagaagatggtggaatttgagagccttgggtggaacgaggagcttcactggtggtacttcagtaatttggaggatggagaatggtggaatttcgactatgggacaaggcaggggcttgtgtgtggtgatagtgatttgagctgactggtgaagaaggaagaatgcaggcagcggcggctgggattgtttgtgttttttgacatgtgtttggatgccgagcgaaatggataaataatttgtgtactaagttagcccagatgtgtgtgtggaggcgaaatcctatcccttgtaacagcctgatgatttgtacattcctacctagtacccccctctcttctcttgtcttgttggttgaatcgaatcgcagcgcagctctcctgcgtttcctcccatgcttgaaaatggtccgtcgttgtttctgtcaaatgattttactctgggctttatttcttgaccctagttatgttgtgctcgctactccgaggagacgggctcgttttttttttttttgagggatagaagacgggctctttcttgtggggtgtcggttgaagttgtctcagagtgcgcagctggcgcagtgaatttccggaggattgtctcttgcctggctctggctggcgtttggtcgctgcgatgctgctttgattggtgaacaagaaggcacgaccacagcacagctccttgctgtgcgtgactgggaggtggagacgtgaagactgcgttcctagtctctttttcgggggaaaaaaaaactgaagctcc

SEQ ID NO: 119 - DNA, 513 bp, Triticum aestivum
atggcggggtctgttgccgcctcggggttcttccccacgccgggctcttccccggctgcgttggctagaggctccaagaacatgtctggtgaagtacctgagactttgagtgtccgtgggatggttgcaaagccaaacacgccgcccgcgtccatgcaagtcaaggctcgggcccaagcacttcccaaggttaacggctctaaggttaacctcaagactacaggctcagacaaggaggatacagtgccttacacttcctcaaagacgttctataaccaactgcccgactggagcatgcttcttgcagctgtcacgaccatcttcctggccgcagagaagcagtggacgatgcttgattggaagcccaagagacctgacatgcttgttgacacatttggctttggtagaatcatccaggatgggctggtgtttaggcagaacttcttgattaggtcctacgagattggtgcagatcgtacagcttctatagagacgttaatgaatcatttacag

SEQ ID NO: 120 - DNA, 512 bp, Triticum aestivum
atggcggggtctgttgccgcctcggggttcttcccacgccgggctcttccccggctgcgttggctagaggctccaagaacatgtctggtgaattacctgagactttgagtgtccgtgggatggttgcaaagccaaacacgccgcccgcgtccatgcaagtcaaggctcgggcccaagcacttcccaaggttaacggctctaaggttaacctcaagactacaggctcagacaaggaggatacagtgccttacacttcctcaaagacgttctataaccaactgcccgactggagcatgcttcttgcagctgtcacgaccatcttcctggccgcagagaagcagtggacgatgcttgattggaagcccaagagacctgacatgcttgttgacacatttggctttggtagaatcatccaggatgggctggtgtttaggcagaacttcttgattaggtcctacgagattggtgcagatcgtacagcttctatagagacgttaatgaatcatttacag

SEQ ID NO: 121 - DNA, 513 bp, Triticum aestivum; identical to SEQ ID NO: 119

SEQ ID NO: 122 - DNA, 512 bp, Triticum aestivum; identical to SEQ ID NO: 120

SEQ ID NO: 123 - DNA, 513 bp, Triticum aestivum
atggcggggtctgttgccgcctcggggttcttccccacgccgggctcttccccggctgcgttggctagaggctccaagaacatgtctggtgaattacctgagactttgagtgtccgtgggatggttgcaaagccaaacacgccgcccgcgtccatgcaagtcaaggctcgggcccaagcacttcccaaggttaacggctctaaggttaacctcaagactacaggctcagacaaggaggatacagtgccttacacttcctcaaagacgttctataaccaactgcccgactggagcatgcttcttgcagctgtcacgaccatcttcctggccgcagagaagcagtggacgatgcttgattggaagcccaagagacctgacatgcttgttgacacatttggctttggtagaatcatccaggatgggctggtgtttaggcagaacttcttgattaggtcctacgagattggtgcagatcgtacagcttctatagagacgttaatgaatcatttacag

SEQ ID NO: 124 - DNA, 512 bp, Triticum aestivum
atggcggggtctgttgccgcctcggggttcttcccacgccgggctcttccccggctgcgttggctagaggctccaagaacatgtctggtgaagtacctgagactttgagtgtccgtgggatggttgcaaagccaaacacgccgcccgcgtccatgcaagtcaaggctcgggcccaagcacttcccaaggttaacggctctaaggttaacctcaagactacaggctcagacaaggaggatacagtgccttacacttcctcaaagacgttctataaccaactgcccgactggagcatgcttcttgcagctgtcacgaccatcttcctggccgcagagaagcagtggacgatgcttgattggaagcccaagagacctgacatgcttgttgacacatttggctttggtagaatcatccaggatgggctggtgtttaggcagaacttcttgattaggtcctacgagattggtgcagatcgtacagcttctatagagacgttaatgaatcatttacag

SEQ ID NO: 125 - DNA, 513 bp, Triticum aestivum
atggcggggtctgttgccgcctcggggttcttccccacgccggggtctaccccggctgcgttggctagaggctccaagaacatgtctggtgaattacctgagactttgagtgtccgtgggatggttgcaaagccaaacacgccgcccgcgtccatgcaagtcaaggctcgggcccaagcacttcccaaggttaatggctctaaggttaacctcaagactacaggctcagacaaggaggatacagtgccttacacttcctcaaagaccttctataaccaactgcccgactggagcatgcttcttgcagctgtcacgaccatcttcctggccgcggagaagcagtggacgatgcttgattggaagcccaagagacctgacatgcttgttgacacatttggctttggtagaataatccaggatgggctggtgtttaggcagaacttcttgattaggtcctacgagattggtgcagatcgtacagcttctatagagacgttaatgaatcatttacag

SEQ ID NO: 126 - DNA, 513 bp, Triticum aestivum; identical to SEQ ID NO: 125

SEQ ID NO: 127 - DNA, 513 bp, Triticum aestivum; identical to SEQ ID NO: 125

SEQ ID NO: 128 - DNA, 513 bp, Triticum aestivum; identical to SEQ ID NO: 125

SEQ ID NO: 129 - DNA, 513 bp, Triticum aestivum; identical to SEQ ID NO: 125

SEQ ID NO: 130 - DNA, 513 bp, Triticum aestivum; identical to SEQ ID NO: 125

SEQ ID NO: 131 - DNA, 513 bp, Triticum aestivum; identical to SEQ ID NO: 125

SEQ ID NO: 132 - DNA, 513 bp, Triticum aestivum; identical to SEQ ID NO: 125

SEQ ID NO: 133 - DNA, 510 bp, Triticum aestivum
atggcggggtctgtcgcctcggggttcttccccacgccggggtcttccccggctgcatccgccagaggctccaagaacatgtctggtgaattacctgagtctttgagtgtccgtgggatggttgcaaagccgaacacgcctcccgcgtccatgcaagtcaaggctcgggcccaagcacttcctaaggtcaatggctctaaggttaacctcaagactacaggctcagacaaggaggatacagtgccttacacttcctcaaagacgttctataaccaactgccagactggagcatgcttcttgcagctgtcacgaccatcttcctggccgcagagaagcagtggaccatgcttgattggaagcccaagagacctgacatgcttgttgacacatttggctttggtagaataatccaggatgggctggtgtttaggcagaacttcttgattaggtcctacgagattggtgcagatcgtacagcttctatagagacgttaatgaatcatttacag

SEQ ID NO: 134 - DNA, 510 bp, Triticum aestivum; identical to SEQ ID NO: 133

SEQ ID NO: 135 - DNA, 510 bp, Triticum aestivum; identical to SEQ ID NO: 133

SEQ ID NO: 136 - DNA, 510 bp, Triticum aestivum; identical to SEQ ID NO: 133

SEQ ID NO: 137 - DNA, 510 bp, Triticum aestivum; identical to SEQ ID NO: 133

SEQ ID NO: 138 - DNA, 510 bp, Triticum aestivum; identical to SEQ ID NO: 133

SEQ ID NO: 139 - DNA, 510 bp, Triticum aestivum; identical to SEQ ID NO: 133

SEQ ID NO: 140 - DNA, 20 bp, Triticum aestivum
tgtctgagcctgtagtcttg

SEQ ID NO: 141 - DNA, 20 bp, Triticum aestivum
gcaagaagcatgctccagtc